SEQUENCE LISTING (13 sequences)

SEQ ID NO: 1 - protein, 1449 aa, synthetic construct
  REGION 1..1449: note = GTF 0768 mature protein with two N-terminal residues
VADQNVNDPSVATTTQNVVTDQDTSIDASVATTVNPNLDDTQADNTNIQTPTDQNDESKDTTPKVETGDTTNSQSTEAQETTAQTNNDVETPQNSDAAIETGLLTTNNQIRYVNPDGTVLTGAYKTINGNTYYFDDDSGVALVGLHKIGDTLKGFSLNGVQVKGDYLTAANGDKYYFDSNGNAVSGVQQINGKTYYFDSTGKLMKGYTAVLNGVVTFFNSTTGEADNTDASTIKTGVTIDNSDYTVHNAAYDNTAASFDNINGYLTAESWYRPKEILENGESWRPSTAEDKRPILITWQPDIVTEVNYLNMMAANGLLSINAPFTTASDLAIMNDAVRAVQKNIEMRISQEKSTDWLKALMTQFINTQPQWNEVSESPSNDHLQGGALTYVNSPLTPDANSNFRLLNRTPTNQSGTTRYDTDKSKGGFELLLANDVDNSNPVVQAEQLNWLYYLMNFGSITANDPTANFDGIRVDAVDNVDADLLQIASDYFKLAYGTSLSDTNANQHLSILEDWSANDAEYMSKTGSNQLTMDTYTQQQLLFSLTKQVGNRADMRRFLEYFMINRANDSTENVATPNYSFVRAHDSEVQTVIATIIKDLHPDVVNSLAPTQAQLEEAFAVYNADMNRVDKQYTQYNMPSAYAMLLTNKDTIPRVYYGDLYTDDGEYMGTQTPYYDAIVNLLQSRVKYVAGGQSMAVDQHDILTSVRYGKNLADANATSDDLTSINSGIGVIVSNNPNLSLASGETVVLHMGIAHANQVYREILETTDNGIANNTDIFKTTDSNGDLIFTASEIHGYSNVQVSGFLSVWAPKDATDDQDVRTAASESTSNDGNTLHSNAALDSNLIYEGFSNFQSTPQSESEFANVKIAANVNLFKSWGVTSFQMAPQYRSSTDTSFLDSIIQNGYAFTDRYDLGFETPTKYGTDQQLRDAIKALHANGIQAMADFVPDQIYNLPQTELVSVSRTDSLGNQSANSNAANVLYVSHTVGGGEYQSKYGGEFLAIIKSKYPSLFKTIQVSTGLPIDDSTKIKEWSAKYFNGSNIQGRGFGYVLSDGGTQNYFKVISNSTDDDFLPNQLTGKPTMTGFEQTSKGIVYYSKSGIQAKNQFVKDDVSGNYYYFNKNGLMTVGSKTINGKNYMFLPNGVELRGSFLQTADGTVNYYATNGAQVQDSYVTDTEGNSYYFDGDGEMVTGTYTVDGHAQYFDVNGVQTKGAIITLGGVQRYYQAGNGNLATNQYVSYNNSWYYANTKGELVTGVQSINGNVQYFASNGQQIKGQIVVTGNQKSYYDANTGNLIKNDFLTPDQGKTWYYADQDGNLVVGAQEVNGHKLYFDDNGIQIKDQIISNDGQQYYYQGGNGDLVTNRYISYNDSWYYADATGVLVTGQQIINGETQYFRTDGRQVKGQIIADGDKQHYYDADSGNLVKNNFVTVDQGKTWYYADQDGNLSLVDR

SEQ ID NO: 2 - protein, 1447 aa, Leuconostoc pseudomesenteroides
  REGION 1..1447: note = GTF 0768 mature wildtype protein
DQNVNDPSVATTTQNVVTDQDTSIDASVATTVNPNLDDTQADNTNIQTPTDQNDESKDTTPKVETGDTTNSQSTEAQETTAQTNNDVETPQNSDAAIETGLLTTNNQIRYVNPDGTVLTGAYKTINGNTYYFDDDSGVALVGLHKIGDTLKGFSLNGVQVKGDYLTAANGDKYYFDSNGNAVSGVQQINGKTYYFDSTGKLMKGYTAVLNGVVTFFNSTTGEADNTDASTIKTGVTIDNSDYTVHNAAYDNTAASFDNINGYLTAESWYRPKEILENGESWRPSTAEDKRPILITWQPDIVTEVNYLNMMAANGLLSINAPFTTASDLAIMNDAVRAVQKNIEMRISQEKSTDWLKALMTQFINTQPQWNEVSESPSNDHLQGGALTYVNSPLTPDANSNFRLLNRTPTNQSGTTRYDTDKSKGGFELLLANDVDNSNPVVQAEQLNWLYYLMNFGSITANDPTANFDGIRVDAVDNVDADLLQIASDYFKLAYGTSLSDTNANQHLSILEDWSANDAEYMSKTGSNQLTMDTYTQQQLLFSLTKQVGNRADMRRFLEYFMINRANDSTENVATPNYSFVRAHDSEVQTVIATIIKDLHPDVVNSLAPTQAQLEEAFAVYNADMNRVDKQYTQYNMPSAYAMLLTNKDTIPRVYYGDLYTDDGEYMGTQTPYYDAIVNLLQSRVKYVAGGQSMAVDQHDILTSVRYGKNLADANATSDDLTSINSGIGVIVSNNPNLSLASGETVVLHMGIAHANQVYREILETTDNGIANNTDIFKTTDSNGDLIFTASEIHGYSNVQVSGFLSVWAPKDATDDQDVRTAASESTSNDGNTLHSNAALDSNLIYEGFSNFQSTPQSESEFANVKIAANVNLFKSWGVTSFQMAPQYRSSTDTSFLDSIIQNGYAFTDRYDLGFETPTKYGTDQQLRDAIKALHANGIQAMADFVPDQIYNLPQTELVSVSRTDSLGNQSANSNAANVLYVSHTVGGGEYQSKYGGEFLAIIKSKYPSLFKTIQVSTGLPIDDSTKIKEWSAKYFNGSNIQGRGFGYVLSDGGTQNYFKVISNSTDDDFLPNQLTGKPTMTGFEQTSKGIVYYSKSGIQAKNQFVKDDVSGNYYYFNKNGLMTVGSKTINGKNYMFLPNGVELRGSFLQTADGTVNYYATNGAQVQDSYVTDTEGNSYYFDGDGEMVTGTYTVDGHAQYFDVNGVQTKGAIITLGGVQRYYQAGNGNLATNQYVSYNNSWYYANTKGELVTGVQSINGNVQYFASNGQQIKGQIVVTGNQKSYYDANTGNLIKNDFLTPDQGKTWYYADQDGNLVVGAQEVNGHKLYFDDNGIQIKDQIISNDGQQYYYQGGNGDLVTNRYISYNDSWYYADATGVLVTGQQIINGETQYFRTDGRQVKGQIIADGDKQHYYDADSGNLVKNNFVTVDQGKTWYYADQDGNLSLVDR

SEQ ID NO: 3 - protein, 1341 aa, synthetic construct
  REGION 1..1341: note = vGTFJ protein
VIDGKYYNANEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSYNDNHYNDKTDGAALAMENKQRLALLFSLAKPIKERTPAVSPLYNNTFNTTQRDEKTDWINKDGSKAYNEDGTVKLSTIGKYNEKYGDASGNYVYIRAHDNNVQDIIAEIIKAEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYSTNEVYTSVRYGKDIMTANDTEGSKYSRTSGQVTLVANNPKLTLDQSAKLNVEMGKIHANQKYRALIVGTADGIKNFTSDADAIAAGYVKETDSNGVLTFGANDIKGYETFDMSGFVAVWVPVGASDDQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANTKSSGKDYQAKYGGEFLAELKAKYPEMFKVNMISTGKPIDDSVKLKQWKAEYFNGTNVLERGVGYVLSDEATGKYFTVTKDGNFIPLQLTGNEKVVTGFSNDGKGITYFGTSGTQAKSAFVTFNGNTYYFDARGHMVTNGEYSPNGKDVYRFLPNGIMLSNAFYVDANGNTYLYNSKGQMYKGGYTKFDVTETDKDGKESKVVKFRYFTNEGVMAKGVTVIDGFTQYFGEDGFQAKDKLVTFKGKTYYFDAHTGNAIKDTWRNINGKWYHFDANGVAATGAQVINGQKLYFNEDGSQVKGGVVKNADGTYSKYKEGSGELVTNEFFTTDGNVWYYAGANGKTVTGAQVINGQHLYFNADGSQVKGGVVKNADGTYSKYDASTGERLTNEFFQTGDNNWYYIGANGKSVTGFVKIGDDTYFFAKDGKQVKGQTVSAGNGRISYYYGDSGKRAVSTWIEIQPGVYVYFDKNGIAYPPRVLN

SEQ ID NO: 4 - protein, 1341 aa, synthetic construct
  REGION 1..1341: note = Variant of GTF 6855 protein
VIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSYNDNHYNDKTDGAALAMENKQRLALLFSLAKPIKERTPAVSPLYNNTFNTTQRDEKTDWINKDGSKAYNEDGTVKQSTIGKYNEKYGDASGNYVYVRAHDNNVQDIIAEIIKAEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYSTNEVYTSVRYGKDIMTANDTEGSKYSRTSGQVTLVANNPKLTLDQSAKLNVEMGKIHANQKYRALIVGTADGIKNFTSDADAIAAGYVKETDSNGVLTFGANDIKGYETFDMSGFVAVWVPVGASDDQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANTKSSGKDYQAKYGGEFLAELKAKYPEMFKVNMISTGKPIDDSVKLKQWKAEYFNGTNVLERGVGYVLSDEATGKYFTVTKDGNFIPLQLTGNEKVVTGFSNDGKGITYFGTSGTQAKSAFVTFNGNTYYFDARGHMVTNGEYSPNGKDEYRFLPNGIMLSNAFYVDANGNTYLYNSKGQMYKGGYTKFDVTETDKDGKESKVVKFRYFTNEGVMAKGVTVIDGFTQYFGEDGFQAKDKLVTFKGKTYYFDAHTGNAIKDTWRNINGKWYHFDANGVAATGAQVINGQHLYFNADGSQVKGGVVKNADGTYSKYKEGSGELVTNEFFTTDGNVWYYAGANGKTVTGAQVINGQHLYFNADGSQVKGGVVKNADGTYSKYKEGSGELVTNEFFTTGDNNWYYIGANGKSVTGEVKIGDDTYFFAKDGKQVKGQTVSAGNGRISYYYGDSGKRAVSTWIEIQPGVYVYFDKNGIAYPPRVLN

SEQ ID NO: 5 - protein, 1341 aa, Streptococcus salivarius [sequence omitted]

SEQ ID NO: 6 - protein, 1341 aa, Streptococcus salivarius
  REGION 1..1341: note = GTF 7527. Position-1 methionine added.
MIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYRTNEVYTSVRYGKDIMTANDTEGSKYSRTSGQVTLVANNPKLNLDQSAKLNVEMGKIHANQKYRALIVGTADGIKNFTSDADAIAAGYVKETDSNGVLTFGANDIKGYETFDMSGFVAVWVPVGASDNQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANSKSSGKDYQAKYGGEFLAELKAKYPEMFKVNMISTGKPIDDSVKLKQWKAEYFNGTNVLERGVGYVLSDEATGKYFTVTKEGNFIPLQLTGKEKVITGFSSDGKGITYFGTSGTQAKSAFVTFNGNTYYFDARGHMVTNSEYSPNGKDVYRFLPNGIMLSNAFYIDANGNTYLYNSKGQMYKGGYTKFDVSETDKDGKESKVVKFRYFTNEGVMAKGVTVIDGFTQYFGEDGFQAKDKLVTFKGKTYYFDAHTGNGIKDTWRNINGKWYYFDANGVAATGAQVINGQKLYFNEDGSQVKGGVVKNADGTYSKYKEGFGELVTNEFFTTDGNVWYYAGANGKTVTGAQVINGQHLYFNADGSQVKGGVVKNADGTYSKYNASTGERLTNEFFTTGDNNWYYIGANGKSVTGEVKIGDDTYFFAKDGKQVKGQTVSAGNGRISYYYGDSGKRAVSTWIEIQPGVYVYFDKNGLAYPPRVLN

SEQ ID NO: 7 - protein, 1341 aa, Streptococcus salivarius
  REGION 1..1341: note = GTF 2678 protein. Position-1 methionine added.
MTDGKYYYVNEDGSHKENFAITVNGQLLYFGKDGALTSSSTHSFTPGTTNIVDGFSINNRAYDSSEASFELINGYLTADSWYRPVSIIKDGVTWQASTAEDFRPLLMAWWPNVDTQVNYLNYMSKVFNLEAKYTSTDKQADLNRAAKDIQVKIEQKIQAEKSTQWLRETISAFVKTQPQWNKETENYSKGGGEDHLQGGALLYVNDSRTPWANSNYRLLNRTATNQTGTINKSVLDEQSDPNHMGGFDFLLANDVDLSNPVVQAEQLNQIHYLMNWGSIVMGDKDANFDGIRVDAVDNVNADMLQLYTNYFREYYGVNKSEAQALAHISVLEAWSLNDNHYNDKTDGAALAMENKQRLALLFSLAKPIKDRTPAVSPLYNNTFNTTQRDFKTDWINKDGSTAYNEDGTAKQSTIGKYNEKYGDASGNYVFIRAHDNNVQDIIAEIIKKEINKKSDGFTISDSEMKQAFEIYNKDMLSSNKKYTLNNIPAAYAVMLQNMETITRVYYGDLYTDDGHYMETKSPYHDTIVNLMKNRIKYVSGGQAQRSYWLPTDGKMDNSDVELYRTSEVYTSVRYGKDIMTADDTEGSKYSRTSGQVTLVVNNPKLTLHESAKLNVEMGKIHANQKYRALIVGTADGIKNFTSDAEAIAAGYVKETDSNGVLTFGANDIKGYETFDMSGFVAVWVPVGASDDQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANSKSSGRDYQAQYGGEFLAELKAKYPKMFTENMISTGKPIDDSVKLKQWKAKYFNGTNVLDRGVGYVLSDEATGKYFTVTKEGNFIPLQLTGNEKAVTGFSNDGKGITYFGTSGNQAKSAFVTFNGNTYYFDARGHMVTNGEYSPNGKDVYRFLPNGIMLSNAFYVDANGNTYLYNYKGQMYKGGYTKFDVTETDKDGNESKVVKFRYFTNEGVMAKGLTVIDGSTQYFGEDGFQTKDKLATYKGKTYYFEAHTGNAIKNTWRNIDGKWYHFDENGVAATGAQVINGQKLYFNEDGSQVKGGVVKNADGTYSKYKEGSGELVTNEFFTTDGNVWYYAGADGKTVTGAQVINGQHLYFKEDGSQVKGGVVKNADGTYSKYDAATGERLTNEFFTTGDNNWYYIGSNGKTVTGEVKIGADTYYFAKDGKQVKGQTVTAGNGRISYYYGDSGKKAISTWIEIQPGIYVYFDKTGIAYPPRVLN

SEQ ID NO: 8 - protein, 1340 aa, Streptococcus salivarius
  REGION 1..1340: note = GTF 2919 protein. Position-1 methionine added.
MIDGKYYYVNKDGSHKENFAITVNGQLLYFGKDGALTSSSTYSFTQGTTNIVDGFSKNNRAYDSSEASFELIDGYLTADSWYRPVSIIKDGVTWQASTKEDFRPLLMAWWPNVDTQVNYLNYMSKVFNLDAKYTSTDKQVDLNRAAKDIQVKIEQKIQAEKSTQWLREAISAFVKTQPQWNKETENFSKGGGEDHLQGGALLYVNDPRTPWANSNYRLLNRTATNQTGTIDKSVLDEQSDPNHMGGFDFLLANDVDTSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKKAFEIYNKDMLSSDKKYTLNNIPAAYAVMLQNMETITRVYYGDLYTDDGHYMETKSPYYDTIVNLMKNRIKYVSGGQAQRSYWLPTDGKMDKSDVELYRTNEVYTSVRYGKDIMTADDTQGSKYSRTSGQVTLVVNNPKLSLDKSAKLDVEMGKIHANQKYRALIVGTPNGIKNFTSDAEAIAAGYVKETDGNGVLTFGANDIKGYETFDMSGFVAVWVPVGASDDQDIRVAASTAAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRNALKALHKAGIQAIADWVPDQIYQLPGKEVVTATRTDGAGRKISDAIIDHSLYVANSKSSGKDYQAKYGGEFLAELKAKYPEMFKVNMISTGKPIDDSVKLKQWKAEYFNGTNVLDRGVGYVLSDEATGKYFTVTKEGNFIPLQLKGNEKVITGFSSDGKGITYFGTSGNQAKSAFVTFNGNTYYFDARGHMVTNGEYSPNGKDVYRFLPNGIMLSNAFYVDGNGNTYLYNSKGQMYKGGYSKFDVTETKDGKESKVVKFRYFTNEGVMAKGVTVVDGFTQYFNEDGIQSKDELVTYNGKTYYFEAHTGNAIKNTWRNIKGKWYHFDANGVAATGAQVINGQHLYFNEDGSQVKGGVVKNADGTFSKYKDGSGDLVVNEFFTTGDNVWYYAGANGKTVTGAQVINGQHLFFKEDGSQVKGDFVKNSDGTYSKYDAASGERLTNEFFTTGDNHWYYIGANGKTVTGEVKIGDDTYFFAKDGKQLKGQIVTTRSGRISYYFGDSGKKAISTWVEIQPGVFVFFDKNGLAYPPENMN

SEQ ID NO: 9 - protein, 1340 aa, unidentified
  REGION 1..1340: note = GTF 2765 protein
  REGION 1..1340: note = GTF 2765 protein. Position-1 methionine added.
MIDGKYYYVNEDGSHKENFAITVNGQLLYFGKDGALTSSSTYSFTQGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSNYRLLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDVAALAMENKQRLALLFSLAKPIKERTPAVSPLYNNTFNTTQRDEKTDWINKDGSKAYNEDGTVKKSTIGKYNEKYGDASGNYVFIRAHDNNVQDIIAEIIKKEINEKSDGFTITDSEMKRAFEIYNKDMLSNDKKYTLNNIPAAYAVMLQNMETITRVYYGDLYTDDGNYMEAKSPYYDTIVNLMKSRIKYVSGGQAQRSYWLPTDGKMDKSDVELYRTNEVYTSVRYGKDIMTADDTQGSKYSRTSGQVTLVVNNPKLTLDQSAKLNVVMGKIHANQKYRALIVGTPNGIKNFTSDAEAIAAGYVKETDGNGVLTFGANDIKGYETFDMSGFVAVWVPVGASDDQDIRVAASTAAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRNALKALHKAGIQAIADWVPDQIYQLPGKEVVTATRTDGAGRKISDAIIDHSLYVANSKSSGKDYQAKYGGEFLAELKAKYPEMFKVNMISTGKPIDDSVKLKQWKAEYFNGTNVLDRGVGYVLSDEATGKYFTVTKEGNFIPLQLKGNKKVITGFSSDGKGITYFGTSGNQAKSAFVTFNGNTYYFDARGHMVTNGEYSPNGKDVYRFLPNGIMLSNAFYVDGNGNTYLYNSKGQMYKGGYSKFDVTETKDGKESKVVKFRYFTNEGVMAKGVTVVDGFTQYFNEDGIQSKDELVTYNGKTYYFEAHTGNAIKNTWRNIKGKWYHFDANGVAATGAQVINGQHLYFNEDGSQVKGSIVKNADGTFSKYKDSSGDLVVNEFFTTGDNVWYYAGANGKTVTGAQVINGQHLFFKEDGSQVKGDFVKNSDGTYSKYDAASGERLTNEFFTTGDNHWYYIGANGKTVTGEVKIGDDTYFFAKDGKQLKGQIVTTRSGRISYYFGDSGKKAISTWVEIQPGVFVFFDKNGLAYPPENMN

SEQ ID NO: 10 - protein, 1518 aa, Streptococcus salivarius
  REGION 1..1518: note = Wild type GTF corresponding to GTF 6855
MENKIHYKLHKVKKQWVTIAVASVALATVLGGLSVTTSSVSADETQDKTVTQSNSGTTASLVTSPEATKEADKRTNTKEADVLTPAKETNAVETATTTNTQATAEAATTATTADVAVAAVPNKEAVVTTDAPAVTTEKAEEQPATVKAEVVNTEVKAPEAALKDSEVEAALSLKNIKNIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYRTNEVYTSVRYGKDIMTANDTEGSKYSRTSGQVTLVANNPKLTLDQSAKLNVEMGKIHANQKYRALIVGTADGIKNFTSDADAIAAGYVKETDSNGVLTFGANDIKGYETFDMSGFVAVWVPVGASDDQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANTKSSGKDYQAKYGGEFLAELKAKYPEMFKVNMISTGKPIDDSVKLKQWKAEYFNGTNVLERGVGYVLSDEATGKYFTVTKDGNFIPLQLTGNEKVVTGFSNDGKGITYFGTSGTQAKSAFVTFNGNTYYFDARGHMVTNGEYSPNGKDVYRFLPNGIMLSNAFYVDANGNTYLYNSKGQMYKGGYTKFDVTETDKDGKESKVVKFRYFTNEGVMAKGVTVIDGFTQYFGEDGFQAKDKLVTFKGKTYYFDAHTGNAIKDTWRNINGKWYHFDANGVAATGAQVINGQKLYFNEDGSQVKGGVVKNADGTYSKYKEGSGELVTNEFFTTDGNVWYYAGANGKTVTGAQVINGQHLYFNADGSQVKGGVVKNADGTYSKYDASTGERLTNEFFTTGDNNWYYIGANGKSVTGEVKIGDDTYFFAKDGKQVKGQTVSAGNGRISYYYGDSGKRAVSTWIEIQPGVYVYFDKNGIAYPPRVLN

SEQ ID NO: 11 - protein, 1423 aa, Leuconostoc pseudomesenteroides
  source 1..1423: note = 0768 gtf truncated wildtype protein A
DASVATTVNPNLDDTQADNTNIQTPTDQNDESKDTTPKVETGDTTNSQSTEAQETTAQTNNDVETPQNSDAAIETGLLTTNNQIRYVNPDGTVLTGAYKTINGNTYYFDDDSGVALVGLHKIGDTLKGFSLNGVQVKGDYLTAANGDKYYFDSNGNAVSGVQQINGKTYYFDSTGKLMKGYTAVLNGVVTFFNSTTGEADNTDASTIKTGVTIDNSDYTVHNAAYDNTAASFDNINGYLTAESWYRPKEILENGESWRPSTAEDKRPILITWQPDIVTEVNYLNMMAANGLLSINAPFTTASDLAIMNDAVRAVQKNIEMRISQEKSTDWLKALMTQFINTQPQWNEVSESPSNDHLQGGALTYVNSPLTPDANSNFRLLNRTPTNQSGTTRYDTDKSKGGFELLLANDVDNSNPVVQAEQLNWLYYLMNFGSITANDPTANFDGIRVDAVDNVDADLLQIASDYFKLAYGTSLSDTNANQHLSILEDWSANDAEYMSKTGSNQLTMDTYTQQQLLFSLTKQVGNRADMRRFLEYFMINRANDSTENVATPNYSFVRAHDSEVQTVIATIIKDLHPDVVNSLAPTQAQLEEAFAVYNADMNRVDKQYTQYNMPSAYAMLLTNKDTIPRVYYGDLYTDDGEYMGTQTPYYDAIVNLLQSRVKYVAGGQSMAVDQHDILTSVRYGKNLADANATSDDLTSINSGIGVIVSNNPNLSLASGETVVLHMGIAHANQVYREILETTDNGIANNTDIFKTTDSNGDLIFTASEIHGYSNVQVSGFLSVWAPKDATDDQDVRTAASESTSNDGNTLHSNAALDSNLIYEGFSNFQSTPQSESEFANVKIAANVNLFKSWGVTSFQMAPQYRSSTDTSFLDSIIQNGYAFTDRYDLGFETPTKYGTDQQLRDAIKALHANGIQAMADFVPDQIYNLPQTELVSVSRTDSLGNQSANSNAANVLYVSHTVGGGEYQSKYGGEFLAIIKSKYPSLFKTIQVSTGLPIDDSTKIKEWSAKYFNGSNIQGRGFGYVLSDGGTQNYFKVISNSTDDDFLPNQLTGKPTMTGFEQTSKGIVYYSKSGIQAKNQFVKDDVSGNYYYFNKNGLMTVGSKTINGKNYMFLPNGVELRGSFLQTADGTVNYYATNGAQVQDSYVTDTEGNSYYFDGDGEMVTGTYTVDGHAQYFDVNGVQTKGAIITLGGVQRYYQAGNGNLATNQYVSYNNSWYYANTKGELVTGVQSINGNVQYFASNGQQIKGQIVVTGNQKSYYDANTGNLIKNDFLTPDQGKTWYYADQDGNLVVGAQEVNGHKLYFDDNGIQIKDQIISNDGQQYYYQGGNGDLVTNRYISYNDSWYYADATGVLVTGQQIINGETQYFRTDGRQVKGQIIADGDKQHYYDADSGNLVKNNFVTVDQGKTWYYADQDGNLSLVDR

SEQ ID NO: 12 - protein, 1375 aa, Leuconostoc pseudomesenteroides
  source 1..1375: note = 0768 gtf truncated wildtype protein B
STEAQETTAQTNNDVETPQNSDAAIETGLLTTNNQIRYVNPDGTVLTGAYKTINGNTYYFDDDSGVALVGLHKIGDTLKGFSLNGVQVKGDYLTAANGDKYYFDSNGNAVSGVQQINGKTYYFDSTGKLMKGYTAVLNGVVTFFNSTTGEADNTDASTIKTGVTIDNSDYTVHNAAYDNTAASFDNINGYLTAESWYRPKEILENGESWRPSTAEDKRPILITWQPDIVTEVNYLNMMAANGLLSINAPFTTASDLAIMNDAVRAVQKNIEMRISQEKSTDWLKALMTQFINTQPQWNEVSESPSNDHLQGGALTYVNSPLTPDANSNFRLLNRTPTNQSGTTRYDTDKSKGGFELLLANDVDNSNPVVQAEQLNWLYYLMNFGSITANDPTANFDGIRVDAVDNVDADLLQIASDYFKLAYGTSLSDTNANQHLSILEDWSANDAEYMSKTGSNQLTMDTYTQQQLLFSLTKQVGNRADMRRFLEYFMINRANDSTENVATPNYSFVRAHDSEVQTVIATIIKDLHPDVVNSLAPTQAQLEEAFAVYNADMNRVDKQYTQYNMPSAYAMLLTNKDTIPRVYYGDLYTDDGEYMGTQTPYYDAIVNLLQSRVKYVAGGQSMAVDQHDILTSVRYGKNLADANATSDDLTSINSGIGVIVSNNPNLSLASGETVVLHMGIAHANQVYREILETTDNGIANNTDIFKTTDSNGDLIFTASEIHGYSNVQVSGFLSVWAPKDATDDQDVRTAASESTSNDGNTLHSNAALDSNLIYEGFSNFQSTPQSESEFANVKIAANVNLFKSWGVTSFQMAPQYRSSTDTSFLDSIIQNGYAFTDRYDLGFETPTKYGTDQQLRDAIKALHANGIQAMADFVPDQIYNLPQTELVSVSRTDSLGNQSANSNAANVLYVSHTVGGGEYQSKYGGEFLAIIKSKYPSLFKTIQVSTGLPIDDSTKIKEWSAKYFNGSNIQGRGFGYVLSDGGTQNYFKVISNSTDDDFLPNQLTGKPTMTGFEQTSKGIVYYSKSGIQAKNQFVKDDVSGNYYYFNKNGLMTVGSKTINGKNYMFLPNGVELRGSFLQTADGTVNYYATNGAQVQDSYVTDTEGNSYYFDGDGEMVTGTYTVDGHAQYFDVNGVQTKGAIITLGGVQRYYQAGNGNLATNQYVSYNNSWYYANTKGELVTGVQSINGNVQYFASNGQQIKGQIVVTGNQKSYYDANTGNLIKNDFLTPDQGKTWYYADQDGNLVVGAQEVNGHKLYFDDNGIQIKDQIISNDGQQYYYQGGNGDLVTNRYISYNDSWYYADATGVLVTGQQIINGETQYFRTDGRQVKGQIIADGDKQHYYDADSGNLVKNNFVTVDQGKTWYYADQDGNLSLVDR

SEQ ID NO: 13 - protein, 1392 aa, Streptococcus salivarius
INGKQYYVNSDGSVRKNFVFEQDGKSYYFDAETGALATKSQDEFSTEPIKATVDFSSGNQLYKNDDKSLDQLDTFITADAWYRPKSILKDGKTWTASTEADKRPLLMVWWPDKSTQVNYLNYMQNQGLGAGSFSTNSSQESLDQAAKAVQTKIEERIAREGNTNWLRTSIDQFIKTQPGWNSSTENSSYDHLQGGQLLFNNSKGDTGNRTSHANSDYRLLNRTPTNQTGTRKYFKDNSIGGLEFLLANDIDNSNPAVQAEQLNWLHFMMNIGSIMANDPTANFDGLRVDALDNVDADLLQIASDYFKAAYGVDKSEANAIKHLSYLEAWSANDPYYNKDTKGAQLPIDNALRNALTNLLMRDKNTRMQLGDMTAFINSSLNPRGANDKNGERMANYIFTRAHDTEAQTIIQRIIRDRINPNLFGYNFTRDEIKKAFEIYNEDIDKAHKTYASYNLPSVYALMLTNKDSVTRVYYGDLYREDGHYMAKKTPYFDAIDTLLRARIKYVAGGQDMEVKKVGNDGLLTSVRYGKGANNRTDLGTSETRTQGMGVIMTNNYDFRLGSNETVTMNMGRAHRNQLYRPLLLTTKDGIATYLNDSDVPKKLLKRTDWNGNLTFNANDVFGVENVQVSGYLGVWVPYGAKANQDARTQPSNRANSDGQVYKSSAALDSQVMYEAFSNFQAFADDQPELYMNRVLAKNTDLLKAWGITSVGLPPQYVSSKDGTFLDSTIDNGYAFDDRYDMALSQNNKYGSLEDLLNVLRALHKDGIQAIADWVPDQIYNLPGKEVVNATRVNGYGNHQVGYQIIDQAYVANTRTDGTDYQGKYGGAFLDELKAKYPSIFNRVQISNGKQLPTNEKITKWSAKYFNGTNILGRGINYVLRDDKTNQYFNTSANSQLLPTPLRDTGAITSTQVFQRRGQDVYFLRDNQAVKNEFVQDGNGNWYYFGGDGKMTKGAQNINNKDYYFFDNGIQLRNALRRASNGYTYYYGLDGAMVKNAFVDFDDKHKQVRAFTTQGTMVVGNLHWSGHHFYFDRETGIQAKDRVVRTDDGKLHYYVAETGDMGRNVFATDSSTGKRYYFDADGNTVTGSRVIDGKTYYFNQDGSVGTAYSNRADSIIFENGKARYITPAGEIGRSIFVYNPETKAWNYFDKEGNRVTGRQYIDGHLYYFKEDGSQAKGEIIEENGVKYYYEPESGILASGRYLQVGDDQWMYFKQDGSLAIGQVRADHGYLKYFDKNGIQVKGKTVVEDGKTYYYDAHSGALVTSSFAEIAPNQWSYFNTDGQALKGKWTINGKEYYFDENGIQYKGKAIKVGSRYKYYDENDGQPVTNRFAQIEPNVWAYFGADGYAVTGEQVINGQHLYFDQSGRQVKGAYVTVNGQRRYYDANTGEYVPGR

(19) United States
(12) Patent Application Publication
(10) Pub. No.: US 2024/0108021 A1
(43) Pub. Date: Apr. 4, 2024

(54) MODIFICATION OF STEVIOL GLYCOSIDES BY GLUCOSYLTRANSFERASE ENZYMES

(71) Applicant: DuPont Nutrition Biosciences ApS, Kongens Lyngby (DK)

(72) Inventors: Karina Hansen Kjaer, Kongens Lyngby (DK); Slavko Kralj, OEGSTGEEST (NL)

(21) Appl. No.: 18/474,373

(22) Filed: Sep. 26, 2023

Related U.S. Application Data
(60) Provisional application No. 63/377,619, filed on Sep. 29, 2022.

(51) Int. Cl. A23C 9/12; A23C 9/13; A23G 9/34; A23G 9/36; A23L 27/30; C12P 19/18; C12P 19/56

(52) U.S. Cl. CPC A23C 9/1216; A23C 9/1307; A23G 9/34; A23G 9/363; A23L 27/36; C12P 19/18; C12P 19/56

(57) ABSTRACT

Disclosed herein are methods of glucosylating a steviol glycoside. These methods can comprise providing a composition that comprises at least (i) water, (ii) sucrose, (iii) stevioside or rebaudioside A, and (iv) a glucosyltransferase enzyme that synthesizes alpha-1,6-glucan or a glucosyltransferase enzyme that synthesizes alpha-1,3-glucan, wherein at least one glucosylated form of the stevioside or rebaudioside A is produced in the composition. Such a composition can be a food product/precursor, for instance.

This application claims the benefit of U.S. Provisional Appl. No. 63/377,619 (filed Sep. 29, 2022), which is incorporated herein by reference in its entirety.

### FIELD

The present disclosure is in the field of polysaccharides and food products. For example, the disclosure pertains to producing alpha-glucan in situ in food and food precursors.

### REFERENCE TO SEQUENCE LISTING SUBMITTED ELECTRONICALLY

The official copy of the sequence listing is submitted electronically via EFS-Web as a file named NB41982USNP_SequenceListing.xml created on Sep. 25, 2023 and having a size of about 35 kilobytes, and is filed concurrently with the specification. The sequence listing contained in this file is part of the specification and is incorporated herein by reference in its entirety.

### BACKGROUND

Texture is a key quality and value parameter for a range of food products. Today, stabilizers such as starch, carboxymethyl cellulose (CMC), cellulose, guar gum, pectin, xanthan and several others are common additives to food products for enhancing texture. Texture relates to consumer eating sensation and heavily impacts consumer perception.

A disadvantage of added stabilizers is that they can increase the total carbohydrate level and caloric content of food and often require special handling during processing. Plant-based or non-dairy protein food alternatives, such as soy, almond, pea, bean, rice or oat-based milks or fresh fermented products, are one of the fastest growing segments in all food product categories worldwide (2016, Makinen et al., Crit. Rev. Food Sci. Nutr. 56:339-349; Sethi et al., 2016, J. Food Sci. Technol. 53:3408-3423). However, most plant-based proteins lack a functional balance (Sethi et al., ibid.). Consequently, most plant-based, dairy-free products contain additives, such as emulsifiers, thickening agents and other stabilizers, which are in contradiction to the demand for a clean label (2017, Asioli et al., Food Res. Int. 99:58-71; Makinen et al., ibid.).

As an example of issues that can be raised by added stabilizers, fresh fermented products containing added starch can require special handling during processing so as to not lose the texture created by the starch through shear forces. In addition, it is well known that added starch can negatively impact products in several ways. First, starch diminishes the “shininess” of yogurt or plant-based fresh fermented products, negatively impacting consumer visual perception. Moreover, added starch often leads to undesirable sensory dryness of yogurt.

### SUMMARY

In one embodiment, the present disclosure concerns a method of glucosylating a steviol glycoside, the method comprising: providing a composition that comprises at least water, sucrose, a steviol glycoside, and a glucosyltransferase enzyme, wherein the steviol glycoside comprises stevioside or rebaudioside A, and wherein the glucosyltransferase enzyme is selected from: (i) a glucosyltransferase enzyme that synthesizes alpha-1,6-glucan, wherein at least about 50% of the glycosidic linkages of the alpha-1,6-glucan are alpha-1,6 linkages, and/or (ii) a glucosyltransferase enzyme that synthesizes alpha-1,3-glucan, wherein at least about 50% of the glycosidic linkages of the alpha-1,3-glucan are alpha-1,3 linkages, wherein at least one glucosylated form of the steviol glycoside (glucosylated steviol glycoside) is produced in the composition, and typically wherein at least one alpha-glucan is produced in the composition.

In another embodiment, the present disclosure concerns a composition, or a glucosylated steviol glycoside, produced by a method herein.

In another embodiment, the present disclosure concerns a composition comprising a glucosylated steviol glycoside, wherein the glucosylated steviol glycoside is produced by contacting a steviol glycoside with a glucosyltransferase enzyme in the presence of at least water and sucrose, wherein the steviol glycoside comprises stevioside or rebaudioside A, and wherein the glucosyltransferase enzyme is selected from: (i) a glucosyltransferase enzyme that synthesizes alpha-1,6-glucan, wherein at least about 50% of the glycosidic linkages of the alpha-1,6-glucan are alpha-1,6 linkages, and/or (ii) a glucosyltransferase enzyme that synthesizes alpha-1,3-glucan, wherein at least about 50% of the glycosidic linkages of the alpha-1,3-glucan are alpha-1,3 linkages, optionally wherein the composition is a food product/precursor.

### BRIEF DESCRIPTION OF THE DRAWINGS AND SEQUENCES

FIG. 1: Sugar content in YO-MIX 410 yogurt samples. Refer to Example 1.

FIG. 2: The apparent viscosity of YO-MIX 410 yogurt samples extracted at a shear rate of 11.7 Hz, which provides a measurement of product thickness. Refer to Example 1.

FIG. 3: The apparent viscosity of YO-MIX 410 yogurt samples extracted at a shear rate of 249 Hz, which provides a measurement of product mouthfeel. Refer to Example 1.

FIG. 4: Flow curves are shown that resulted from rotational rheological tests. Refer to Example 2.

FIG. 5: Sugar content of YO-MIX PRIME 900, YO-MIX 410, YO-MIX T42, YO-MIX M01 and YO-MIX 863 yogurt samples treated with various GTF enzyme regimens. Refer to Example 2.

FIGS. 6A-E: Spider plot of sensory data measured for YO-MIX 863 (FIG. 6A), YO-MIX PRIME 900 (FIG. 6B), YO-MIX 410 (FIG. 6C), YO-MIX T42 (FIG. 6D) and YO-MIX M01 (FIG. 6E) yogurt samples having 3% starch or treatment with vGTFJ alone (0:100% [GTF 0768: vGTFJ]) or both vGTFJ and GTF 0768 (90:10% [GTF 0768: vGTFJ]). Refer to Example 2.

FIG. 7: The apparent sweetness of YO-MIX 863, YO-MIX PRIME 900, YO-MIX 410, YO-MIX T42 and YO-MIX M01 yogurt samples treated with vGTFJ alone (0:100% [GTF 0768: vGTFJ]) or both vGTFJ and GTF 0768 (90:10% [GTF 0768: vGTFJ]), relative to the 3% starch samples (not enzyme-treated). Refer to Example 2.

FIG. 8: Sugar content in YO-MIX 410 yogurts treated with glucosyltransferase enzymes (vGTFJ, GTF 0768) and/or lactase/transgalactosidase enzymes. Refer to Example 3.

FIG. 9: The apparent viscosity of YO-MIX 410 yogurt samples treated with glucosyltransferase enzymes (vGTFJ, GTF 0768) and/or lactase/transgalactosidase enzymes, as measured by extraction at a shear rate of 11.7 Hz, which provides a measurement of product thickness. Refer to Example 3.

FIG. 10: The apparent viscosity of YO-MIX 410 yogurt samples treated with glucosyltransferase enzymes (vGTFJ, GTF 0768) and/or lactase/transgalactosidase enzymes, as measured by extraction at a shear rate of 249 Hz, which provides a measurement of mouthfeel. Refer to Example 3.

FIG. 11: Sugar content of substrates A-E with or without treatment with GTF 0974, GTF 0768, or v2GTFJ. Refer to Example 4.

FIG. 12: For each of substrates A-E with or without treatment with GTF 0974, GTF 0768, or v2GTFJ, bar height indicates percent sucrose reduction as compared to substrate reference as initially prepared (e.g., prior to NURICA enzyme incubation for forming substrate C, and prior to TGO enzyme incubation for forming substrate E). The interior of each bar shows the relative oligosaccharide and polysaccharide content (dry weight basis) as a percentage of the total content of oligosaccharides and polysaccharides. Refer to Example 4.

FIG. 13: The apparent viscosity of substrates A-E with or without treatment with GTF 0974, GTF 0768, or v2GTFJ, as measured by extraction at a shear rate of 11.7 Hz, which provides a measurement of product thickness. Refer to Example 4.

FIG. 14: The apparent viscosity of substrates A-E with or without treatment with GTF 0974, GTF 0768, or v2GTFJ, as measured by extraction at a shear rate of 249 Hz, which provides a measurement of mouthfeel. Refer to Example 4.

FIGS. 15A-B: Neutral beverages initially prepared without maltose (FIG. 15A) or with maltose (FIG. 15B), and then treated with GTF 0768, vGTFJ, or combinations thereof. For each resulting product, bar height indicates percent sucrose reduction as compared to reference (no GTF treatment) (reference is at 0%, so no bar). The interior of each bar shows the relative oligosaccharide and polysaccharide content (dry weight basis) as a percentage of the total content of oligosaccharides and polysaccharides formed in each GTF-treated product. Refer to Example 5.

FIG. 16: The apparent viscosity of neutral beverages, initially with or without maltose prior to GTF treatment, resulting from treatment with GTF 0768, vGTFJ, or a combination thereof, as measured by extraction at a shear rate of 11.7 Hz, which provides a measurement of product thickness. Refer to Example 5.

FIG. 17: The apparent viscosity of neutral beverages, initially with or without maltose prior to GTF treatment, resulting from treatment with GTF 0768, vGTFJ, or a combination thereof, as measured by extraction at a shear rate of 249 Hz, which provides a measurement of mouthfeel. Refer to Example 5.

FIG. 18: Viscosity of yogurt products over time (at 1, 7, 21, 35, or 63 days; bars shown in this order for each data set). Refer to Example 6.

FIG. 19: Sugar content of yogurt products over time (at 0, 14, 28, 42, or 56 days). Refer to Example 6.

FIG. 20: Melting profiles of ice cream samples. Refer to Example 7.

FIGS. 21A-C: Texture attributes (hardness [FIG. 21A], cohesiveness [FIG. 216], adhesiveness [FIG. 21C]) of ice cream samples. Refer to Example 7.

FIG. 22: Extensional rheometry (Hencky strain vs. time, at a rate of 1 mm/s) of sweet condensed milk samples. Refer to Example 8.

FIG. 23A: UHPLC results for a GTFJ reaction comprising rebaudioside A. Samples of the reaction were removed for analysis at reaction timepoints of 0 (#67), 15 (#68), 30 (#69), 60 (#70), 120 (#71) and 180 (#72) minutes. Refer to Example 10.

FIG. 23B: UHPLC results for a vGTFJ reaction comprising rebaudioside A. Samples of the reaction were removed for analysis at reaction timepoints of 0 (#172), 15 (#173), 30 (#174), 60 (#175), 120 (#176) and 180 (#177) minutes. Refer to Example 10.

FIG. 23C: UHPLC results for a GTF 0974 reaction comprising rebaudioside A. Samples of the reaction were removed for analysis at reaction timepoints of 0 (#258), 15 (#259), 30 (#260), 60 (#261), 120 (#262) and 180 (#263) minutes. Refer to Example 10.

FIG. 23D: UHPLC results for a GTF 0768 reaction comprising rebaudioside A. Samples of the reaction were removed for analysis at reaction timepoints of 0 (#215), 15 (#216), 30 (#217), 60 (#218), 120 (#219) and 180 (#220) minutes. Refer to Example 10.

FIG. 24A: UHPLC results for a GTFJ reaction comprising stevioside. Samples of the reaction were removed for analysis at reaction timepoints of 0 (#81), 15 (#82), 30 (#83), 60 (#84), 120 (#85) and 180 (#86) minutes. Refer to Example 10.

FIG. 24B: UHPLC results for a vGTFJ reaction comprising stevioside. Samples of the reaction were removed for analysis at reaction timepoints of 0 (#186), 15 (#187), 30 (#188), 60 (#189), 120 (#190) and 180 (#191) minutes. Refer to Example 10.

FIG. 24C: UHPLC results for a GTF 0974 reaction comprising stevioside. Samples of the reaction were removed for analysis at reaction timepoints of 0 (#134), 15 (#135), 30 (#136), 60 (#137), 120 (#138) and 180 (#139) minutes. Refer to Example 10.

FIG. 24D: UHPLC results for a GTF 0768 reaction comprising stevioside. Samples of the reaction were removed for analysis at reaction timepoints of 0 (#229), 15 (#230), 30 (#231), 60 (#232), 120 (#233) and 180 (#234) minutes. Refer to Example 10.

TABLE A
Summary of Protein SEQ ID Numbers

| | Protein |
|---|---|
| Description | SEQ ID NO. |
| GTF 0768. Mature form of Leuconostoc | 1 |
| pseudomesenteroides protein, but with two additional | (1449 aa) |
| N-terminal amino acids. | |
| GTF 0768. Mature, wild type form of L. | 2 |
| pseudomesenteroides protein (US2016/0122445). | (1447 aa) |
| vGTFJ. Variant of GTF 6855 (SEQ ID NO: 5 below). | 3 |
| | (1341 aa) |
| Variant of GTF 6855 (SEQ ID NO: 5 below). | 4 |
| | (1341 aa) |
| GTF 6855, derivable from Streptococcus salivarius | 5 |
| SK126. The first 178 amino acids of the protein are | (1341 aa) |
| deleted compared to GENBANK Acc. No. | |
| ZP_04061500.1; a start methionine is included. | |
| GTF 7527, derivable from Streptococcus salivarius. | 6 |
| The first 178 amino acids of the protein are deleted | (1341 aa) |
| compared to GENBANK Acc. No. CAA77900.1; a | |
| start methionine is included. | |
| GTF 2678, derivable from Streptococcus salivarius | 7 |
| K12. The first 188 amino acids of the protein are | (1341 aa) |
| deleted compared to GENBANK Acc. No. | |
| EJO16940.1; a start methionine is included. | |
| GTF 2919, derivable from Streptococcus salivarius | 8 |
| PS4. The first 92 amino acids of the protein are | (1340 aa) |
| deleted compared to GENBANK Acc. No. | |
| EIC80898.1; a start methionine is included. | |
| GTF 2765, derivable from unknown Streptococcus sp. | 9 |
| C150. The first 193 amino acids of the protein are | (1340 aa) |
| deleted compared to GENBANK Acc. No. | |
| ZP_08047301.1; a start methionine is included. | |
| Wild type GTF corresponding to GTF 6855, | 10 |
| Streptococcus salivarius SK126 (GENBANK Acc. No. | (1518 aa) |
| ZP_04061500.1). | |
| GTF 0768. N-terminal-truncated form A of the mature | 11 |
| form of L. pseudomesenteroides protein (SEQ ID | (1423 aa) |
| NO: 2). The first 24 amino acids of the protein are | |
| deleted compared to SEQ ID NO: 2. | |
| GTF 0768. N-terminal-truncated form B of the mature | 12 |
| form of L. pseudomesenteroides protein (SEQ ID | (1375 aa) |
| NO: 2). The first 72 amino acids of the protein are | |
| deleted compared to SEQ ID NO: 2. | |
| GTF 0974, derivable from Streptococcus salivarius | 13 |
| 57.I. The first 187 amino acids of the protein are | (1392 aa) |
| deleted compared to GENBANK Acc. No. | |
| AEJ53088.1. | |

### DETAILED DESCRIPTION

The disclosures of all cited patent and non-patent literature are incorporated herein by reference in their entirety.

Unless otherwise disclosed, the terms “a”, “an” and “the” as used herein are intended to encompass one or more (i.e., at least one) of a referenced feature.

Where present, all ranges are inclusive and combinable, except as otherwise noted. For example, when a range of “1 to 5” (i.e., 1-5) is recited, the recited range should be construed as including ranges “1 to 4”, “1 to 3”, “1-2”, “1-2 & 4-5”, “1-3 & 5”, and the like.

The terms “alpha-glucan”, “alpha-glucan polymer” and the like are used interchangeably herein. An alpha-glucan is a polymer comprising glucose monomeric units linked together by alpha-glycosidic linkages. In typical embodiments, an alpha-glucan herein comprises at least about 80%, 81%, 82%, 83%, 84%, 85%, 86%, 87%, 88%, 89%, 90%, 91%, 92%, 93%, 94%, 95%, 96%, 97%, 98%, 99%, or 100% alpha-glycosidic linkages. Examples of alpha-glucan polymers herein include graft copolymers as presently disclosed, as well as alpha-1,3-glucan and alpha-1,6-glucan.

The terms “alpha-1,3-glucan”, “poly alpha-1,3-glucan”, “alpha-1,3-glucan polymer” and the like are used interchangeably herein. Alpha-1,3-glucan is a polymer comprising glucose monomeric units linked together by glycosidic linkages, wherein at least about 50% of the glycosidic linkages are alpha-1,3. Alpha-1,3-glucan in certain embodiments comprises at least about 90% or 95% alpha-1,3 glycosidic linkages. Most or all of the other linkages in alpha-1,3-glucan herein typically are alpha-1,6, though some linkages may also be alpha-1,2 and/or alpha-1,4. Alpha-1,3-glucan as presently disclosed can characterize an alpha-1,3-glucan side chain herein. In some aspects, alpha-1,3-glucan can characterize an alpha-1,3-glucan “homopolymer”, which is alpha-1,3-glucan that is not part of a dextran-alpha-1,3-glucan copolymer.

The terms “dextran”, “dextran polymer”, “dextran molecule”, “alpha-1,6-glucan” and the like herein refer to a water-soluble alpha-glucan comprising at least 50%, 60%, 70%, 80%, or 90% alpha-1,6 glycosidic linkages (with the balance of the linkages typically being alpha-1,3). Enzymes capable of synthesizing dextran from sucrose may be described as “dextransucrases” (EC 2.4.1.5). A “substantially linear” (“mostly linear”, and like terms) dextran has 5% or less branches, before being modified herein to have with alpha-1,3-glucan side chains. A “linear” dextran has no branches, before being modified herein to have alpha-1,3-glucan side chains. Branches, if present prior to modification of dextran with alpha-1,3-glucan side chains, can be short, being one (pendant) to three glucose monomers in length. Yet, in some aspects, dextran can be “dendritic”, which is a branched structure emanating from a core in which there are chains (containing mostly or all alpha-1,6-linkages) that iteratively branch from each other (e.g., a chain can be a branch from another chain, which in turn is a branch from another chain, and so on). Yet, in still some aspects, dextran is not dendritic, but has a branch-on-branch structure that does not emanate from a core. Dextran as used in a glucosyltransferase reaction herein for alpha-1,3-glucan synthesis (to produce a dextran-alpha-1,3-glucan copolymer) can optionally be characterized as a “primer” or “acceptor”. In some aspects, dextran can characterize a dextran “homopolymer”, which is dextran that is not part of a dextran-alpha-1,3-glucan copolymer.

The term “copolymer” herein refers to a polymer comprising at least two different types of alpha-glucan, such as dextran and alpha-1,3-glucan.

The terms “graft copolymer”, “branched copolymer” and the like herein generally refer to a copolymer comprising a “backbone” (or “main chain”) and one or more side chains branching from the backbone. The side chains are structurally distinct from the backbone.

Examples of graft copolymers herein are “dextran-alpha-1,3-glucan graft copolymers” (and like terms) that comprise a backbone comprising dextran, and one or more side chains of alpha-1,3-glucan. A backbone in some aspects can itself be a branched dextran as disclosed herein; the addition of alpha-1,3-glucan side chains to such a backbone (thereby forming a graft copolymer herein) can be, for example, via enzymatic extension from non-reducing ends presented by short branches (alpha-1,2, -1,3, or -1,4 branch, each typically comprised of a single glucose monomer; i.e., pendant glucose). Short branches (that can be enzymatically extended into an alpha-1,3-glucan side chain) can be present on an otherwise linear or mostly linear dextran, or can be present on a branching dextran. In some aspects, alpha-1,3-glucan can also be synthesized from non-reducing ends of dextran main chains, such as in embodiments in which the dextran backbone is linear or mostly linear, or embodiments in which the dextran backbone is branching (e.g., dendritic, or not dendritic [branches do not emanate from a core] but has branch-on-branch structure); such alpha-1,3-glucan is not, technically-speaking, a side chain to the dextran, but rather an extension from the dextran main chain(s).

The percent branching in an alpha-glucan herein refers to that percentage of all the linkages in the alpha-glucan that represent branch points. For example, the percent of alpha-1,3 branching in an alpha-glucan herein refers to that percentage of all the linkages in the glucan that represent alpha-1,3 branch points. Except as otherwise noted, linkage percentages disclosed herein are based on the total linkages of a glucan, or the portion of a glucan for which a disclosure specifically regards.

The terms “linkage”, “glycosidic linkage”, “glycosidic bond” and the like refer to the covalent bonds connecting the sugar monomers within a saccharide compound (oligosaccharides and/or polysaccharides). Examples of glycosidic linkages include 1,6-alpha-D-glycosidic linkages (herein also referred to as “alpha-1,6” linkages), 1,3-alpha-D-glycosidic linkages (herein also referred to as “alpha-1,3” linkages), 1,4-alpha-D-glycosidic linkages (herein also referred to as “alpha-1,4” linkages), and 1,2-alpha-D-glycosidic linkages (herein also referred to as “alpha-1,2” linkages). The glycosidic linkages of a glucan polymer herein can also be referred to as “glucosidic linkages”. Herein, “alpha-D-glucose” is referred to as “glucose”.

The glycosidic linkage profile of an alpha-glucan herein can be determined using any method known in the art. For example, a linkage profile can be determined using methods using nuclear magnetic resonance (NMR) spectroscopy (e.g., ¹³C NMR or ¹H NMR). These and other methods that can be used are disclosed in, for example, Food Carbohydrates: Chemistry, Physical Properties, and Applications (S. W. Cui, Ed., Chapter 3, S. W. Cui, Structural Analysis of Polysaccharides, Taylor & Francis Group LLC, Boca Raton, F L, 2005), which is incorporated herein by reference.

The “molecular weight” of an alpha-glucan herein can be represented as weight-average molecular weight (Mw) or number-average molecular weight (Mn), the units of which are in Daltons (Da) or grams/mole. In some aspects, molecular weight can be represented as DPw (weight average degree of polymerization) or DPn (number average degree of polymerization). DPw and DPn can be calculated from the corresponding Mw or Mn, respectively, by dividing by the molar mass of one monomer unit M₁. In the case of glucan polymer, M₁=162.14. In some aspects, molecular weight can sometimes be provided as “DP” (degree of polymerization), which simply refers to the number of glucoses comprised within the alpha-glucan on an individual molecule basis. Various means are known in the art for calculating these various molecular weight measurements such as with high-pressure liquid chromatography (HPLC), size exclusion chromatography (SEC), or gel permeation chromatography (GPC).

The term “sucrose” herein refers to a non-reducing disaccharide composed of an alpha-D-glucose molecule and a beta-D-fructose molecule linked by an alpha-1,2-glycosidic bond. Sucrose is known commonly as table sugar. Sucrose can alternatively be referred to as “alpha-D-glucopyranosyl-(1→2)-beta-D-fructofuranoside”. “Alpha-D-glucopyranosyl” and “glucosyl” are used interchangeably herein.

The terms “sugar” or “sugars”, unless used to specifically refer to sucrose, refer to any monosaccharide, disaccharide, or oligosaccharide (e.g., ranging from DP3 to DP4, DP5, DP6, DP7, DP8, DP9, DP10, DP12, DP14, DP15, DP16, DP18, or DP20), such as those disclosed herein. Sugars herein typically are water-soluble.

The terms “glucosyltransferase”, “glucosyltransferase enzyme”, “GTF”, “glucansucrase” and the like are used interchangeably herein. The activity of a glucosyltransferase herein catalyzes the reaction of the substrate sucrose to make the products alpha-glucan and fructose. Other products (by-products) of a GTF reaction can include glucose, various soluble gluco-oligosaccharides, and leucrose. Wild type forms of glucosyltransferase enzymes generally contain (in the N-terminal to C-terminal direction) a signal peptide (which is typically removed by cleavage processes), a variable domain, a catalytic domain, and a glucan-binding domain. A glucosyltransferase herein is classified under the glycoside hydrolase family 70 (GH70) according to the CAZy (Carbohydrate-Active EnZymes) database (Cantarel et al., Nucleic Acids Res. 37:D233-238, 2009). The term “dextransucrase” (and like terms) can optionally be used to characterize a glucosyltransferase enzyme that produces dextran.

The term “glucosyltransferase catalytic domain” herein refers to the domain of a glucosyltransferase enzyme that provides alpha-glucan-synthesizing activity to a glucosyltransferase enzyme. A glucosyltransferase catalytic domain typically does not require the presence of any other domains to have this activity.

The terms “enzymatic reaction”, “glucosyltransferase reaction”, “glucan synthesis reaction”, “reaction composition”, “reaction formulation” and the like are used interchangeably herein and generally refer to a reaction that initially comprises water, sucrose, at least one active glucosyltransferase enzyme, and optionally other components. Components that can be further present in a glucosyltransferase reaction typically after it has commenced include fructose, glucose, leucrose, soluble gluco-oligosaccharides (e.g., DP2-DP7) (such may be considered as products or by-products, depending on the glucosyltransferase used), and/or insoluble alpha-glucan product(s) of DP8 or higher. It would be understood that certain glucan products, such as alpha-1,3-glucan with a degree of polymerization (DP) of at least 8 or 9, are water-insoluble and thus not dissolved in a glucan synthesis reaction. The term “under suitable reaction conditions” as used herein refers to reaction conditions that support conversion of sucrose to alpha-glucan product(s) via glucosyltransferase enzyme activity. It is during such a reaction that glucosyl groups originally derived from the input sucrose are enzymatically transferred and used in alpha-glucan polymer synthesis; glucosyl groups as involved in this process can thus optionally be referred to as the glucosyl component or moiety (or like terms) of a glucosyltransferase reaction.

The “yield” of an alpha-glucan product in a glucosyltransferase reaction in some aspects herein represents the molar yield based on the converted sucrose. The molar yield of an alpha-glucan product can be calculated based on the moles of the alpha-glucan product divided by the moles of the sucrose converted. Moles of converted sucrose can be calculated as follows: (mass of initial sucrose−mass of final sucrose)/molecular weight of sucrose [342 g/mol]. This molar yield calculation can be considered as a measure of selectivity of the reaction toward the alpha-glucan. In some aspects, the “yield” of an alpha-glucan product in a glucosyltransferase reaction can be based on the glucosyl component of the reaction. Such a yield (yield based on glucosyl) can be measured using the following formula:

Alpha-Glucan Yield=((IS/2−(FS/2+LE/2+GL+SO))/(IS/2−FS/2))×100%.

The fructose balance of a glucosyltransferase reaction can be measured to ensure that HPLC data, if applicable, are not out of range (90-110% is considered acceptable). Fructose balance can be measured using the following formula:

Fructose Balance=((180/342×(FS+LE)+FR)/(180/342×IS))×100%.

In the above two formulae, IS is [Initial Sucrose], FS is [Final Sucrose], LE is [Leucrose], GL is [Glucose], SO is [Soluble Oligomers] (gluco-oligosaccharides), and FR is [Fructose]; the concentrations of each foregoing substrate/product provided in double brackets are in units of grams/L and as measured by HPLC, for example.

The term “in situ” as used herein characterizes a glucosyltransferase reaction(s) that occurs inside a food product or precursor thereof and thereby produces alpha-glucan within the food product itself (or precursor). Such produced alpha-glucan (e.g., graft copolymer, alpha-1,3-glucan, and/or alpha-1,6-glucan) can be soluble or insoluble. While an alpha-1,3-glucan product is typically insoluble and an alpha-1,6-glucan product is typically soluble, a graft copolymer product can either be soluble or insoluble, in a food product/precursor herein. In situ production of alpha-glucan in a food product/precursor typically substitutes for adding alpha-glucan herein as an ingredient in food, though such addition can be performed if desired (e.g., to supplement the alpha-glucan produced in situ).

The terms “percent by volume”, “volume percent”, “vol %”, “v/v %” and the like are used interchangeably herein. The percent by volume of a solute in a solution can be determined using the formula: [(volume of solute)/(volume of solution)]×100%.

The terms “percent by weight”, “weight percentage (wt %)”, “weight-weight percentage (% w/w)” and the like are used interchangeably herein. Percent by weight refers to the percentage of a material on a mass basis as it is comprised in a composition, mixture, or solution.

The terms “weight/volume percent”, “w/v %” and the like are used interchangeably herein. Weight/volume percent can be calculated as: ((mass [g] of material)/(total volume [mL] of the material plus the liquid in which the material is placed))×100%. The material can be insoluble in the liquid (i.e., be a solid phase in a liquid phase, such as with a dispersion), or soluble in the liquid (i.e., be a solute dissolved in the liquid).

The terms “ingestible product” and “ingestible composition” are used interchangeably herein, and refer to any substance that, either alone or together with another substance, may be taken orally (i.e., by mouth), whether intended for consumption or not. Thus, an ingestible product includes food/beverage products. “Food/beverage products” refer to any edible product intended for consumption (e.g., for nutritional purposes) by humans or animals, including solids, semi-solids, or liquids. A “food” herein can optionally be referred to as a “foodstuff”, “food product”, or other like term, for example. Herein, unless otherwise disclosed, a beverage or other ingestible liquid is an example of a food product. While the present disclosure generally regards food and food precursors that are by definition intended for ingestion or eventual ingestion (food precursor first made into food before being eaten), the disclosure likewise regards other ingestible products (e.g., supplement, nutraceutical, pharmaceutical product) comprising in situ-produced alpha-glucan. A food precursor herein can be (i) a food as it exists before one or more processing steps (e.g., fermentation, aging, cooling/freezing, heating, baking, mixing) that render it to be a food product intended for direct consumption, and/or (ii) an ingredient for use in preparing a food product, for example. In some aspects, a food precursor can characterize a food product or ingredient as it exists before treatment with one or more GTF enzymes in a method herein.

The term “texture” as used herein in reference to a food product/precursor herein means the thickness of the food product/precursor and/or sensory perception of the food product/precursor by, for example, vision, touch, or oral/taste processing. An “improvement” in texture means an increase in thickness and/or an increase in the sensory perception. Unless otherwise noted, as used herein the “thickness” of a food product/precursor means the apparent viscosity extracted at shear rate of about 10-13 Hz (e.g., ˜11.7 Hz) during a rheological analysis; an increase in apparent viscosity at such a shear rate indicates an increase in thickness. The apparent viscosity extracted at shear rate of about 230-270 Hz (e.g., ˜249 Hz) during a rheological analysis is correlated to “mouthfeel”; an increase in apparent viscosity at such a shear rate indicates an increase in mouthfeel.

“Dairy product/precursor” and like terms herein refer to a food product/precursor that contains milk and/or is made from milk. In some aspects, a dairy product/precursor contains at least about 2.5, 5, or 10 wt % milk or milk solids.

“Lactase-treated milk/dairy” and like terms herein refer to milk/dairy products or precursors treated with one or more lactase enzymes to reduce the amount of lactose sugar therein.

“Reduced lactose milk/dairy” and like terms herein refer to milk/dairy products or precursors in which the weight percentage of lactose is about 2% or less, for example. “Lactose-free milk/dairy” and like terms herein refer to milk/dairy products or precursors in which the weight percentage of lactose is about 0.5 wt % or less, for example.

“Yogurt”, “dairy-based yogurt”, “fermented dairy product” and like terms herein generally refer to a dairy food/beverage produced by acidifying lactic fermentation of a dairy substrate such as milk. Such a product can optionally contain secondary ingredients such as fruits, vegetables, sugars, flavors, etc.

“Flour” and like terms herein refer to powder made by grinding (milling) grains/cereals, roots/tubers, beans/legumes, or nuts/seeds, for example. Typically, the material that is ground into flour is entered into the grinding process in raw, dried form. A flour herein that is made from grain can optionally be referred to as a “grain flour”. “Meal” and other like terms herein refer to a substance that is similar to flour, but with a grain or particle size that is larger/coarser. A meal is not ground/milled as finely as flour. A meal herein that is made from grain can optionally be referred to as a “grain meal”. Flour and meal are generally used as ingredients in various food products. Flour and meal produced from a grain can optionally be characterized as grain derivatives herein.

“Dough”, “food dough” and like terms herein refer to a mixture comprising at least (i) flour and/or meal and (ii) a liquid (e.g., water or milk), and typically is in a suitable form (stiff/firm) for kneading or rolling. A dough can optionally be referred to with reference to the grain, grain derivative, or other material from which it was derived (e.g., wheat dough, wheat flour dough, corn flour dough, cornmeal dough). Since dough typically is not eaten as a food prior to further processing (e.g., baking), dough can optionally be characterized as a “food precursor”.

A “baked food” (and like terms) herein refers to a food that has been baked during its preparation. Baking herein refers to a process of applying dry heat to a food/food precursor for a period of time during preparation of the food. In general, baking is conducted in an enclosed (typically confined) space such as within an oven. Bread is an example of a food for which its preparation process comprises baking.

An “extruded food” (and like terms) herein refers to a food that has been extruded during its preparation. Food extrusion is a process by which a mix of ingredients (e.g., dough) is forced through an opening in a perforated device (e.g., plate or die), which typically is specifically designed for the food being extruded. After this step, an extruded food typically is then cut to a particular size.

The terms “dietary fiber”, “glucan fiber” and the like herein refer to an alpha-glucan that is indigestible and/or that does not increase blood-glucose levels when enterally administered to a mammal. In general, a dietary fiber herein is not significantly hydrolyzed by endogenous enzymes in the upper gastrointestinal tract of mammals such as humans.

“Fermentation” and like terms herein as applied to food product/precursor refer to the conversion of carbohydrates in a food product/precursor into alcohol(s) and/or acid(s) through the action of one or more microorganisms (e.g., bacteria, yeast).

A composition herein that is “dry” or “dried” typically has less than 5, 4, 3, 2, 1, 0.5, or 0.1 wt % water comprised therein.

The terms “aqueous liquid”, “aqueous fluid”, “aqueous conditions”, “aqueous setting”, “aqueous system” and the like as used herein can refer to water or an aqueous solution. An “aqueous solution” herein can comprise one or more dissolved salts, where the maximal total salt concentration can be about 3.5 wt % in some embodiments. Although aqueous liquids herein typically comprise water as the only solvent in the liquid, an aqueous liquid can optionally comprise one or more other solvents (e.g., polar organic solvent) that are miscible in water. Thus, an aqueous solution can comprise a solvent having at least about 10 wt % water.

An “aqueous composition” herein has a liquid component that comprises about, or at least about, 10, 20, 30, 40, 50, 60, 70, 80, 90, 95, 99, or 100 wt % water, for example. Examples of aqueous compositions include mixtures, solutions, dispersions (e.g., suspensions, colloidal dispersions) and emulsions, for example.

Alpha-glucan in some aspects of the present disclosure can provide stability to a dispersion or emulsion of a food product/precursor. The “stability” (or the quality of being “stable”) of a dispersion or emulsion herein is, for example, the ability of dispersed particles of a dispersion, or liquid droplets dispersed in another liquid (emulsion), to remain dispersed (e.g., about, or at least about, 70, 75, 80, 85, 90, 95, 96, 97, 98, 99, or 100 wt % of the particles of the dispersion or liquid droplets of the emulsion are in a dispersed state) for a period of about, or at least about, 2, 4, 6, 9, 12, 18, 24, 30, or 36 months following initial preparation of the dispersion or emulsion. A stable dispersion or emulsion can resist total creaming, sedimentation, flocculation, and/or coalescence of dispersed/emulsified material, for example.

An alpha-glucan herein that is “insoluble”, “aqueous-insoluble”, “water-insoluble” (and like terms) herein does not dissolve (or does not appreciably dissolve) in water or other aqueous conditions, optionally where the aqueous conditions are at a pH of 4-9 (e.g., pH 6-8) and/or a temperature of about 1 to 130° C. (e.g., 20-25° C.). In some aspects, less than 1.0 gram (e.g., no detectable amount) of an aqueous-insoluble alpha-glucan dissolves in 1000 milliliters of such aqueous conditions (e.g., water at 23° C.). In contrast, an alpha-glucan that is “soluble”, “aqueous-soluble”, “water-soluble” and the like appreciably dissolves under the above aqueous conditions.

The term “viscosity” as used herein refers to the resistance of a food product/precursor to deformation at a given rate. Viscosity may also be defined as a measure of the extent to which a fluid (aqueous or non-aqueous) resists a force tending to cause it to flow. Furthermore, viscosity can be defined as the shear stress resulting from an applied shear rate. Both dynamic and kinematic viscosity are meant by the term viscosity, as both parameters are directly correlated through the density of a food product/precursor. Various units of viscosity that can be used herein include centipoise (cP, cps) and Pascal-second (Pa.$), for example. A centipoise is one one-hundredth of a poise; one poise is equal to 0.100 kgm⁻¹·s⁻¹.

The terms “polynucleotide”, “polynucleotide sequence”, “nucleic acid molecule” and the like are used interchangeably herein. These terms encompass nucleotide sequences and the like. A polynucleotide may be a polymer of DNA or RNA that is single- or double-stranded, that optionally contains synthetic, non-natural or altered nucleotide bases. A polynucleotide may be comprised of one or more segments of cDNA, genomic DNA, synthetic DNA, or mixtures thereof.

The term “gene” as used herein refers to a DNA polynucleotide sequence that expresses an RNA (RNA is transcribed from the DNA polynucleotide sequence) from a coding region, which RNA can be a messenger RNA (encoding a protein) or a non-protein-coding RNA. A gene may refer to the coding region alone, or may include regulatory sequences upstream and/or downstream to the coding region (e.g., promoters, 5′-untranslated regions, 3′-transcription terminator regions). A coding region encoding a protein can alternatively be referred to herein as an “open reading frame” (ORF). A gene that is “native” or “endogenous” refers to a gene as found in nature with its own regulatory sequences; such a gene is located in its natural location in the genome of a host cell. A “chimeric” gene refers to any gene that is not a native gene, comprising regulatory and coding sequences that are not found together in nature (i.e., the regulatory and coding regions are heterologous with each other). Accordingly, a chimeric gene may comprise regulatory sequences and coding sequences that are derived from different sources, or regulatory sequences and coding sequences derived from the same source, but arranged in a manner different than that found in nature. A “foreign” or “heterologous” gene can refer to a gene that is introduced into the host organism by gene transfer. Foreign/heterologous genes can comprise native genes inserted into a non-native organism, native genes introduced into a new location within the native host, or chimeric genes. Polynucleotide sequences in certain aspects disclosed herein are heterologous. A “transgene” is a gene that has been introduced into the genome by a gene delivery procedure (e.g., transformation). A “codon-optimized” open reading frame has its frequency of codon usage designed to mimic the frequency of preferred codon usage of the host cell.

As used herein, the term “polypeptide” is defined as a chain of amino acid residues, usually having a defined sequence. As used herein the term polypeptide is interchangeable with the terms “peptides” and “proteins”. Typical amino acids contained in polypeptides herein include (respective three- and one-letter codes shown parenthetically): alanine (Ala, A), arginine (Arg, R), asparagine (Asn, N), aspartic acid (Asp, D), cysteine (Cys, C), glutamic acid (Glu, E), glutamine (Gln, Q), glycine (Gly, G), histidine (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), valine (Val, V).

The term “heterologous” means not naturally found in the location of interest. For example, a heterologous gene can be one that is not naturally found in a host organism, but that is introduced into the host organism by gene transfer. As another example, a nucleic acid molecule that is present in a chimeric gene can be characterized as being heterologous, as such a nucleic acid molecule is not naturally associated with the other segments of the chimeric gene (e.g., a promoter can be heterologous to a coding sequence).

A “non-native” amino acid sequence or polynucleotide sequence comprised in a cell or organism herein does not occur in a native (natural) counterpart of such cell or organism, or does not occur in nature. Such an amino acid sequence or polynucleotide sequence can also be referred to as being heterologous to the cell or organism.

“Regulatory sequences” as used herein refer to nucleotide sequences located upstream of a gene's transcription start site (e.g., promoter), 5′ untranslated regions, introns, and 3′ non-coding regions, and which may influence the transcription, processing or stability, and/or translation of an RNA transcribed from the gene. Regulatory sequences herein may include promoters, enhancers, silencers, 5′ untranslated leader sequences, introns, polyadenylation recognition sequences, RNA processing sites, effector binding sites, stem-loop structures, and other elements involved in regulation of gene expression. One or more regulatory elements herein may be heterologous to a coding region herein.

A “promoter” as used herein refers to a DNA sequence capable of controlling the transcription of RNA from a gene. In general, a promoter sequence is upstream of the transcription start site of a gene. Promoters may be derived in their entirety from a native gene, or be composed of different elements derived from different promoters found in nature, or even comprise synthetic DNA segments. Promoters that cause a gene to be expressed in a cell at most times under all circumstances are commonly referred to as “constitutive promoters”. A promoter may alternatively be inducible. One or more promoters herein may be heterologous to a coding region herein.

A “strong promoter” as used herein refers to a promoter that can direct a relatively large number of productive initiations per unit time, and/or is a promoter driving a higher level of gene transcription than the average transcription level of the genes in a cell.

The terms “3′ non-coding sequence”, “transcription terminator”, “terminator” and the like as used herein refer to DNA sequences located downstream of a coding sequence. This includes polyadenylation recognition sequences and other sequences encoding regulatory signals capable of affecting mRNA processing or gene expression.

The terms “upstream” and “downstream” as used herein with respect to polynucleotides refer to “5′ of” and “3′ of”, respectively.

The term “expression” as used herein refers to (i) transcription of RNA (e.g., mRNA or a non-protein-coding RNA) from a coding region, and/or (ii) translation of a polypeptide from mRNA. Expression of a coding region of a polynucleotide sequence can be up-regulated or down-regulated in certain embodiments.

The term “operably linked” as used herein refers to the association of two or more nucleic acid sequences such that the function of one is affected by the other. For example, a promoter is operably linked with a coding sequence when it is capable of affecting the expression of that coding sequence. That is, the coding sequence is under the transcriptional control of the promoter. A coding sequence can be operably linked to one (e.g., promoter) or more (e.g., promoter and terminator) regulatory sequences, for example.

The term “recombinant” when used herein to characterize a DNA sequence such as a plasmid, vector, or construct refers to an artificial combination of two otherwise separated segments of sequence, e.g., by chemical synthesis and/or by manipulation of isolated segments of nucleic acids by genetic engineering techniques.

The term “transformation” as used herein refers to the transfer of a nucleic acid molecule into a host organism or host cell by any method. A nucleic acid molecule that has been transformed into an organism/cell may be one that replicates autonomously in the organism/cell, or that integrates into the genome of the organism/cell, or that exists transiently in the cell without replicating or integrating. Non-limiting examples of nucleic acid molecules suitable for transformation are disclosed herein, such as plasmids and linear DNA molecules. Host organisms/cells herein containing a transforming nucleic acid sequence can be referred to as “transgenic”, “recombinant”, “transformed”, “engineered”, as a “transformant”, and/or as being “modified for exogenous gene expression”, for example.

The terms “sequence identity”, “identity” and the like as used herein with respect to polynucleotide or polypeptide sequences refer to the nucleic acid residues or amino acid residues in two sequences that are the same when aligned for maximum correspondence over a specified comparison window. Thus, “percentage of sequence identity”, “percent identity” and the like refer to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. It would be understood that, when calculating sequence identity between a DNA sequence and an RNA sequence, T residues of the DNA sequence align with, and can be considered “identical” with, U residues of the RNA sequence. For purposes of determining “percent complementarity” of first and second polynucleotides, one can obtain this by determining (i) the percent identity between the first polynucleotide and the complement sequence of the second polynucleotide (or vice versa), for example, and/or (ii) the percentage of bases between the first and second polynucleotides that would create canonical Watson and Crick base pairs.

Percent identity can be readily determined by any known method, including but not limited to those described in: 1) Computational Molecular Biology (Lesk, A. M., Ed.) Oxford University: NY (1988); 2) Biocomputing: Informatics and Genome Projects (Smith, D. W., Ed.) Academic: NY (1993); 3) Computer Analysis of Sequence Data, Part I (Griffin, A. M., and Griffin, H. G., Eds.) Humana: NJ (1994); 4) Sequence Analysis in Molecular Biology (von Heinje, G., Ed.) Academic (1987); and 5) Sequence Analysis Primer (Gribskov, M. and Devereux, J., Eds.) Stockton: NY (1991), all of which are incorporated herein by reference.

Preferred methods for determining percent identity are designed to give the best match between the sequences tested. Methods of determining identity and similarity are codified in publicly available computer programs, for example. Sequence alignments and percent identity calculations can be performed using the MEGALIGN program of the LASERGENE bioinformatics computing suite (DNASTAR Inc., Madison, WI), for example. Multiple alignment of sequences can be performed, for example, using the Clustal method of alignment which encompasses several varieties of the algorithm including the Clustal V method of alignment (described by Higgins and Sharp, CABIOS. 5:151-153 (1989); Higgins, D. G. et al., Comput. Appl. Biosci., 8:189-191 (1992)) and found in the MEGALIGN v8.0 program of the LASERGENE bioinformatics computing suite (DNASTAR Inc.). For multiple alignments, the default values can correspond to GAP PENALTY=10 and GAP LENGTH PENALTY=10. Default parameters for pairwise alignments and calculation of percent identity of protein sequences using the Clustal method can be KTUPLE=1, GAP PENALTY=3, WINDOW=5 and DIAGONALS SAVED=5. For nucleic acids, these parameters can be KTUPLE=2, GAP PENALTY=5, WINDOW=4 and DIAGONALS SAVED=4. Additionally, the Clustal W method of alignment can be used (described by Higgins and Sharp, CABIOS. 5:151-153 (1989); Higgins, D. G. et al., Comput. Appl. Biosci. 8:189-191(1992); Thompson, J. D. et al, Nucleic Acids Research, 22 (22): 4673-4680, 1994) and found in the MEGALIGN v8.0 program of the LASERGENE bioinformatics computing suite (DNASTAR Inc.). Default parameters for multiple alignment (protein/nucleic acid) can be: GAP PENALTY=10/15, GAP LENGTH PENALTY=0.2/6.66, Delay Divergent Seqs(%)=30/30, DNA Transition Weight=0.5, Protein Weight Matrix=Gonnet Series, DNA Weight Matrix=IUB.

Various polypeptide amino acid sequences and polynucleotide sequences are disclosed herein as features of certain embodiments. Variants of these sequences that are at least about 70-85%, 85-90%, or 90%-95% identical to the sequences disclosed herein can be used or referenced. Alternatively, a variant amino acid sequence or polynucleotide sequence can have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identity with a sequence disclosed herein. A variant amino acid sequence or polynucleotide sequence herein has the same function/activity of the disclosed sequence, or at least about 80%, 81%, 82%, 83%, 84%, 85%, 86%, 87%, 88%, 89%, 90%, 91%, 92%, 93%, 94%, 95%, 96%, 97%, 98%, 99%, 99.5% of the function/activity of the disclosed sequence. Any polypeptide amino acid sequence disclosed herein not beginning with a methionine or valine can typically further comprise at least a start-methionine or start-valine at the N-terminus of the amino acid sequence. In contrast, any polypeptide amino acid sequence disclosed herein beginning with a methionine or valine can optionally lack such a methionine or valine residue. In some aspects, any polypeptide amino acid sequence disclosed herein beginning with a methionine or valine can instead have, respectively, a valine or methionine as the first amino acid residue.

The terms “aligns with”, “corresponds with”, and the like can be used interchangeably herein. Some aspects herein relate to a glucosyltransferase comprising at least one amino acid substitution at a position corresponding with at least one particular amino acid residue of SEQ ID NO:10. An amino acid position of a glucosyltransferase or subsequence thereof (e.g., catalytic domain or catalytic domain plus glucan-binding domains) (can refer to such an amino acid position or sequence as a “query” position or sequence) can be characterized to correspond with a particular amino acid residue of SEQ ID NO:10 (can refer to such an amino acid position or sequence as a “subject” position or sequence) if (1) the query sequence can be aligned with the subject sequence (e.g., where an alignment indicates that the query sequence and the subject sequence [or a subsequence of the subject sequence] are at least about 30%, 40%, 50%, 60%, 70%, 80%, or 90% identical), and (2) if the query amino acid position directly aligns with (directly lines up against) the subject amino acid position in the alignment of (1). In general, one can align a query amino acid sequence with a subject sequence (SEQ ID NO:10 or a subsequence of SEQ ID NO:10) using any alignment algorithm, tool and/or software described disclosed herein (e.g., BLASTP, ClustalW, ClustalV, Clustal-Omega, EMBOSS) to determine percent identity. Just for further example, one can align a query sequence with a subject sequence herein using the Needleman-Wunsch algorithm (Needleman and Wunsch, J. Mol. 48:443-453, 1970) as implemented in the Needle program of the European Molecular Biology Open Software Suite (EMBOSS [e.g., version 5.0.0 or later], Rice et al., Trends Genet. 16:276-277, 2000). The parameters of such an EMBOSS alignment can comprise, for example: gap open penalty of 10, gap extension penalty of 0.5, EBLOSUM62 (EMBOSS version of BLOSUM62) substitution matrix.

The numbering of particular amino acid residues of SEQ ID NO:10 herein (e.g., Tyr-185, Val-186, Leu-513, Gln-588, Phe-607, Ile-608, Lys-625, Arg-741, Val-1188, Lys-1327, Glu-1332, Asp-1418, Ala-1419, Ser-1420, Thr-1421, Arg-1424, Leu-1425, Thr-1431, Glu-1450) is with respect to the full-length amino acid sequence of SEQ ID NO:10. The first amino acid (i.e., position 1, Met-1) of SEQ ID NO:10 is at the start of the signal peptide. Unless otherwise disclosed, substitutions herein are in correspondence to the full-length amino acid sequence of SEQ ID NO:10 as reference sequence.

A “non-native glucosyltransferase” herein (“mutant”, “variant”, “modified” and like terms can likewise be used to describe such a glucosyltransferase) has at least one amino acid substitution at a position corresponding with a particular amino acid residue of SEQ ID NO:10 (SEQ ID NOs:3 and 4 are examples of a non-native GTF). Such at least one amino acid substitution typically is in place of the amino acid residue(s) that normally (natively) occurs at the same position in the native counterpart (parent) of the non-native glucosyltransferase (i.e., although SEQ ID NO:10 is used as a reference for position, an amino acid substitution herein is with respect to the native counterpart of a non-native glucosyltransferase) (considered another way, when aligning the sequence of a non-native glucosyltransferase with SEQ ID NO:10, determining whether a substitution exists at a particular position does not depend in-and-of-itself on the respective amino acid residue in SEQ ID NO:10, but rather depends on what amino acid exists at the subject position within the native counterpart of the non-native glucosyltransferase). The amino acid normally occurring at the relevant site in the native counterpart glucosyltransferase often (but not always) is the same as (or conserved with) the particular amino acid residue of SEQ ID NO:10 for which the alignment is made. A non-native glucosyltransferase optionally can have other amino acid changes (mutations, deletions, and/or insertions) relative to its native counterpart sequence.

The term “isolated” means a substance (or process) in a form or environment that does not occur in nature. A non-limiting example of an isolated substance includes any non-naturally occurring substance such as a food product, food precursor, or graft copolymer herein (as well as enzymatic reactions used to prepare these materials). It is believed that the embodiments disclosed herein are synthetic/man-made (could not have been made except for human intervention/involvement), and/or have properties that are not naturally occurring.

The term “increased” as used herein can refer to a quantity or activity that is at least about 1%, 2%, 3%, 4%, 5%, 6%, 7%, 8%, 9%, 10%, 11%, 12%, 13%, 14%, 15%, 16%, 17%, 18%, 19%, 20%, 50%, 100%, or 200% more than the quantity or activity for which the increased quantity or activity is being compared. The terms “increased”, “elevated”, “enhanced”, “greater than”, “improved” and the like are used interchangeably herein.

Some embodiments of the present disclosure concern a method of producing a food product/precursor. Such a method can comprise:
(a) providing a food product or food precursor (“food product/precursor”) that comprises at least water and sucrose, and
(b) contacting the food product/precursor with at least:
   (i) a glucosyltransferase enzyme that synthesizes alpha-1,6-glucan, wherein at least about 50% of the glycosidic linkages of the alpha-1,6-glucan are alpha-1,6 linkages, and
   (ii) a glucosyltransferase enzyme that synthesizes alpha-1,3-glucan, wherein at least about 50% of the glycosidic linkages of the alpha-1,3-glucan are alpha-1,3 linkages,
wherein at least one alpha-glucan is produced in the food product/precursor, whereby the food product/food precursor, after step (b), optionally has one or more of the following features as compared to the food product/precursor before step (b):
(I) reduced sugar content,
(II) increased texture, such texture optionally comprising increased thickness and/or increased mouthfeel,
(III) improved physical appearance, and/or
(IV) increased stringiness or stretchability.

Step (b) of producing a food product/precursor can comprise contacting a food product/precursor with at least:
(i) a glucosyltransferase (GTF) enzyme that synthesizes alpha-1,6-glucan, wherein at least about 50% of the glycosidic linkages of the alpha-1,6-glucan are alpha-1,6 linkages, and
(ii) a GTF enzyme that synthesizes alpha-1,3-glucan, wherein at least about 50% of the glycosidic linkages of the alpha-1,3-glucan are alpha-1,3 linkages.

In some aspects, a GTF enzyme (dextransucrase) that synthesizes alpha-1,6-glucan herein can comprise an amino acid sequence that is about 100% identical to, or at least about 90%, 91%, 92%, 93%, 94%, 95%, 96%, 97%, 98%, 98.5%, 99%, or 99.5% identical to, SEQ ID NO:1, 2, 11, or 12 (GTF 0768), and have GTF activity. Yet, in some aspects, a GTF enzyme that synthesizes alpha-1,6-glucan can be as disclosed in any of U.S. Patent Appl. Publ. Nos. 2017/0218093, 2018/0282385, 2018/0291311, or 2016/0122445, which are each incorporated herein by reference. For example, the GTF identified as GTF 8117 (SEQ ID NO:30), GTF 6831 (SEQ ID NO:32), or GTF 5604 (SEQ ID NO:33) in US2018/0282385 can be used, or the GTF identified as GTF 2919 (SEQ ID NO:5), GTF 2918 (SEQ ID NO:9), GTF 2920 (SEQ ID NO:13), or GTF 2921 (SEQ ID NO:17) in US2016/0122445 can be used, or a GTF comprising an amino acid sequence that is about 100% identical to, or at least about 90%, 91%, 92%, 93%, 94%, 95%, 96%, 97%, 98%, 98.5%, 99%, or 99.5% identical to, the amino acid sequence of any of these GTF enzymes (and having GTF activity) can be used.

A dextransucrase herein is capable of producing dextran comprising about, or at least about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% alpha-1,6-glycosidic linkages, for example. Such a percent alpha-1,6 linkage profile takes into account the total of all linkages in the dextran (main chains of alpha-1,6 glucan and, if present, branch portions therefrom). Dextran as disclosed elsewhere herein such as in a homopolymer or graft-copolymer can have any of the foregoing linkage profiles, for example.

A dextransucrase herein is capable of producing dextran having a weight-average molecular weight (Mw) of about, at least about, or less than about, 1000, 2500, 5000, 7500, 10000, 25000, 50000, 75000, 100000, 150000, 200000, 250000, 500000, 750000, 1000000, 1000-10000, 1000-100000, 1000-1000000, 10000-100000, 10000-1000000, or 100000-1000000 Daltons, for example. In some aspects, the Mw is about, at least about, or less than about, 1, 2, 3, 4, 5, 6, 7, 8, 9, 10, 20, 30, 40, 50, 60, 70, 80, 90, 100, 110, 120, 130, 140, 150, 160, 170, 180, 190, 200, 10-50, 10-70, 10-80, 10-100, 10-120, 10-130, 10-150, 10-200, 25-50, 25-70, 25-80, 25-100, 25-120, 25-130, 25-150, 25-200, 50-70, 50-80, 50-100, 50-120, 50-130, 50-150, 50-200, 70-80, 70-100, 70-120, 70-130, 70-150, 70-200, 80-100, 80-120, 80-130, 80-150, 80-200, 100-120, 100-130, 100-150, 100-200, 120-130, 120-150, 120-200, 130-150, or 130-200 million Daltons, for example. Dextran as disclosed elsewhere herein such as in a homopolymer or graft-copolymer can have any of the foregoing molecular weight profiles, for example.

In some aspects, a GTF enzyme that synthesizes alpha-1,3-glucan herein can comprise an amino acid sequence that is about 100% identical to, or at least about 90%, 91%, 92%, 93%, 94%, 95%, 96%, 97%, 98%, 98.5%, 99%, or 99.5% identical to, SEQ ID NO:2, 4, 6, 8, 10, 12, 14, 16, 18, 20, 26, 28, 30, 34, or 59, or amino acid residues 55-960 of SEQ ID NO:4, residues 54-957 of SEQ ID NO:65, residues 55-960 of SEQ ID NO:30, residues 55-960 of SEQ ID NO:28, or residues 55-960 of SEQ ID NO:20, and have GTF activity; these amino acid sequences are disclosed in U.S. Patent Appl. Publ. No. 2019/0078063, which is incorporated herein by reference. It is noted that such a GTF enzyme comprising SEQ ID NO:2, 4, 8, 10, 14, 20, 26, 28, 30, 34, or amino acid residues 55-960 of SEQ ID NO:4, residues 54-957 of SEQ ID NO:65, residues 55-960 of SEQ ID NO:30, residues 55-960 of SEQ ID NO:28, or residues 55-960 of SEQ ID NO:20, can synthesize alpha-glucan comprising at least about 90% (˜100%) alpha-1,3 linkages. A GTF enzyme that synthesizes alpha-1,3-glucan in some aspects can be that identified as GTF 0974 (SEQ ID NO:13 herein, SEQ ID NO:110 in US2018/0291311), or a GTF comprising an amino acid sequence that is about 100% identical to, or at least about 90%, 91%, 92%, 93%, 94%, 95%, 96%, 97%, 98%, 98.5%, 99%, or 99.5% identical to, the foregoing amino acid sequence of GTF 0974 (and having GTF activity). Any of the foregoing GTF enzyme amino acid sequences can be modified as described herein to increase product yield, modify product molecular weight, and/or enhance GTF performance and/or stability.

A GTF enzyme for producing alpha-1,3-glucan herein can, in some aspects, synthesize alpha-1,3-glucan at a yield of at least about 40%, 45%, 50%, 55%, 60%, 65%, 70%, 75%, 80%, 85%, 90%, 91%, 92%, 93%, 94%, 95%, or 96%. Yield in some aspects can be measured based on the glucosyl component of the reaction, and/or as measured using HPLC or NIR spectroscopy. Yield can be achieved in a reaction conducted for about 16-24 hours (e.g., ˜20 hours), for example. Examples of such a GTF enzyme are those having an amino acid sequence modified such that the enzyme produces more products (alpha-1,3-glucan and fructose), and less by-products (e.g., glucose, oligosaccharides such as leucrose), from a given amount of sucrose substrate. For example, one, two, three, four, or more amino acid residues of the catalytic domain of an alpha-1,3-glucan-producing GTF herein can be modified/substituted to obtain a GTF enzyme that produces more products. Examples of a suitable modified GTF enzyme are disclosed in Tables 3-7 of U.S. Patent Appl. Publ. No. 2019/0078063. A modified GTF enzyme, for example, can comprise one or more amino acid substitutions corresponding with those in Tables 3-7 (ibid.) that is/are associated with an alpha-1,3-glucan yield of at least 40% (the position numbering of such at least one substitution corresponds with the position numbering of SEQ ID NO:62 as disclosed in U.S. Patent Appl. Publ. No. 2019/0078063). A set of amino acid modifications as listed in Tables 6 or 7 (ibid.) can be used, for example.

The amino acid sequence of a GTF enzyme for alpha-1,3-glucan synthesis in some aspects has been modified such that the enzyme produces alpha-1,3-glucan with a molecular weight (DPw) that is lower than the molecular weight of alpha-1,3-glucan produced by its corresponding parent GTF. Examples of a suitable modified GTF enzyme are disclosed in Tables 3 and 4 of U.S. Patent Appl. Publ. No. 2019/0276806, which is incorporated herein by reference. A modified GTF enzyme, for example, can comprise one or more amino acid substitutions corresponding with those in Tables 3 and/or 4 (ibid.) that is/are associated with an alpha-1,3-glucan product molecular weight that is at least 5% less than the molecular weight of alpha-1,3-glucan produced by parent enzyme (the position numbering of such at least one substitution corresponds with the position numbering of SEQ ID NO:62 as disclosed in U.S. Patent Appl. Publ. No. 2019/0276806). A set of amino acid modifications as listed in Table 4 (ibid.) can be used, for example.

The amino acid sequence of a GTF enzyme for alpha-1,3-glucan synthesis in some aspects has been modified such that the enzyme produces alpha-1,3-glucan with a molecular weight (DPw) that is higher than the molecular weight of alpha-1,3-glucan produced by its corresponding parent GTF. Examples of a suitable modified GTF enzyme are disclosed in Tables 3, 4 and 5 of U.S. Patent Appl. Publ. No. 2019/0078062, which is incorporated herein by reference. A modified GTF enzyme, for example, can comprise one or more amino acid substitutions corresponding with those in Tables 3, 4 and/or 5 (ibid.) that is/are associated with an alpha-1,3-glucan product molecular weight that is at least 5% higher than the molecular weight of alpha-1,3-glucan produced by parent enzyme (the position numbering of such at least one substitution corresponds with the position numbering of SEQ ID NO:62 as disclosed in U.S. Patent Appl. Publ. No. 2019/0078062). A set of amino acid modifications as listed in Table 5 (ibid.) can be used, for example.

In some aspects, a modified GTF for alpha-1,3-glucan synthesis (i) comprises at least one amino acid substitution or a set of amino acid substitutions (as described above regarding yield or molecular weight), and (ii) comprises or consists of a GTF catalytic domain that is at least about 90%, 91%, 92%, 93%, 94%, 95%, 96%, 97%, 98%, 99%, or 99.5% identical to amino acid residues 55-960 of SEQ ID NO:4, amino acid residues 54-957 of SEQ ID NO:65, amino acid residues 55-960 of SEQ ID NO:30, amino acid residues 55-960 of SEQ ID NO:28, or amino acid residues 55-960 of SEQ ID NO:20 (each of these sequences as disclosed in U.S. Patent Appl. Publ. No. 2019/0078063, which is incorporated herein by reference). Each of these subsequences are the approximate catalytic domains of each respective reference sequence, and produce alpha-1,3-glucan comprising at least about 50% (e.g., ≥90% or ≥95%) alpha-1,3 linkages. In some aspects, a modified GTF (i) comprises at least one amino acid substitution or a set of amino acid substitutions (as described above), and (ii) comprises or consists of an amino acid sequence that is at least about 90%, 91%, 92%, 93%, 94%, 95%, 96%, 97%, 98%, 99%, or 99.5% identical to SEQ ID NO:62 or a subsequence thereof such as SEQ ID NO:4 (without start methionine thereof) or positions 55-960 of SEQ ID NO:4 (approximate catalytic domain) (each of these sequences as disclosed in U.S. Patent Appl. Publ. No. 2019/0078063).

In the present disclosure, SEQ ID NOs:5, 6, 7, 8, 9 and 10 (Table A) are the same amino acid sequences as, respectively, SEQ ID NOs:4, 65, 30, 28, 20 and 62 as disclosed in U.S. Patent Appl. Publ. No. 2019/0078063. Thus, each of presently disclosed SEQ ID NOs:5, 6, 7, 8, 9 and 10 can be used in any of the disclosed aspects, as appropriate. For example, a GTF enzyme that synthesizes alpha-1,3-glucan herein can comprise an amino acid sequence that is about 100% identical to, or at least about 90%, 91%, 92%, 93%, 94%, 95%, 96%, 97%, 98%, 98.5%, 99%, or 99.5% identical to, SEQ ID NO:5, 6, 7, 8, 9, or 10, or amino acid residues 55-960 of SEQ ID NO:5, residues 54-957 of SEQ ID NO:6, residues 55-960 of SEQ ID NO:7, residues 55-960 of SEQ ID NO:8, or residues 55-960 of SEQ ID NO:9. Any of these sequences can be modified as described herein to affect alpha-1,3-glucan yield and/or molecular weight and/or stability, for example.

In some aspects, a GTF enzyme for alpha-1,3-glucan synthesis has been modified such that the enzyme has enhanced performance and/or stability benefit(s). Such a modification can be, for example, by having one, two, three, four, five, six, seven, eight, nine, ten, or more amino acid substitutions as compared to a corresponding parent GTF enzyme (e.g., a wild type mature GTF or active subsequence thereof such as a catalytic domain). Exemplary performance and/or stability benefits herein include one or more of increased thermal stability, increased storage stability, increased solubility, better pH profile, increased specific activity, modified substrate specificity, modified substrate binding, modified pH-dependent activity, modified pH-dependent stability, increased oxidative stability, increased expression, and/or increased glucan product yield (and/or decreased byproduct [e.g., leucrose] yield). In some aspects, a performance benefit is realized at a relatively low temperature (e.g., <5° C.) or at a relatively high temperature (e.g., >40° C.). An increase in any of the foregoing features can be by about, or at least about, 5%, 10%, 15%, 20%, 25%, or 30%, for example, as compared to the respective activity of a parent GTF enzyme that has not been modified.

Some examples of modified alpha-1,3-glucan-producing GTF enzymes herein having enhanced performance and/or stability benefit(s) comprise or consist of SEQ ID NO:3 (vGTFJ) or 4. It is noted that SEQ ID NOs:3 and 4 are both derivable from SEQ ID NO:5 (GTF 6855), for example (e.g., SEQ ID NO:5 can be a backbone for making substitutions to render SEQ ID NOs:3 and 4).

It is noted that, as compared to SEQ ID NO:5, SEQ ID NO:3 has the following amino acid substitutions: Tyr-8-Asn (i.e., position 8 is substituted with Asn, in place of Tyr, as compared to SEQ ID NO:5), Val-9-Ala, Leu-336-Tyr, Gln-411-Leu, Phe-430-Tyr, Lys-448-Ala, Arg-564-Ser, Thr-1254-Gln and Glu-1273-Phe (aside from having a valine at position 1). The positions of each of these substitutions correspond, respectively, with positions Tyr-185, Val-186, Leu-513, Gln-588, Phe-607, Lys-625, Arg-741, Thr-1431 and Glu-1450 of SEQ ID NO:10, which is used as a reference sequence herein.

It is noted that, as compared to SEQ ID NO:5, SEQ ID NO:4 has the following amino acid substitutions: Leu-336-Tyr, Phe-430-Tyr, Ile-431-Val, Lys-448-Ala, Arg-564-Ser, Val-1011-Glu, Lys-1150-His, Glu-1155-Ala, Asp-1241-Lys, Ala-1242-Glu, Ser-1243-Gly, Thr-1244-Ser, Arg-1247-Leu and Leu-1248-Val (aside from having a valine at position 1). The positions of each of these substitutions correspond, respectively, with positions Leu-513, Phe-607, Ile-608, Lys-625, Arg-741, Val-1188, Lys-1327, Glu-1332, Asp-1418, Ala-1419, Ser-1420, Thr-1421, Arg-1424 and Leu-1425 of SEQ ID NO:10, which is used as a reference sequence herein.

In some aspects of the present disclosure, a modified GTF enzyme can comprise or consist of an amino acid sequence that is at least about 90%, 91%, 92%, 93%, 94%, 95%, 96%, 97%, 98%, 99%, or 99.5% identical to SEQ ID NO:3, and have one or more of (or all of) the following amino acid residues: 8-Asn, 9-Ala, 336-Tyr, 411-Leu, 430-Tyr, 448-Ala, 564-Ser, 1254-Gln, and/or 1273-Phe. The valine at position 1 of SEQ ID NO:3 in any of the foregoing aspects can optionally instead be a methionine, or can be deleted.

In some aspects of the present disclosure, a modified GTF enzyme can comprise or consist of an amino acid sequence that is at least about 90%, 91%, 92%, 93%, 94%, 95%, 96%, 97%, 98%, 99%, or 99.5% identical to SEQ ID NO:4, and have one or more of (or all of) the following amino acid residues: 336-Tyr, 430-Tyr, 431-Val, 448-Ala, 564-Ser, 1011-Glu, 1150-His, 1155-Ala, 1241-Lys, 1242-Glu, 1243-Gly, 1244-Ser, 1247-Leu, and/or 1248-Val. The valine at position 1 of SEQ ID NO:4 in any of the foregoing aspects can optionally instead be a methionine, or can be deleted.

Any of the amino acids listed above in aspects related to SEQ ID NOs:3 and 4 can optionally instead be another amino acid selected from Table B based on amino acid conservation.

TABLE B
Amino Acid Conservation

| For Amino Acid | Code | Replace with any of: |
|---|---|---|
| Alanine | A | D-Ala, Gly, beta-Ala, L-Cys, D-Cys |
| Arginine | R | D-Arg, Lys, D-Lys, homo-Arg, D-homo-Arg, Met, Ile, D- |
| | | Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S—Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, b-Ala, Acp |
| Isoleucine | | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, Met, D-Met, |
| | | Ile, D-Ile, Orn, D-Orn |
| Methionine | M | D-Met, S—Me-Cys, Ile, D-Ile, Leu, D-Leu, Val, D-Val |
| Phenylalanine | F | D-Phe, Tyr, D-Thr, L-Dopa, His, D-His, Trp, D-Trp, |
| | | Trans-3,4, or 5-phenylproline, cis-3,4, or 5- |
| | | phenylproline |
| Proline | P | D-Pro, L-I-thioazolidine-4- carboxylic acid, D-or L-1- |
| | | oxazolidine-4-carboxylic acid |
| Serine | S | D-Ser, Thr, D-Thr, allo-Thr, Met, D-Met, Met(O), D- |
| | | Met(O), L-Cys, D-Cys |
| Threonine | T | D-Thr, Ser, D-Ser, allo-Thr, Met, D-Met, Met(O), D- |
| | | Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

In some aspects, a modified alpha-1,3-glucan-producing GTF enzyme having enhanced performance and/or stability benefit(s) comprises one, two, three, four, five, six, seven, eight, nine, ten, or more amino acid substitution(s) at a position(s) corresponding with amino acid residue(s) Tyr-185, Val-186, Leu-513, Gln-588, Phe-607, Ile-608, Lys-625, Arg-741, Val-1188, Lys-1327, Glu-1332, Asp-1418, Ala-1419, Ser-1420, Thr-1421, Arg-1424, Leu-1425, Thr-1431, and/or Glu-1450 of SEQ ID NO:10. For example, a modified alpha-1,3-glucan-producing GTF enzyme can comprise amino acid substitutions at positions corresponding with the following amino acid residues of SEQ ID NO:10:
(i) Tyr-185, Val-186, Leu-513, Gln-588, Phe-607, Lys-625, Arg-741, Thr-1431 and/or Glu-1450 (these positions correspond to those in the GTF of SEQ ID NO:3);
(ii) Leu-513, Phe-607, Ile-608, Lys-625, Arg-741, Val-1188, Lys-1327, Glu-1332,
Asp-1418, Ala-1419, Ser-1420, Thr-1421, Arg-1424, and/or Leu-1425 (these positions correspond to those in the GTF of SEQ ID NO:4);
(iii) Tyr-185, Val-186, Lys-625, Thr-1431, and/or Glu-1450,
(iv) Ile-608, Lys-625, Lys-1327, Glu-1332, Asp-1418, Ala-1419, Ser-1420, Thr-1421, Arg-1424, and/or Leu-1425,
(v) Leu-513, Gln-588, Phe-607, Lys-625, and/or Arg-741,
(vi) Leu-513, Phe-607, Ile-608, Lys-625, and/or Arg-741, and/or
(vii) Leu-513, Phe-607, Lys-625, and/or Arg-741.

In some aspects regarding a modified alpha-1,3-glucan-producing GTF enzyme having enhanced performance and/or stability benefit(s),
(a) the amino acid substitution at a position corresponding with amino acid residue Tyr-185 of SEQ ID NO:10 can be with an Asn residue, or any residue that is conserved with Asn (e.g., Table B);
(b) the amino acid substitution at a position corresponding with amino acid residue Val-186 of SEQ ID NO:10 can be with an Ala residue, or any residue that is conserved with Ala (e.g., Table B);
(c) the amino acid substitution at a position corresponding with amino acid residue Leu-513 of SEQ ID NO:10 can be with a Tyr, Phe, or Trp residue, or any residue that is conserved with Tyr, Phe, or Trp (e.g., Table B);
(d) the amino acid substitution at a position corresponding with amino acid residue Gln-588 of SEQ ID NO:10 can be with a Leu residue, or any residue that is conserved with Leu (e.g., Table B);
(e) the amino acid substitution at a position corresponding with amino acid residue Ile-608 of SEQ ID NO:10 can be with a Val or Tyr residue, or any residue that is conserved with Val or Tyr (e.g., Table B);
(f) the amino acid substitution at a position corresponding with amino acid residue Lys-625 of SEQ ID NO:10 can be with an Ala residue, or any residue that is conserved with Ala (e.g., Table B);
(g) the amino acid substitution at a position corresponding with amino acid residue Arg-741 of SEQ ID NO:10 can be with a Ser residue, or any residue that is conserved with Ser (e.g., Table B);
(h) the amino acid substitution at a position corresponding with amino acid residue Val-1188 of SEQ ID NO:10 can be with a Glu residue, or any residue that is conserved with Glu (e.g., Table B);
(i) the amino acid substitution at a position corresponding with amino acid residue Lys-1327 of SEQ ID NO:10 can be with a His residue, or any residue that is conserved with His (e.g., Table B);
(j) the amino acid substitution at a position corresponding with amino acid residue Glu-1332 of SEQ ID NO:10 can be with an Ala residue, or any residue that is conserved with Ala (e.g., Table B);
(k) the amino acid substitution at a position corresponding with amino acid residue Asp-1418 of SEQ ID NO:10 can be with a Lys residue, or any residue that is conserved with Lys (e.g., Table B);
(l) the amino acid substitution at a position corresponding with amino acid residue Ala-1419 of SEQ ID NO:10 can be with a Glu residue, or any residue that is conserved with Glu (e.g., Table B);
(m) the amino acid substitution at a position corresponding with amino acid residue Ser-1420 of SEQ ID NO:10 can be with a Gly residue, or any residue that is conserved with Gly (e.g., Table B);
(n) the amino acid substitution at a position corresponding with amino acid residue Thr-1421 of SEQ ID NO:10 can be with a Ser residue, or any residue that is conserved with Ser (e.g., Table B);
(o) the amino acid substitution at a position corresponding with amino acid residue Arg-1424 of SEQ ID NO:10 can be with a Leu residue, or any residue that is conserved with Leu (e.g., Table B);
(p) the amino acid substitution at a position corresponding with amino acid residue Leu-1425 of SEQ ID NO:10 can be with a Val residue, or any residue that is conserved with Val (e.g., Table B);
(q) the amino acid substitution at a position corresponding with amino acid residue Thr-1431 of SEQ ID NO:10 can be with a Gln residue, or any residue that is conserved with Gln (e.g., Table B); and/or
(r) the amino acid substitution at a position corresponding with amino acid residue Glu-1450 of SEQ ID NO:10 can be with a Phe residue, or any residue that is conserved with Phe (e.g., Table B).

Although it is believed that a modified alpha-1,3-glucan-producing GTF enzyme in some aspects need only have a catalytic domain, the modified GTF can be comprised within a larger amino acid sequence. For example, a catalytic domain may be linked at its C-terminus to a glucan-binding domain, and/or linked at its N-terminus to a variable domain and/or signal peptide.

Although amino acid substitutions in a modified alpha-1,3-glucan-producing GTF enzyme are generally disclosed in some aspects with respect to corresponding positions in SEQ ID NO:10, such substitutions can alternatively be stated simply with respect to its/their position number in the amino acid sequence used to produce the modified GTF itself (e.g., SEQ ID NO:5 [optionally without start methionine thereof] or positions 55-960 of SEQ ID NO:5 [approximate catalytic domain]), as convenience may dictate. Such can be done simply by aligning the amino acid sequence with SEQ ID NO:10 and identifying the position number(s) of interest in the amino acid sequence based on its/their direct alignment with the corresponding position(s) in SEQ ID NO:10.

An alpha-1,3-glucan-producing GTF herein is capable of producing alpha-1,3-glucan comprising about, or at least about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% alpha-1,3-glycosidic linkages, for example. Alpha-1,3-glucan as disclosed elsewhere herein such as in a homopolymer or graft-copolymer can have any of the foregoing linkage profiles, for example.

An alpha-1,3-glucan-producing GTF herein is capable of producing alpha-1,3-glucan with a DPw, DPn, or DP of about, or at least about, 11, 12, 15, 20, 25, 30, 35, 40, 45, 50, 55, 60, 65, 70, 75, 80, 85, 90, 95, 100, 150, 200, 300, 400, 500, 600, 700, 800, 900, 1000, 1100, 1200, 1300, 1400, 1500, 1600, or 1650, for example. DPw, DPn, or DP can optionally be expressed as a range between any two of these values. Merely as examples, the DPw, DPn, or DP can be about 100-1650, 200-1650, 300-1650, 400-1650, 500-1650, 600-1650, 700-1650, 100-1250, 200-1250, 300-1250, 400-1250, 500-1250, 600-1250, 700-1250, 100-1000, 200-1000, 300-1000, 400-1000, 500-1000, 600-1000, 700-1000, 100-900, 200-900, 300-900, 400-900, 500-900, 600-900, 700-900, 11-25, 12-25, 11-22, 12-22, 11-20, 12-20, 20-300, 20-200, 20-150, 20-100, 20-75, 30-300, 30-200, 30-150, 30-100, 30-75, 50-300, 50-200, 50-150, 50-100, 50-75, 75-300, 75-200, 75-150, 75-100, 100-300, 100-200, 100-150, 150-300, 150-200, or 200-300. Alpha-1,3-glucan as disclosed elsewhere herein such as in a homopolymer or graft-copolymer can have any of the foregoing molecular weight profiles, for example.

In some aspects, a GTF enzyme can be any as disclosed herein and include 1-300 (or any integer there between [e.g., 10, 15, 20, 25, 30, 35, 40, 45, or 50]) residues on the N-terminus and/or C-terminus. Such additional residues can be from a corresponding wild type sequence from which the GTF enzyme is derivable, or can be a heterologous sequence such as an epitope tag (at either N- or C-terminus) or a heterologous signal peptide (at N-terminus), for example. A GTF enzyme herein typically lacks an N-terminal signal peptide; such an enzyme can optionally be characterized as being mature if its signal peptide was removed during a secretion process.

A GTF enzyme herein can typically be derived from bacteria. Examples of bacterial GTF enzymes are those derived from a Streptococcus species, Leuconostoc species, or Lactobacillus species. Examples of Streptococcus species include S. salivarius, S. sobrinus, S. dentirousetti, S. downei, S. mutans, S. oralis, S. gallolyticus and S. sanguinis. Examples of Leuconostoc species include L. mesenteroides, L. amelibiosum, L. argentinum, L. carnosum, L. citreum, L. cremoris, L. dextranicum and L. fructosum. Examples of Lactobacillus species include L. acidophilus, L. delbrueckii, L. helveticus, L. salivarius, L. casei, L. curvatus, L. plantarum, L. sakei, L. brevis, L. buchneri, L. fermentum and L. reuteri.

A GTF enzyme herein can be prepared by fermentation of an appropriately engineered microbial strain, for example. Recombinant enzyme production by fermentation can be done, for example, using microbial species such as E. coli, Bacillus strains (e.g., B. subtilis), Ralstonia eutropha, Pseudomonas fluorescens, Saccharomyces cerevisiae, Pichia pastoris, Hansenula polymorpha, and species of Aspergillus (e.g., A. awamori) and Trichoderma (e.g., T. reesei) (e.g., see Adrio and Demain, Biomolecules 4:117-139, 2014, which is incorporated herein by reference). A nucleotide sequence encoding a GTF amino acid sequence is typically linked to a heterologous promoter sequence to create an expression cassette for the enzyme, and/or is codon-optimized accordingly. Such an expression cassette can be incorporated in a suitable plasmid or integrated into the microbial host chromosome. The expression cassette can include a transcriptional terminator nucleotide sequence following the amino acid coding sequence. The expression cassette can also include, between the promoter sequence and GTF amino acid coding sequence, a nucleotide sequence encoding a signal peptide (e.g., heterologous signal peptide) that is designed for direct secretion of the GTF enzyme. At the end of fermentation, cells can be ruptured accordingly (generally when a signal peptide for secretion is not employed) and the GTF enzyme can be isolated using methods such as precipitation, filtration, and/or concentration. Alternatively, a lysate or extract comprising a GTF can be used without further isolation. If the GTF was secreted (i.e., it is present in the fermentation broth), it can optionally be used as isolated from, or as comprised in, the fermentation broth. The activity of a GTF enzyme can be confirmed by biochemical assay, such as measuring its conversion of sucrose to glucan polymer.

Alpha-glucan produced in step (b) of producing a food product/precursor in some aspects comprises a graft copolymer comprising:
(i) an alpha-1,6-glucan (dextran) backbone, wherein at least about 50% of the glycosidic linkages of the alpha-1,6-glucan (dextran) backbone are alpha-1,6 linkages, and
(ii) at least one alpha-1,3-glucan side chain, wherein at least about 50% of the glycosidic linkages of the alpha-1,3-glucan chain are alpha-1,3 linkages.

Such a graft copolymer can be aqueous-soluble or aqueous-insoluble. Dextran backbone of an alpha-glucan graft copolymer herein can be dextran as presently disclosed, for example, or can be as disclosed (e.g., molecular weight, linkage/branching profile, production method) in U.S. Patent Appl. Publ. Nos. 2016/0122445, 2017/0218093, 2018/0282385, 2020/0165360, or 2019/0185893, which are each incorporated herein by reference. In some aspects, a dextran backbone (before being integrated into a graft copolymer) has been alpha-1,2- and/or alpha-1,3-branched, the percent alpha-1,2 and/or alpha-1,3 branching of a backbone of a graft copolymer herein can be about, at least about, or less than about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 2-25%, 2-20%, 2-15%, 2-10%, 5-25%, 5-20%, 5-15%, 5-10%, 7-13%, 8-12%, 9-11%, 10-25%, 10-20%, or 10-15%, for example. Alpha-1,3-glucan side chain(s) of an alpha-glucan graft copolymer herein can be alpha-1,3-glucan as presently disclosed, for example, or can be as disclosed (e.g., molecular weight, linkage profile), in U.S. Pat. Nos. 7,000,000, 8,871,474, 10301604, or 10260053, or U.S. Patent Appl. Publ. Nos. 2019/0112456, 2019/0078062, 2019/0078063, 2018/0340199, 2018/0021238, 2018/0273731, 2017/0002335, 2015/0232819, 2015/0064748, 2020/0165360, 2020/0131281, or 2019/0185893, which are each incorporated herein by reference.

One, two, three, or more different GTF enzymes that synthesize alpha-1,6-glucan herein can be used, for example, in step (b) of producing a food product/precursor. Similarly, one, two, three, or more different GTF enzymes that synthesize alpha-1,3-glucan herein can be used, for example. In some aspects, an alpha-1,6-glucan-producing GTF(s) can be added to (made to contact) a food product/precursor before adding an alpha-1,3-glucan-producing GTF(s), while in some aspects both these types of GTF enzymes can be added at about the same time (simultaneously). Still, in some aspects, an alpha-1,3-glucan-producing GTF(s) can be added to a food product/precursor before adding an alpha-1,6-glucan-producing GTF(s). Still, in some aspects, a dextran as disclosed herein, but produced exogenously to the food product/precursor, can be added as an ingredient to a food product/precursor to which an alpha-1,3-glucan-producing GTF has already been added or will be added. While not being held to any particular theory, it is believed that addition of at least one alpha-1,6-glucan-producing GTF (and/or exogenously produced dextran) and at least one alpha-1,3-glucan-producing GTF in step (b) of producing a food product/precursor allows for production of a dextran-alpha-1,3-glucan graft copolymer as presently disclosed, possibly along with production of dextran and/or alpha-1,3-glucan homopolymer(s) (i.e., alpha-1,6-glucan and/or alpha-1,3-glucan produced independent from the production of graft copolymer). However, it is believed possible that, in some aspects, only dextran and/or alpha-1,3-glucan homopolymer(s) is/are produced with little (e.g., <5 wt % of all glucan products) or no production of graft copolymer.

The molecular weight and/or linkage profile of alpha-glucan produced by a GTF enzyme (dextransucrase or alpha-1,3-glucan-producing GTF) as generally disclosed above can be as observed, for example, in an isolated reaction consisting of, or essentially of, water, sucrose, GTF enzyme and optionally one or more salts and/or buffer. In some aspects, the molecular weight and/or linkage profile of alpha-glucan as produced by one or both of these types of GTF enzyme in a food product/precursor herein may be different from what is produced in the foregoing isolated reaction.

In some aspects, the ratio of a GTF enzyme that synthesizes alpha-1,6-glucan to a GTF enzyme that synthesizes alpha-1,3-glucan in step (b) is about 85:15 to about 95:5. Yet, in some aspects, an alpha-1,6-glucan-producing GTF to alpha-1,3-glucan-producing GTF ratio can be about 97.5:2.5, 95:5, 92.5:7.5, 91:9, 90:10, 89:11, 87.5:12.5, 85:15, 82.5:17.5, 80:20, 70:30, 60:40, 50:50, 40:60, 30:70, 20:80, 10:90, 5:95, or 2.5:97.5, or range between any two of these ratios (e.g., about 82.5:17.5 to 97.5:2.5, 87.5:12.5 to 92.5:7.5, 89:11 to 91:9, 17.5:82.5 to 2.5:97.5, 12.5:87.5 to 7.5:92.5, 11:89 to 9:91). The amount of each enzyme (active enzyme) for purposes of determining a ratio thereof herein can be on a molar, weight, or GTF activity basis, for example. The activity of a GTF enzyme for preparing a ratio herein can optionally be determined as disclosed in U.S. Patent Appl. Publ. No. 2014/0087431, which is incorporated herein by reference, and/or as disclosed in the below Examples. For example, a full (e.g., “100%”) complement of a GTF enzyme for setting up a ratio herein can be that amount of enzyme that can convert most of (e.g., >95%, >98%, >99%), or all of, sucrose in a GTF reaction comprising or consisting of water, sucrose (e.g., 50 or 100 g/L), the GTF, and optionally buffer/salt in a given amount of time (e.g., 6, 12, 18, 24, 30, or 36 hours); such a measured amount can optionally be characterized as a normalized amount of GTF.

A GTF enzyme (or any other enzyme as presently disclosed) for use in a method herein is typically in purified form. A purified enzyme can be essentially free from insoluble and/or soluble components of an organism/cell used to produce the enzyme, and/or any medium that was used for cellular fermentation of the enzyme. In some aspects, a purified enzyme denotes an enzyme preparation that contains less than 10%, 8%, 6%, 5%, 4%, 3%, 2%, 1%, 0.5%, or 0.1% by weight of other material (e.g., polypeptide material) with which the enzyme is natively or recombinantly associated. In some aspects, a GTF and/or any other enzyme herein is not comprised in or otherwise associated with (e.g., expressed by) a microbial (e.g., bacterial, yeast, fungal, algal) cell that might be present (e.g., endogenously or purposely added) in a food product/precursor herein; however, in some aspects a GTF and/or any other enzyme herein is comprised in or otherwise associated with (e.g., expressed by) a microbial (e.g., bacterial, yeast, fungal, algal) cell such as one that heterologously expresses the enzyme(s) (i.e., recombinant cells). Contacting a food product/precursor with a GTF enzyme(s) herein typically is not performed in an oral cavity or other environment in which unpurified/non-isolated GTF enzymes can possibly be present.

A GTF enzyme (or any other enzyme as presently disclosed) for use in a method herein can be comprised in a sterile-filtered preparation, for example. In some aspects, an enzyme can be sterile-filtered inline while applying the enzyme to a food product/precursor during step (b) herein. In some aspects, an enzyme can be added to a food product/precursor that has been pasteurized (after pasteurization), or alternatively an enzyme can be added before pasteurizing the food product/precursor. In some aspects, an enzyme can be added to a food product/precursor that has been fermented (after fermentation), or alternatively an enzyme can be added during or before fermenting the food product/precursor. A GTF enzyme (or any other enzyme as presently disclosed) in some aspects for use in a method herein can be comprised in a preparation (for adding in step [b] herein) that is substantially free of (e.g., <0.5, <0.1, <0.05 wt %) any other enzyme(s) such as a lipase, protease, amylase, mannanase, pectinase, cellulase, and/or p-nitrobenzylesterase; such a preparation typically has little or no detectable activity(ies) of such other enzyme(s).

A food product/precursor herein can be brought into contact with one or more GTF enzymes in step (b) by mixing/stirring/blending, for example. Incubation of GTF enzyme(s) in the food product/precursor can be for a time sufficient for the GTF(s) to produce alpha-glucan in the food product/precursor, such as for about, or at least about, 0.5, 1, 2, 3, 4, 5, 6, 7, 8, 9, 10, 12, 15, 18, 24, 30, 36, 42, 48, 72, or 96 hours, or for about, or at least about, 5, 10, 15, 20, 25, 30, 35, 40, 45, 50, 55, or 60 days (or a range between any two of these hours and/or days). The temperature for incubating one or more GTF enzymes in a food product/precursor herein can be about 2, 3, 4, 5, 6, 7, 8, 9, 10, 12, 14, 16, 18, 20, 22, 24, 25, 26, 28, 30, 32, 34, 36, 38, 40, 42, 44, 46, 48, 50, 2-5, 2-10, 2-15, 2-20, 2-25, 2-30, 2-35, 2-40, 2-45, 2-50, 3-5, 3-10, 3-15, 3-20, 3-25, 3-30, 3-35, 3-40, 3-45, 3-50, 5-10, 5-15, 5-20, 5-25, 5-30, 5-35, 5-40, 5-45, 5-50, 15-20, 15-25, 15-30, 15-35, 15-40, 15-45, 15-50, 20-25, 20-30, 20-35, 20-40, 20-45, 20-50, 25-30, 25-35, 25-40, 25-45, 25-50, 30-35, 30-40, 30-45, or 30-50° C., for example.

Typically, a food product/precursor brought into contact with a GTF enzyme herein contains water (i.e., it is an aqueous composition), and/or water is introduced to the food product/precursor before or during contacting with GTF enzyme. GTF enzyme can be added to a food product/precursor in dry form (e.g., powder, flakes, lyophilized enzyme preparation) (typically to an aqueous food product/precursor) or wet form. In some aspects, a food product/precursor can be combined with a GTF enzyme under dry conditions (resulting combination is dry), after which time water or an aqueous solution is added, which in turn allows GTF production of alpha-glucan to proceed. The water content of a food product/precursor as provided in step (a), or in step (b) following addition of GTF enzyme, can be about, or at least about, 10, 20, 30, 40, 50, 60, 70, 80, 90, 95, or 99 wt %, for example. The pH of a food product/precursor herein, and/or the pH for incubating one or more GTF enzymes in a food product/precursor herein, can be about 3.0, 3.5, 4.0, 4.5, 5.0, 5.5, 6.0, 6.5, 7.0, 7.5, 8.0, 8.5, 9.0, 9.5, 10.0, 10.5, 4.0-10.0, 4.0-9.0, 4.0-8.0, 4.5-10.0, 4.5-9.0, 4.5-8.0, 5.0-10.0, 5.0-9.0, 5.0-8.0, 5.5-10.0, 5.5-9.0, 5.5-8.0, 6.0-10.0, 6.0-9.0, or 6.0-8.0, for example. A food product/precursor in some aspects can be acidic (e.g., pH 3.0, 3.2, 3.5, 4.0, 4.5, 5.0, 5.5, 6.0, or 6.5), neutral (e.g., pH 6.5-7.5), or basic/alkaline (e.g., pH>7.5, 8.0, 8.5, 9.0, 9.5).

A GTF enzyme herein can optionally be provided in step (b) of a method by introducing a recombinantly engineered cell (e.g., a microbial cell such as a bacterial or fungal/yeast cell) to the food product/precursor provided in step (a), wherein the cell recombinantly (heterologously) expresses and secretes the GTF enzyme in and/or around the food product/precursor. Such a cell can be that of a microbe that is amenable to recombinant engineering and useful in food processing (e.g., fermentation), such as a microbial cell disclosed herein (as applicable). In some aspects, a recombinantly engineered cell that is provided to the food product/precursor in step (a) can be inactive and/or non-viable in some manner, such as by having been killed (but preferably in a manner that otherwise retains cellular shape/structure). For example, a cell can be rendered inactive and/or non-viable by being irradiated or being treated with a sterilizing agent/chemical (e.g., ethylene oxide). Typically, the means for cell inactivation and/or killing preserves at least some of the three-dimensional shape/structure of the cell, and/or ensures that a GTF enzyme(s) that had been expressed by the cell remains active and typically remains associated with the inactive/non-viable cell (e.g., such as by being associated with a cellular membrane via an optional transmembrane domain or membrane-binding domain of the GTF enzyme [e.g., fused to the GTF]). An inactive/non-viable cell typically is porous, and optionally can be immobilized on a support (e.g., an inert, water-insoluble material, such as of a particle or surface).

In some aspects, a food product/precursor herein can be brought into contact with one or more GTF enzymes by virtue of adding the food product/precursor to an aqueous composition comprising at least sucrose and the one or more GTF enzymes. While the food product/precursor in this aspect has at least some sucrose and water, a food product/precursor in some other aspects does not comprise sucrose and/or water. Such a GTF/sucrose-containing aqueous composition can optionally be referred to herein as a “GTF/sucrose starter composition”. Typically, one or more food precursors as presently disclosed (e.g., ingredients such as a liquid food product/precursor, beverage, RTD, fruit/vegetable purée, syrup, or juice or juice concentrate) can be added to a GTF/sucrose starter composition, although one or more food products themselves as presently disclosed (e.g., fruit or vegetable matter such as pieces [e.g., slices, cubes, or other shaped pieces]) can be added (typically in conjunction with adding a food precursor). In some aspects, a GTF/sucrose starter composition can already comprise at least one food product/precursor, such as any disclosed herein. The initial sucrose concentration of a GTF/sucrose starter composition can be as presently disclosed, for example, such as 5-60%, 5-50%, 5-40%, 10-60%, 10-50%, 10-40%, 20-60%, 20-50%, 20-40%, 30-60%, 30-50%, 30-40%, 40-60%, or 40-50% by weight. In some aspects, a GTF/sucrose starter composition has few (e.g., less than about 1, 0.5, 0.1, 0.05, or 0.01 wt %) or no saccharide compounds (e.g., one or more monosaccharides, disaccharides, oligosaccharides and/or polysaccharides, such as presently disclosed) aside from sucrose. A GTF/sucrose starter composition can comprise at least one alpha-1,6-glucan-producing GTF and/or an alpha-1,3-glucan-producing GTF as presently disclosed, for example. The temperature, pH and/or any other condition/parameter of this methodology (before and/or after adding one or more food products/precursors to a GTF/sucrose starter composition) can be as disclosed herein, for example. In some aspects, a GTF/sucrose starter composition can be incubated for about, at least about, or up to about, 10, 15, 20, 25, 30, 35, 40, 45, 50, 55, 60, 120, 180, 240, 300, 360, 420, 480, 540, 10-45, 10-40, 10-35, 10-30, 10-25, 10-20, 10-15, 15-45, 15-40, 15-35, 15-30, 15-25, 15-20, 20-45, 20-40, 20-35, 20-30, 20-25, 25-45, 25-40, 25-35, or 25-30 minutes, for example, before adding one or more food products/precursors. Any of these foregoing time periods can also apply to the period of time allowed to proceed after adding the food product/precursor to the GTF/sucrose starter composition, until optionally terminating the GTF activity (e.g., heat-inactivation at 90-100° C.) of the final food product/precursor. Typically, the one or more food products/precursors added to the GTF/sucrose starter composition comprises one or more saccharide compounds (e.g., one or more monosaccharides, disaccharides, oligosaccharides and/or polysaccharides, such as presently disclosed). In some aspects, adding one or more food product/precursors to a GTF/sucrose starter composition can be done to control or adjust the degree of thickening and/or texturization desired in the final food product/precursor being produced. The thickening and/or texturization that can be achieved by such methodology can be greater (e.g., about, or at least about, 10%, 25%, 50%, 75%, 100%, 150%, 200%, 250%, 300%, 400%, or 500% greater) than the thickening and/or texturization that would have been achieved if all the ingredients used to make the final food product/precursor had all been combined at about the same time. Merely as examples, the foregoing process of adding one or more food products/precursors to a GTF/sucrose starter composition can be used herein to produce a marmalade, gel/gelatin, pudding, custard, yogurt, or cream, optionally with one or more suspended solid food ingredients such as fruit or vegetable pieces.

A “food product/precursor” (i.e., a food product or precursor) as provided in step (a) of a method in some aspects of the present disclosure method can comprise sucrose that is endogenous to the food product/precursor (e.g., its sucrose is native), and/or can comprise sucrose that has been added to the food product/precursor (either during or after its preparation as an ingredient). Step (a) can thus optionally comprise adding sucrose to the food product/precursor. The sucrose content of a food product/precursor finally provided in step (a) herein, regardless of the original source of the sucrose, can be about, at least about, or less than about, 0.1, 0.5, 1, 2.5, 5, 7.7, 10, 15, 20, 25, 30, 40, 50, 60, or 70 wt %, for example. In some aspects, sucrose can be provided as white refined sucrose, or in an unrefined form such as disclosed in U.S. Pat. No. 9,719,121, for example, which is incorporated herein by reference. Sucrose can optionally be added to a food product/precursor when adding GTF enzyme to the food product/precursor.

In some aspects, a food product/precursor as provided in step (a) of a method herein further comprises at least one disaccharide in addition to sucrose, and/or at least one oligosaccharide. An oligosaccharide can have 3-15 or 3-20 monomeric units (i.e., DP3-DP15 or DP3-DP20), for example (e.g., DP3-DP5, DP3-DP6); thus, in some aspects, a polysaccharide herein has more than 15 or 20 monomeric units. A disaccharide and/or oligosaccharide herein can comprise only glucose monomeric units, for example, and/or one or more other types of monosaccharides (e.g., galactose, fructose, mannose) as monomeric units. Examples of disaccharides herein (in addition to sucrose) include maltose, isomaltose, lactose, lactosucrose, nigerose, leucrose, trehalulose, maltulose, isomaltulose, and turanose. Examples of oligosaccharides herein include gluco-oligosaccharides (gluco-oligomers) such as malto-oligosaccharides (MOS) and isomalto-oligosaccharides (IMO), and galacto-oligosaccharides (GOS).

A disaccharide and/or oligosaccharide can be added to a food product/precursor either during or after preparation of the food product/precursor. Such addition can be from a source physically outside of the food product/precursor (i.e., as an ingredient), and/or can be via in situ production in the food/precursor such as by one or more enzymes that are endogenous and/or exogenous to the food/precursor. An enzyme that is added to a food product/precursor (i.e., exogenous enzyme) for producing a disaccharide and/or oligosaccharide can be added, for example, in the same or similar manner in which a GTF enzyme herein is added (e.g., time, temperature, pH), and can be added before, during, or after the addition of GTF enzyme. Such an enzyme can be a transglucosidase (EC [enzyme code] 2.4.1.24) or a transgalactosylating beta-galactosidase. Suitable transglucosidases herein include FoodPro® TGO and those disclosed in U.S. Patent Appl. Publ. Nos. 2008/0229514 or 2015/0240279, or U.S. Pat. No. 4,689,296, all of which are incorporated herein by reference. An EC 2.4.1.24 transglucosidase (also termed as “1,4-alpha-glucan 6-alpha-glucosyltransferase”) can transfer an alpha-D-glucosyl residue of an alpha-1,4-glucan, -oligosaccharide (i.e., MOS), or -disaccharide (i.e., maltose) to the primary hydroxy group of free glucose or glucose in an alpha-1,4-glucan, -oligosaccharide (i.e., MOS), or -disaccharide. Thus, an EC 2.4.1.24 transglucosidase produces isomalto-oligosaccharides (IMO) (e.g., DP3-DP5 or DP3-DP6) in some aspects. Suitable transgalactosylating beta-galactosidases herein are disclosed in U.S. Patent Appl. Publ. No. 2013/0189746 or U.S. Pat. Nos. 10,531,672 or 10,683,523, for example, which are incorporated herein by reference. A transgalactosylating beta-galactosidase is an enzyme that degrades lactose by transferring the galactose of lactose to galactose, glucose, or other acceptor thereby producing galacto-oligosaccharides (GOS) (e.g., GOS can also be an acceptor for forming a longer GOS). A particular example of such an enzyme is Nurica™ (IFF). A transglucosidase or transgalactosylating beta-galactosidase herein can be dosed into a food product/precursor herein at about 0.1-1.5, 0.1-1.25, 0.1-1.0, 0.1-0.75, 0.1-0.5, 0.2-1.5, 0.2-1.25, 0.2-1.0, 0.2-0.75, 0.2-0.5, 0.5-1.5, 0.5-1.25, 0.5-1.0, 0.5-0.75, 0.75-1.5, 0.75-1.25, or 0.75-1.0% (v/w), for example.

In some aspects, a food product/precursor as provided in step (a) of a method herein has, aside from the sucrose, little (e.g., less than 0.5, 0.25, 0.1, 0.05, 0.025, or 0.01 wt %, or not detectable) or no disaccharides and/or oligosaccharides (or little or no particular disaccharide or oligosaccharide). A food product/precursor as produced in step (b) of a method herein can likewise have, for example, little of no disaccharides and/or oligosaccharides (or little or no particular disaccharide or oligosaccharide), and also have little (e.g., as above) or no sucrose. A disaccharide or oligosaccharide in such aspects can be any as disclosed herein (e.g., lactose, maltose, isomaltose, MOS, IMO, GOS). One or more glycosidase enzymes (glycosidic-active enzyme) can be used, for example, in a food product/precursor to reduce or eliminate the presence of disaccharide(s) and/or oligosaccharide(s), and can be added, for example, in the same or similar manner in which a GTF enzyme herein is added (e.g., time, temperature, pH), and can be added before, during, or after the addition of GTF enzyme. A glycosidase herein can be, for example, a beta-galactosidase (EC 3.2.1.23; e.g., lactase [EC 3.2.1.108]) or alpha-glucosidase (EC 3.2.1.20). Suitable beta-galactosidases herein include Bonlacta™ lactase (IFF) and lactases disclosed in U.S. patent Ser. No. 10/531,672, which is incorporated herein by reference. A lactase herein is a type of beta-galactosidase enzyme that catalyzes hydrolysis of lactose to glucose and galactose. Suitable alpha-glucosidases herein include those disclosed in U.S. Patent Appl. Publ. No. 2015/0240278, which is incorporated herein by reference. In some aspects, an alpha-glucosidase that is used in the disclosed method is able to hydrolyze an alpha-1,4 or alpha-1,6 glucosidic linkage, or and/or is unable to hydrolyze an alpha-1,3 glucosidic linkage. A glycosidase herein can be dosed into a food product/precursor herein at about 0.1-1.5, 0.1-1.25, 0.1-1.0, 0.1-0.75, 0.1-0.5, 0.2-1.5, 0.2-1.25, 0.2-1.0, 0.2-0.75, 0.2-0.5, 0.5-1.5, 0.5-1.25, 0.5-1.0, 0.5-0.75, 0.75-1.5, 0.75-1.25, or 0.75-1.0% (v/w), for example.

A food product/precursor in some aspects can be a dairy product/precursor, such as a dairy beverage or food. Suitable examples of a dairy product/precursor herein include milk, cheese, yogurt, dessert, cream, and butter. Milk herein can be whole milk (e.g., ˜3% fat), ˜2% fat milk, ˜1% fat milk (“low-fat”), or fat-free (non-fat) milk, for example. Milk, whether used directly as a beverage or as a precursor for preparing a dairy product/precursor herein, can be from a cow, goat, sheep, buffalo, yak, llama, camel, or horse, for example. Milk can optionally be pasteurized before, or after, contacting it with one or more GTF enzymes herein. A cheese herein can be, for example, hard or semi-hard cheese (e.g., cheddar, mozzarella, Swiss, parmesan, provolone), soft or semi-soft cheese (e.g., ricotta, cottage cheese, feta, American, brie), processed, or non-processed. A yogurt herein can be, for example, whole milk yogurt, low-fat yogurt, fat-free yogurt, or Greek yogurt (e.g., plain, low-fat, non-fat). A yogurt herein can optionally contain fruit and/or be flavored. A yogurt herein can optionally be drinkable (i.e., yogurt beverage). A dairy dessert in some aspects can be a pudding (e.g., whole milk, 2% milk), frozen yogurt (e.g., low-fat), ice cream (e.g., low-fat), sherbet, milk shake, gelato, or custard; thus, in some aspects a dairy dessert can be a frozen dairy dessert (e.g., ice cream, sherbet, milk shake, frozen yogurt, gelato). Ice cream can be hard ice cream or soft (soft-serve) ice cream, for example. A dairy beverage in some aspects can be milk, chocolate milk, coffee milk, flavored milk, yogurt beverage, kumis, ryazhenka, ayran, lassi, cholado, licuado, or kefir. A dairy cream can be clotted cream (e.g., ≥55% milkfat), heavy cream (e.g., ≥36% milkfat), whipping cream (e.g., 30%-36% milkfat), light cream (e.g., 18%-30% milkfat), sour cream (≥18% milkfat), half-and-half (e.g., 10.5%-18% milkfat), or ice cream (e.g., ≥10% milkfat). A dairy product/precursor herein can be lactose-free or have a reduced lactose content, for example. A dairy product/precursor herein can be a fermented dairy product/precursor (e.g., yogurt, buttermilk, creme fraiche, quark, fromage frais, soured milk, vinegar), for example. Some dairy products/precursors herein include dairy confections such as milk chocolate, white chocolate, caramel, and toffee. A dairy product/precursor in some aspects can be any as disclosed in Int. Patent Appl. Publ. No. WO2020/010176, or U.S. Patent Appl. Publ. Nos. 2013/0230623, 2005/0244541, 2017/0135360, 2009/0304864, 2017/0094987, 2003/0152685, or 2021/0282422, or U.S. Pat. Nos. 5,482,728 or 6,352,734, all of which are incorporated herein by reference.

A food product/precursor in some aspects can be a flour-based or meal-based dough, baked product (bakery product), or extruded product, such as any of those disclosed in WO2021/034561 or U.S. Patent Appl. Publ. Nos. 2017/0218093 or 2022/0322685, which are incorporated herein by reference. Examples of baked products, or a dough (precursor) thereof, include bread (e.g., buns, sourdough, rye, whole wheat, pita, flatbread, tortilla, cornbread, brioche, white, baguette, bagels, banana, ciabatta, brown, challah, focaccia, multigrain, bread sticks, soda bread, pumpernickel, potato bread, biscuits, English muffins, whole grain, matzo, lavash, croutons, pizza crust) (leavened or unleavened), cake (e.g., carrot cake, red velvet, angle food, pound cake, chocolate, white, black forest, tiramisu, coffee cake, cheesecake, devil's food, upside-down cake, Boston cream pie, Swiss roll, lemon cake, short cake, chiffon cake, butter cake, spice cake, rum cake, sponge cake, marble cake, coconut cake, pandan cake), muffins, brownies, scones, cookies, bars, custards, pies, crackers, pretzels, pastries, pudding and tarts. Examples of an extruded product include pasta (e.g., spaghetti, rotini, fusilli, penne, bucatini, macaroni/maccheroni, rigatoni, fettuccine, linguine, vermicelli, ziti, farfalle, gomiti/elbow, rotelle), cereal (e.g., direct expanded cereal, filled cereal, flakes, breakfast cereal), some bread products (e.g., croutons, bread sticks, flat breads), pre-made cookie-dough, dry and semi-moist pet food (e.g., kibbles), and snacks (e.g., cheese curls, filled pillow puffs, chips [e.g., corn chips, pita chips, processed potato chips, tortilla chips], snack sticks [e.g., vegetable sticks], puffed shaped products such as curls [e.g., cheese curls], balls, tubes, bananas, cups, bowls, disks, baby food puffs). Pasta herein can be extruded (e.g., see above) and/or flattened/rolled (e.g., lasagna), fresh or dried, long or short, minute/soup pasta (pastina), filled (e.g., tortellini, ravioli, agnolotti, tortelli), stretched (e.g., cencioni, corzetti, foglie d'ulivo, orecchiette), and/or egg pasta, for example.

A food product/precursor in some aspects can be a syrup or beverage, for example, such as any of those disclosed in U.S. Patent Appl. Publ. Nos. 2010/0040728, 2017/0006902, 2017/0218093, 2013/0216652, 20180146699, 2009/0123603, 2021/0076724, or 2017/0332670, all of which are incorporated herein by reference. A beverage in some aspects can be a juice (e.g., fruit juice such as orange juice, apple juice, mango juice, peach juice, banana juice, date juice, apricot juice, grapefruit juice, papaya juice, pineapple juice, raspberry juice, strawberry juice, pear juice, tangerine juice, or cherry juice; vegetable juice such as carrot juice, tomato juice, or mixed-vegetable juice), sweetened beverage (soda/soft drink, sweetened tea of coffee), ready-to-drink (RTD), or any other beverage having natural and/or added sugar (sucrose).

A food product/precursor in some aspects can be a fermented food product/precursor, for example, such as any of those disclosed in WO2002/034061. In some aspects, a food product/precursor provided in step (a) of a method herein is fermented, while in some aspects a food product/precursor is fermented (or further fermented) during or after performing step (b). Thus, step (a) of a method herein can optionally comprise a step of fermenting a food product/precursor (e.g., before or after adding sucrose, if applicable). Thus, step (b) of a method herein can optionally comprise fermenting the food product/precursor while contacting it with a GTF enzyme. Thus, a method herein can optionally comprise, following step (b), a step of fermenting the food product/precursor. One or more bacterial and/or yeast cultures can be used for fermentation of a food product/precursor herein. Suitable bacteria for food fermentation herein include lactic acid bacteria, for example, such as Lactobacillaceae family species such as those of the Pediococcus genus (e.g., P. acidilactici, P. pentosaceus), Lactobacillus genus (e.g., L. sakei, L. fermentum [formerly L. cellobiosus], L. rhamnosus, L. plantarum, L. brevus, L. kefir, L. casei, L. paracasei, L. acidophilus, L. salivarius, L. buchneri, L. helveticus, L. reuteri, L. johnsonii, L. crispatus, L. gasseri, L. delbruecki such as subsp. L. bulgaricus), Lactococcus genus (e.g., L. lactis such as subsp. L. cremoris), Leuconostoc genus (e.g., L. citreum, L. mesenteroides), and Streptococcus genus (e.g., S. thermophilus). Suitable bacteria for food fermentation in some aspects can be species from the Bifidobacterium genus (e.g., B. bifidum, B. longum, B. animalis, B. breve, B. infantis) or Propionibacterium genus (e.g., P. freudenreichii such as subsp. P. shermanii) (propionic acid bacteria). In some aspects, a bacteria for food fermentation herein can be characterized as Gram-positive, sphere-shaped, rod-shaped, anaerobic, aerobic, acid-tolerant, non-sporulating, GRAS (generally regarded as safe), and/or probiotic. A bacteria for food fermentation (e.g., yogurt or other dairy fermentation) in some aspects can be an acidic culture/strain (or mix) (e.g., (YO-MIX 863, YO-MIX 410, or YO-MIX T42, available from IFF) that produces food with a pH of about, for example, 2.5-4.5, 3.0-4.5, 4.2-4.4, or 4.3, or it can be a mild culture/strain (or mix) (e.g., YO-MIX PRIME 900 or YO-MIX M01, available from IFF) that produces food with a pH of about, for example, 4.6-5.5, 4.6-6.0, 4.5-4.7, or 4.6. A bacteria herein (e.g., mild of acidic) can optionally be mesophilic (temperature for optimal growth typically at 20 to 25° C. [room temperature]). A mix of bacteria in a culture for food fermentation can comprise one, two, three, four, five, six or more different species and/or sub-species of bacteria, for example. Suitable yeast for food fermentation in some aspects include species from the Saccharomyces genus (e.g., S. cerevisiae, S. pastorianus, S. boulardii, S. kluyveri), Pichia genus (e.g., P. kluyveri, P. fermentans), and Candida genus (e.g., C. humilis, C. famata). A yeast in some aspects can be characterized as baker's (baking) yeast, brewing yeast, wine-making yeast, probiotic yeast, budding/fission yeast, or GRAS. A mix of yeast in a culture for food fermentation can comprise one, two, three, four, five, six or more different species and/or sub-species of yeast, for example. A food product/precursor that is fermented or will be fermented can be a dairy product herein (e.g., as above, such as yogurt), beer, beer wort, wine, pomace, cider, miso, kimchi, sauerkraut, pickles/pickle juice, soybean curd, tofu, kombucha, soy sauce, bread, sourdough, or meat, for example.

A food product/precursor in some aspects can be a confectionary, for instance. Examples of confectioneries herein include boiled sugars (hard boiled candies [i.e., hard candy]), dragees, jelly candies, gums, licorice, chews, caramels, toffee, fudge, chewing gums, bubble gums, nougat, chewy pastes, halawa, tablets, lozenges, icing, frosting, pudding, gels (e.g., fruit gels, gelatin dessert), aerated confectioneries, marshmallows, baked confectioneries.

A food product/precursor in some aspects can be a non-dairy food product/precursor. For example, a non-dairy food product/precursor can be a plant-based milk (milk substitute) or comprise a plant-based milk (and lack, or have little of [e.g., <0.5 wt %], any dairy ingredient[s] such as lactose, whey, casein, and/or milk fat). In some aspects, a non-dairy food product/precursor is fermented (e.g., a non-dairy yogurt product/precursor such as a plant-based yogurt product/precursor). Plant-based ingredient(s) forming the basis for a non-dairy food product/precursor herein can be from nuts/seeds (e.g., almonds, cashews, macadamias, hemp seed, quinoa, flax seed), grains/cereal (e.g., oats, rice), fruit (e.g., coconut, banana), or vegetables (e.g., legumes such as beans [e.g., soybeans, mug beans] and peas), for example. In some aspects, a non-dairy food product/precursor is a milk of any of the foregoing nuts/seeds, grains/cereal, fruit, or vegetables; a fermented form of any of these milks can be a yogurt, for example.

A food product/precursor in some aspects can be a cream soup, gravy, sauce (e.g., tomato sauce), salad dressing, mayonnaise, jam, jelly, marmalade, syrup, pie filling, batter for fried foods, batter for pancakes/waffles, cake icing and glazes, whipped topping, pet food, or animal/livestock feed.

A food product/precursor in some aspects can comprise one or more additional ingredients such as a vegetable component (e.g., vegetable oil, vegetable protein, vegetable carbohydrates), enzyme, fat, oil, flavoring agent, microbial culture (e.g., probiotic culture), salt, sweetener, acid, fruit/vegetable (e.g., orange, apple, mango, peach, plum, banana, date, apricot, grapefruit, Papaya, pineapple, raspberry, strawberry, blueberry, blackberry, pear, tangerine, cherry, grape, melon, watermelon, cantaloupe, honeydew melon, kiwi, lemon, lime, carrot, tomato), or fruit/vegetable juice (juice concentrate), purée, or other processed form (e.g., sliced, cubed, chopped pieces) of a fruit/vegetable as disclosed, or any other component suitable for use as an ingredient in a food product/precursor. Such one or more additional ingredients can be as disclosed in U.S. Patent Appl. Publ. Nos. 2016/0122445 or 2017/0218093 (both of which are incorporated herein by reference), for example, and/or can be natural or artificial. Examples of ingredients suitable as sweeteners (or for any other purpose such as flavoring) include acesulfame potassium, advantame, agave syrup, alitame, aspartame, barley malt syrup, birch syrup, brazzein, brown rice syrup, cane juice, caramel, coconut palm sugar, corn syrup, curculin, cyclamate, dextrose, erythritol, fructo-oligosaccharide, fructose (levulose), galactose, glucose (dextrose), glycerol (glycerin), glycyrrhizin, golden syrup, high fructose corn syrup (e.g., HFCS-42, -55, -90), high maltose corn syrup (HMCS), honey, hydrogenated starch hydrolysate (HSH), isomalto-oligosaccharide (IMO), inulin, inverted sugar, isomalt, lactitol, lactose, maltitol, maltodextrin, maltose, mannitol, maple syrup, miraculin, molasses (e.g., blackstrap molasses), monatin, monellin, monk fruit, neohesperidin dihydrochalcone, neotame, palm sugar, pentadin, polydextrose, rapadura, refiners syrup, saccharin, sorbitol (glucitol), Sorghum syrup, Stevia/steviol glycoside (e.g., a rebaudioside such as rebaudioside A, rebaudioside D, or rebaudioside M), sucralose, sugar alcohol, tagatose, thaumatin, trehalose, xylitol, and yacon syrup.

In some aspects, a food product/precursor as produced by a method of the present disclosure can be concentrated, dried (e.g., to a powder), reconstituted (following concentration or drying), or processed (e.g., frozen) in any other manner. Examples of such products include sweetened milk, concentrated milk, condensed milk (e.g., sweetened condensed milk), evaporated milk, dried milk powder, frozen dairy product (e.g., ice cream) concentrated juice, or dried juice powder.

In some aspects, a method herein can further comprise a step of freezing a food product/precursor (e.g., a dairy food product/precursor, or a plant-based food product/precursor) after step (b). This method can produce a frozen dairy product herein such as ice cream or frozen yogurt, or a plant-based ice cream or frozen yogurt, for example. Freezing can be done at about −10, −15, −20, −25, −30, −35, −40, −20 to −40, −25 to −35° C., for example. A frozen product in some aspects—in which method step (b) comprises using at least an alpha-1,3-glucan-producing GTF herein—can have an improved melting profile (e.g., slower melting) as compared to a suitable control (e.g., a frozen product that was not treated with an alpha-1,3-glucan-producing GTF, but otherwise made with the same ingredients and process steps). Slower melting of a frozen product in some aspects can be melting that is reduced by about, or at least about, 10%, 20%, 30%, 40%, 50%, 60%, 70%, 80%, or 90% as compared to the melting of a suitable control. Melting can refer to that melting which occurs within about 45, 60, 75, 90, 100, 120, 150, or 60-120 minutes after placing a frozen product (from freezing conditions) into an ambient temperature (e.g., about 20, 25, 18-25, or 20-25° C.) or an elevated temperature (e.g., about 25-38, 25-35, 25-32, or 25-30° C.), for example. Melting can optionally be as measured according to the below Examples or as disclosed in Granger et al, (2005, Int. Dairy J. 15(3):255-262, incorporated herein by reference), for instance.

A food product/precursor in some aspects after step (b) of a method herein optionally has one or more of the following features as compared to the food product/precursor as it existed before step (b) (i.e. as it existed before being treated with one or more GTF enzymes):
(I) reduced sugar content,
(II) increased texture, such texture optionally comprising increased thickness and/or increased mouthfeel,
(III) improved physical appearance,
(IV) reduced calories,
(V) increased dietary fiber, and/or
(VI) increased stringiness or stretchability.

In some aspects, the sugar content (e.g., wt %) in a food product/precursor after step (b) can be reduced by about, or at least about, 10%, 15%, 20%, 25%, 30%, 35%, 40%, 45%, 50%, 55%, 60%, 65%, 20-65%, 20-60%, 25-65%, or 25-60%, as compared to the sugar content of the food product/precursor as it existed before step (b). In some aspects, this reduction is with respect to all sugars in the food product/precursor, whereas in other aspects this reduction is with respect to a particular sugar such a sucrose. Sugar can be any as presently disclosed, for example. Sugar content herein can be measured by HPLC, for example, such as disclosed in the below Examples.

In some aspects, the texture of a food product/precursor after step (b) can be increased by about, or at least about, 25%, 50%, 75%, 100%, 200%, 300%, 400%, 500%, 750%, 1000%, 1250%, 1500%, 1750%, 2000%, 2250%, 2500%, 3000%, 3500%, 4000%, 4500%, 5000%, 5500%, 6000%, 6500%, or 7000% as compared to the texture of the food product/precursor as it existed before step (b). Texture can be in terms of thickness or mouthfeel, and/or measured in units of Pascal-seconds (Pa.$) or cP, for example, any of which can optionally be measured according to the below Examples. In some aspects, texture (thickness) can be measured by determining food product/precursor viscosity when extracted at a shear rate of about 11 to 12 Hz (e.g., 11.7 Hz). Texture (mouthfeel) can be measured by determining food product/precursor viscosity when extracted at a shear rate of about 248-250 Hz (e.g., 249 Hz), for example.

In some aspects, a food product/precursor after step (b) has an improved physical appearance as compared to the physical appearance of the food product/precursor as it existed before step (b). Improved physical appearance can be increased homogeneity (e.g., visual homogeneity) and/or increased shininess (e.g., visual shininess), for example; such increase(s) can be by about, or at least about, 5%, 10%, 20%, 25%, 30%, 40%, or 50% in some aspects.

In some aspects, the dietary caloric content (calories that can be accessed during digestion) of a food product/precursor after step (b) can be decreased by about, or at least about, 5%, 10%, 20%, 25%, 30%, 40%, 50%, 60%, 70%, or 75% as compared to the dietary caloric content of the food product/precursor as it existed before step (b).

In some aspects, the dietary fiber content (e.g., weight percent) of a food product/precursor after step (b) can be increased by about, or at least about, 5%, 10%, 25%, 50%, 75%, 100%, 200%, 300%, 400%, or 500% as compared to the dietary fiber content of the food product/precursor as it existed before step (b).

In some aspects, such as when contacting a food product/precursor comprising maltose (and/or IMO) (in addition to sucrose) with an alpha-1,3-glucan-producing GTF and/or an alpha-1,6-glucan-producing GTF herein, a high reduction (e.g., at least about 35%, 40%, 45%, 50%, 55%, or 60%) in sugar can be realized without significantly changing the texture (e.g., change less than 10%, 9%, 8%, 7%, 6%, 5%, 4%, 3%, 2%, 1%; e.g., thickness or mouthfeel) of the food product/precursor.

In some aspects, such as when contacting a dairy food product/precursor fermented (or fermenting) with a mild bacterial culture herein with an alpha-1,3-glucan-producing GTF or both an alpha-1,3-glucan-producing GTF and an alpha-1,6-glucan-producing GTF, the sweetness of the dairy food product/precursor is at least 70%, 75%, 80%, 85%, 90%, or 95% that of (i) the sweetness that existed before the contacting with GTF(s), or (ii) the sweetness of a suitable control (e.g., only difference being no GTF treatment), despite complete GTF conversion (e.g., ≥95, 98, or 99 wt %) of the sucrose that was initially present in the dairy food product/precursor. Sweetness can be as measured according to the below Examples, or as disclosed in U.S. Patent Appl. Publ. No. 2009/0053378, for instance, which is incorporated herein by reference.

In some aspects, such as with a frozen dairy product such as ice cream or frozen yogurt, or a plant-based ice cream or frozen yogurt, either optionally lactose-free such as by treatment with a beta-galactosidase, by including (A) both an alpha-1,6-glucan-producing GTF and an alpha-1,3-glucan-producing GTF in step (b), or (B) an alpha-1,3-glucan-producing GTF in step (b) (and the product/precursor is lactose free such as by [co]treating it with a beta-galactosidase), the product hardness can be decreased by about, or at least about, 10%, 15%, 20%, 25%, 30%, 35%, 40%, 45%, 50%, 55%, or 60%, for example, as compared to product made using (i) only an alpha-1,6-glucan-producing GTF in step (b), or (ii) only an alpha-1,3-glucan-producing GTF (under conditions where lactose is present) in step (b). Hardness can be as measured according to the below Examples or as disclosed in WO2009133067 or U.S. Patent Appl. Publ. No. 2016/0366906, for instance, which are incorporated herein by reference.

In some aspects, such as with a dairy food product/precursor (e.g., condensed milk or sweetened condensed milk), contacting a food product/precursor herein with an alpha-1,3-glucan-producing GTF and/or an alpha-1,6-glucan-producing GTF increases the stringiness or stretchability (defined as the vertical stretch a filament of the food product/precursor can sustain before breaking) of the food product/precursor by about, or at least about, 50%, 75%, 100%, 150%, 200%, 250%, 300%, 350%, or 400% as compared to an otherwise same food product/precursor but which was not contacted with either GTF enzyme (all other conditions/parameters being equal). Stringiness/stretchability can be as measured by extensional rheometry in a filament stretching device according to the below Examples, for example. In some aspects, a food product/precursor contacted with an alpha-1,3-glucan-producing GTF and/or an alpha-1,6-glucan-producing GTF can break at a length of about, or at least about, 7, 8, 9, 10, 12.5, 15, 17.5, 20, 22.5, 25, 27.5, 30, 10-30, 10-25, 10-20, 10-15, 12.5-30, 12.5-25, 12.5-20, 12.5-15, 15-30, 15-25, or 15-20 mm (corresponding to the same in seconds where a 1 mm/s is used, such as in the below Examples). Hencky strain versus time (e.g., as measured by extensional rheometry, such as at a rate of ˜1 mm/s) can be measured, for example, according to the below Examples or as disclosed in U.S. Patent Appl. Publ. No. 2009/0246433, or U.S. Pat. Nos. 6,578,413 or 6,711,941, which are incorporated herein by reference.

Some aspects of the present disclosure concern a food product/precursor as produced by a GTF treatment method herein. Examples of such products/precursors are any food product/precursor as disclosed herein. Typically, such a food product/precursor can have any feature as disclosed herein (e.g., reduced sugar content, increased texture, increased stringiness (stretchability), improved physical appearance, reduced caloric content, increased dietary fiber, pH, temperature, age, hardness, reduced melt rate), as appropriate/applicable. Typically, such a food product/precursor comprises at least one GTF enzyme as presently disclosed, and/or an alpha-glucan as presently disclosed.

In some aspects, a disaccharide and/or oligosaccharide (and/or a monosaccharide) herein serves as an acceptor/primer for in situ alpha-glucan synthesis by a GTF enzyme in the food/precursor. Thus, some aspects of the present disclosure concern a polysaccharide or alpha-glucan molecule comprising at least:
(i) alpha-1,6-glucan or alpha-1,3-glucan as disclosed herein, and
(ii) a disaccharide or oligosaccharide as disclosed herein (e.g., maltose, isomaltose, lactose, lactosucrose, MOS, IMO, or GOS),
wherein portion (i) is in glycosidic linkage with portion (ii), and portion (ii) is at the reducing end of the alpha-glucan molecule (e.g., by virtue of having used any of the disaccharides or oligosaccharides of (ii) as an acceptor for priming synthesis of the alpha-1,6-glucan or alpha-1,3-glucan), optionally wherein the alpha-glucan in comprised in any food product/precursor as disclosed herein. The DP or DPw of the alpha-glucan can be any DP or DPw value disclosed herein, for example. In some aspects, such an alpha-glucan can be in the form of and/or comprised in a household care product, personal care product, industrial product, ingestible product (e.g., food product/precursor such as any disclosed herein), or pharmaceutical product, for example, such as described in any of U.S. Patent Appl. Publ. Nos. 2018/0022834, 2018/0237816, 2018/0230241, 20180079832, 2016/0311935, 2016/0304629, 2015/0232785, 2015/0368594, 2015/0368595, 2016/0122445, 2019/0202942, or 2019/0309096, or International Patent Appl. Publ. No. WO2016/133734, which are all incorporated herein by reference. In some aspects, a composition can comprise at least one component/ingredient of a household care product, personal care product, industrial product, pharmaceutical product, or ingestible product (e.g., food product) as disclosed in any of the foregoing publications and/or as presently disclosed.

Some aspects of the present disclosure concern a method of glucosylating a steviol glycoside, which method can optionally be characterized as a steviol glycoside glucosylation method. Such a method can comprise a step of providing a composition that comprises at least water, sucrose, a steviol glycoside, and a glucosyltransferase enzyme, wherein the steviol glycoside comprises, or consists of, stevioside or rebaudioside A, and wherein the glucosyltransferase enzyme is selected from:
(i) a glucosyltransferase enzyme that synthesizes alpha-1,6-glucan, wherein at least about 50% of the glycosidic linkages of the alpha-1,6-glucan are alpha-1,6 linkages, and/or
(ii) a glucosyltransferase enzyme that synthesizes alpha-1,3-glucan, wherein at least about 50% of the glycosidic linkages of the alpha-1,3-glucan are alpha-1,3 linkages,
wherein at least one glucosylated form of the steviol glycoside (glucosylated steviol glycoside) is produced in the composition. The providing step of such a method can optionally be characterized as a step of contacting a steviol glycoside with a glucosyltransferase enzyme in the presence of at least water and sucrose. Typically, a steviol glycoside glucosylation method also produces at least one alpha-glucan as presently disclosed, such as an alpha-1,6-glucan, an alpha-1,3-glucan, and/or a graft copolymer herein. Stevioside herein has the structure of CAS (Chemical Abstracts Service) Registry no. 57817-89-7 and CHEBI (Chemical Entities of Biological Interest) no. 9271, and rebaudioside A herein has the structure of CAS Registry no. 58543-16-1 and CHEBI no. 145012; these database reference numbers and their structures are incorporated herein by reference.

An alpha-1,6-glucan-synthesizing glucosyltransferase enzyme in a steviol glycoside glucosylation method can be any as presently disclosed herein. An alpha-1,3-glucan-synthesizing glucosyltransferase enzyme in a steviol glycoside glucosylation method can be any as presently disclosed herein. Any condition and/or parameter for using any of these enzymes in this method can be as presently disclosed herein (e.g., temperature, time, water content, sucrose content, GTF enzyme content).

The content of one or both of the steviol glycosides stevioside and rebaudioside A in a composition for glucosylation herein can be about, or at least about, 0.001, 0.002, 0.003, 0.004, 0.005, 0.006, 0.007, 0.008, 0.009. 0.01, 0.025, 0.05, 0.075, 0.1, 0.001-0.1, 0.001-0.05, 0.001-0.01, 0.002-0.1, 0.002-0.05, 0.002-0.01, 0.003-0.1, 0.003-0.05, 0.003-0.01, 0.004-0.1, 0.004-0.05, 0.004-0.01, 0.005-0.1, 0.005-0.05, or 0.005-0.01 wt %, for example. The concentration of one or both of the steviol glycosides stevioside and rebaudioside A in a composition for glucosylation herein can be about, or at least about, 0.1, 0.5, 1.0, 1.5, 2.0, 2.5, 3.0, 4.0, 5.0, 0.1-5.0, 0.1-2.5, 0.1-2.0, 0.1-1.5, 0.1-1.0, 0.5-5.0, 0.5-2.5, 0.5-2.0, 0.5-1.5, or 0.5-1.0 mM, for example. Any of the foregoing content/concentration values/ranges can optionally also characterize the content/concentration of a glucosylated steviol glycoside in a composition herein. Stevioside and/or rebaudioside A can be present in a composition for glucosylation herein, for example, by virtue of the composition comprising a Stevia sweetener, Stevia extract, and/or other preparation from (or derivable from) a Stevia rebaudiana plant (particularly, from leaves thereof).

In some additional or alternative aspects, it is believed that one or more of the steviol glycosides dulcoside A, rebaudioside B, rebaudioside C, rebaudioside E, rebaudioside F, rubusoside, and steviolbioside, which could be present in a composition as presently disclosed (e.g., as components of a Stevia extract included in a composition), can be targeted for glucosylation by a glucosyltransferase herein. Thus, for example, insofar as would be considered suitable by a skilled artisan, the term(s) “stevioside” and/or “rebaudioside A” as used in the present disclosure can optionally be replaced with one or more of the terms “dulcoside A”, “rebaudioside B”, “rebaudioside C”, “rebaudioside E”, “rebaudioside F”, “rubusoside”, or “steviolbioside”.

In some aspects, a composition in which a glucosylated steviol glycoside can be produced in a steviol glycoside glucosylation method can be any as presently disclosed herein, such as a food precursor/product. Yet, in some aspects, such a composition can be in the form of, and/or comprised in, a household care product, personal care product, industrial product, ingestible product (e.g., food product/precursor such as any disclosed herein), or pharmaceutical product, for example, such as described in any of U.S. Patent Appl. Publ. Nos. 2018/0022834, 2018/0237816, 2018/0230241, 20180079832, 2016/0311935, 2016/0304629, 2015/0232785, 2015/0368594, 2015/0368595, 2016/0122445, 2019/0202942, or 2019/0309096, or International Patent Appl. Publ. No. WO2016/133734, which are all incorporated herein by reference. In some aspects, a composition can comprise at least one component/ingredient of a household care product, personal care product, industrial product, pharmaceutical product, or ingestible product (e.g., food product) as disclosed in any of the foregoing publications and/or as presently disclosed. A composition of the present disclosure that comprises a glucosylated steviol glycoside (e.g., one comprising a glucosylated steviol glycoside produced by a steviol glycoside glucosylation method herein) can be any of the foregoing compositions/products, for example.

Some aspects of a steviol glycoside glucosylation method comprise fermenting a food product/precursor after the step of providing a food product/precursor that comprises at least water, sucrose, a steviol glycoside, and a glucosyltransferase enzyme. Thus, in such a method, a process for fermenting the food product/precursor is only commenced after some of (e.g., about, or at least about, 30, 40, 50, 60, 70, 80, 90, 95, 98, 99, or 99.5 percent by weight of), or all of, the stevioside and/or rebaudioside A that was initially present in the food product/precursor has been glucosylated by the one or more glucosyltransferase enzymes.

Glucosylated stevioside and/or glucosylated rebaudioside A herein (e.g., product[s] of a steviol glycoside glucosylation method) are believed to comprise 1, 2, 3, 4, 5, 6, 7, 8, 9, 10, or more glucose monomeric units (typically in a chain) added to the original stevioside and/or rebaudioside A molecule, for example. Stevioside and/or rebaudioside A can optionally be characterized herein to serve as an acceptor/primer for alpha-glucan synthesis by a GTF enzyme in a composition of the disclosure. Thus, some aspects of the present disclosure concern a polysaccharide or alpha-glucan molecule comprising at least:
(i) alpha-1,6-glucan or alpha-1,3-glucan as disclosed herein, and
(ii) stevioside or rebaudioside A;
wherein portion (i) is in glycosidic linkage with portion (ii), and portion (ii) is at the reducing end of the alpha-glucan molecule (e.g., by virtue of having used the stevioside or rebaudioside A of (ii) as an acceptor for priming synthesis of the alpha-1,6-glucan or alpha-1,3-glucan). The DP or DPw of the alpha-glucan can be any DP or DPw value disclosed herein, for example.

Some embodiments disclosed herein concern a polynucleotide comprising a nucleotide sequence that encodes a modified (non-native) GTF enzyme as presently disclosed, such as one that has enhanced performance and/or stability benefit(s). Optionally, one or more regulatory sequences are operably linked to the nucleotide sequence, and preferably a promoter sequence is included as a regulatory sequence.

A polynucleotide comprising a nucleotide sequence encoding a modified GTF enzyme herein can be a vector or construct useful for transferring a nucleotide sequence into a cell, for example. Examples of a suitable vector/construct can be selected from a plasmid, yeast artificial chromosome (YAC), cosmid, phagemid, bacterial artificial chromosome (BAC), virus, or linear DNA (e.g., linear PCR product). A polynucleotide sequence in some aspects can be capable of existing transiently (i.e., not integrated into the genome) or stably (i.e., integrated into the genome) in a cell. A polynucleotide sequence in some aspects can comprise, or lack, one or more suitable marker sequences (e.g., selection or phenotype marker).

A polynucleotide sequence in some aspects can comprise one or more regulatory sequences operably linked to the nucleotide sequence encoding a modified GTF enzyme. For example, a nucleotide sequence encoding a modified GTF enzyme can be in operable linkage with a promoter sequence (e.g., a heterologous promoter). A promoter sequence can be suitable for expression in a cell (e.g., bacterial cell such as E. coli or Bacillus; eukaryotic cell such as a fungus, yeast, insect, or mammalian cell) or in an in vitro protein expression system, for example. Examples of other suitable regulatory sequences include transcription terminator sequences.

Some aspects herein are drawn to a cell comprising a polynucleotide sequence as presently disclosed; such a cell can be any type disclosed herein (e.g., bacterial cell such as E. coli or Bacillus; eukaryotic cell such as a fungus, yeast, insect, or mammalian cell). A cell can optionally express a modified GTF enzyme encoded by the polynucleotide sequence. In some aspects, the polynucleotide sequence exists transiently (i.e., not integrated into the genome) or stably (i.e., integrated into the genome) in the cell.

Non-limiting examples of compositions and methods disclosed herein include:
1. A method of producing a food product/precursor, the method comprising: (a) providing a food product/precursor that comprises at least water and sucrose, and (b) contacting the food product/precursor with at least: (i) a glucosyltransferase enzyme that synthesizes alpha-1,6-glucan, wherein at least about 50% of the glycosidic linkages of the alpha-1,6-glucan are alpha-1,6 linkages, and (ii) a glucosyltransferase enzyme that synthesizes alpha-1,3-glucan, wherein at least about 50% of the glycosidic linkages of the alpha-1,3-glucan are alpha-1,3 linkages, wherein at least one alpha-glucan is produced in the food product/precursor, whereby the food product/food precursor, after step (b), optionally has one or more of the following features as compared to the food product/precursor before step (b) (or a suitable control, such as a product/precursor that only differs by not being contacted with the GTF enzymes): (I) reduced sugar content, (II) increased texture, the texture optionally comprising increased thickness and/or increased mouthfeel, (Ill) improved physical appearance (e.g., increased homogeneity and/or shininess), (IV) reduced calories, (V) increased dietary fiber, and/or (VI) increased stringiness or stretchability.
2. The method of embodiment 1, wherein: the glucosyltransferase enzyme that synthesizes alpha-1,6-glucan comprises an amino acid sequence that is at least 90% identical to SEQ ID NO:1, 2, 11, or 12, and/or the glucosyltransferase enzyme that synthesizes alpha-1,3-glucan comprises an amino acid sequence that is at least 90% identical to residues 55-960 of SEQ ID NO:5, residues 54-957 of SEQ ID NO:6, residues 55-960 of SEQ ID NO:7, residues 55-960 of SEQ ID NO:8, residues 55-960 of SEQ ID NO:9, or SEQ ID NO:13.
3. The method of embodiment 1 or 2, wherein the alpha-glucan produced in step (b) comprises a graft copolymer comprising: (i) an alpha-1,6-glucan backbone, wherein at least about 50% of the glycosidic linkages of the alpha-1,6-glucan backbone are alpha-1,6 linkages, and (ii) at least one alpha-1,3-glucan side chain, wherein at least about 50% of the glycosidic linkages of the alpha-1,3-glucan chain are alpha-1,3 linkages, wherein the alpha-glucan is aqueous-soluble or aqueous-insoluble.
4. The method of embodiment 1, 2, or 3, wherein the ratio of the glucosyltransferase enzyme that synthesizes alpha-1,6-glucan to the glucosyltransferase enzyme that synthesizes alpha-1,3-glucan in step (b) is about 85:15 to about 95:5.
5. The method of embodiment 1, 2, 3, or 4, wherein step (a) comprises adding sucrose to the food product/precursor.
6. The method of embodiment 1, 2, 3, 4, or 5, wherein the food product/precursor provided in step (a) further comprises at least one disaccharide in addition to the sucrose, and/or at least one oligosaccharide.
7. The method of embodiment 6, wherein the disaccharide is lactose or maltose, and the oligosaccharide is galacto-oligosaccharide, isomalto-oligosaccharide, or malto-oligosaccharide.
8. The method of embodiment 6 or 7, wherein the oligosaccharide is provided in the food product/precursor provided in step (a) by contacting the food product/precursor with a transglucosidase or a transgalactosylating beta-galactosidase.
9. The method of embodiment 1, 2, 3, 4, or 5, wherein the food product/precursor provided in step (a) has few or no disaccharides and/or oligosaccharides, aside from the sucrose.
10. The method of embodiment 1, 2, 3, 4, 5, 6, 7, 8, or 9, wherein step (a) comprises contacting the food product/precursor with a glycosidase, optionally wherein the glycosidase is a beta-galactosidase.
11. The method of embodiment 1, 2, 3, 4, 5, 6, 7, 8, 9, or 10, wherein the food product/precursor is a dairy food product/precursor.
12. The method of embodiment 1, 2, 3, 4, 5, 6, 7, 8, 9, 10, or 11, wherein the food product/precursor of step (a) is fermented, or the method further comprises, during or after step (b), fermenting the food product/precursor.
13. The method of embodiment 1, 2, 3, 4, 5, 6, 7, 8, 9, 10, 11, or 12, wherein the food product/precursor is: (i) a dairy food product/precursor, or (ii) a non-dairy food product/precursor, optionally wherein the non-dairy food product/precursor is plant-based, optionally wherein the food product/precursor is a yogurt product/precursor (e.g., a non-dairy yogurt product/precursor, or a plant-based yogurt product/precursor).
14. The method of embodiment 12 or 13, wherein a mild culture strain is used to ferment the food product/precursor.
15. The method of embodiment 11, 12, 13, or 14, further comprising freezing the dairy food product/precursor after step (b), optionally wherein the method produces ice cream.
16. The method of embodiment 1, 2, 3, 4, 5, 6, 7, 8, 9, 10, 11, 12, 13, 14, or 15, wherein step (b) comprises contacting the food product/precursor with an aqueous composition comprising at least sucrose, the glucosyltransferase enzyme that synthesizes alpha-1,6-glucan, and the glucosyltransferase enzyme that synthesizes alpha-1,3-glucan, wherein said aqueous composition is incubated for at least about 10 minutes before the contacting (optionally, in some other aspects, the food product/precursor instead lacks sucrose and/or water).
17. The method of embodiment 16, wherein the initial sucrose concentration of the aqueous composition is about 5 to 60 wt %.
18. A food product/precursor produced by the method of embodiment 1, 2, 3, 4, 5, 6, 7, 8, 9, 10, 11, 12, 13, 14, 15, 17, 19, 20, or 21 and/or that comprises an alpha-glucan molecule according to embodiment 22, and/or that comprises a modified glucosyltransferase according to embodiment 23.
19. A method according to embodiment 13, 14, or 15, wherein the food product/precursor is the non-dairy food product/precursor, optionally wherein the non-dairy food product/precursor is plant-based, optionally wherein the food product/precursor is a yogurt product/precursor (e.g., a non-dairy yogurt product/precursor, or a plant-based yogurt product/precursor), but wherein step (b) instead comprises contacting the food product/precursor with at least: (i) a glucosyltransferase enzyme that synthesizes alpha-1,6-glucan, wherein at least about 50% of the glycosidic linkages of the alpha-1,6-glucan are alpha-1,6 linkages, or (ii) a glucosyltransferase enzyme that synthesizes alpha-1,3-glucan, wherein at least about 50% of the glycosidic linkages of the alpha-1,3-glucan are alpha-1,3 linkages (instead of having to use both [i] and [ii]).
20. A method according to embodiment 15, but wherein step (b) instead comprises contacting the food product/precursor with at least: (i) a glucosyltransferase enzyme that synthesizes alpha-1,6-glucan, wherein at least about 50% of the glycosidic linkages of the alpha-1,6-glucan are alpha-1,6 linkages, or (ii) a glucosyltransferase enzyme that synthesizes alpha-1,3-glucan, wherein at least about 50% of the glycosidic linkages of the alpha-1,3-glucan are alpha-1,3 linkages (instead of having both [i] and [ii]).
21. A method according to embodiment 1, 2, 3, 4, 5, 6, 7, 8, 9, 10, 11, 12, 13, 14, 15, 16, 17, 19, or 20, but wherein step (b) instead comprises contacting the food product/precursor with at least: (i) a modified (non-native) glucosyltransferase enzyme that synthesizes alpha-1,3-glucan, wherein at least about 50% of the glycosidic linkages of the alpha-1,3-glucan are alpha-1,3 linkages, wherein the modified glucosyltransferase enzyme comprises one or more amino acid substitution(s) at a position(s) corresponding with amino acid residue(s) Tyr-185, Val-186, Leu-513, Gln-588, Phe-607, Ile-608, Lys-625, Arg-741, Val-1188, Lys-1327, Glu-1332, Asp-1418, Ala-1419, Ser-1420, Thr-1421, Arg-1424, Leu-1425, Thr-1431, and/or Glu-1450 of SEQ ID NO:10, and optionally wherein the modified glucosyltransferase comprises an amino acid sequence that is at least about 90% identical to SEQ ID NO:5, 6, 7, 8, or 9, or amino acid residues 55-960 of SEQ ID NO:5, amino acid residues 54-957 of SEQ ID NO:6, amino acid residues 55-960 of SEQ ID NO:7, amino acid residues 55-960 of SEQ ID NO:8, or amino acid residues 55-960 of SEQ ID NO:9, optionally wherein the modified glucosyltransferase has enhanced performance and/or stability benefit(s), and optionally (ii) a glucosyltransferase enzyme that synthesizes alpha-1,6-glucan, wherein at least about 50% of the glycosidic linkages of the alpha-1,6-glucan are alpha-1,6 linkages.
22. An alpha-glucan molecule comprising at least: (i) alpha-1,6-glucan or alpha-1,3-glucan as disclosed herein, and (ii) maltose, isomaltose, malto-oligosaccharide (MOS), isomalto-oligosaccharide (IMO), or galacto-oligosaccharide (GOS); wherein portion (i) is in glycosidic linkage with portion (ii), and portion (ii) is at the reducing end of the alpha-glucan molecule (e.g., such alpha-glucan molecule can be produced by using any of the disaccharides or oligosaccharides of (i) as an acceptor for priming synthesis of the alpha-1,6-glucan or alpha-1,3-glucan), optionally wherein the alpha-glucan in comprised in any food product/precursor as disclosed herein.
23. A modified (non-native) glucosyltransferase enzyme that synthesizes alpha-1,3-glucan, wherein at least about 50% of the glycosidic linkages of the alpha-1,3-glucan are alpha-1,3 linkages, wherein the modified glucosyltransferase enzyme comprises one or more amino acid substitution(s) at a position(s) corresponding with amino acid residue(s) Tyr-185, Val-186, Leu-513, Gln-588, Phe-607, Ile-608, Lys-625, Arg-741, Val-1188, Lys-1327, Glu-1332, Asp-1418, Ala-1419, Ser-1420, Thr-1421, Arg-1424, Leu-1425, Thr-1431, and/or Glu-1450 of SEQ ID NO:10, and optionally wherein the modified glucosyltransferase comprises an amino acid sequence that is at least about 90% identical to SEQ ID NO:5, 6, 7, 8, or 9, or amino acid residues 55-960 of SEQ ID NO:5, amino acid residues 54-957 of SEQ ID NO:6, amino acid residues 55-960 of SEQ ID NO:7, amino acid residues 55-960 of SEQ ID NO:8, or amino acid residues 55-960 of SEQ ID NO:9, optionally wherein the modified glucosyltransferase has enhanced performance and/or stability benefit(s).

Non-limiting examples of compositions and methods disclosed herein include:
1a. A method of glucosylating a steviol glycoside, the method comprising: providing a composition that comprises at least water, sucrose, a steviol glycoside, and a glucosyltransferase enzyme (or contacting a steviol glycoside with a glucosyltransferase enzyme in the presence of at least water and sucrose), wherein the steviol glycoside comprises stevioside or rebaudioside A, and wherein the glucosyltransferase enzyme is selected from: (i) a glucosyltransferase enzyme that synthesizes alpha-1,6-glucan, wherein at least about 50% of the glycosidic linkages of the alpha-1,6-glucan are alpha-1,6 linkages, and/or (ii) a glucosyltransferase enzyme that synthesizes alpha-1,3-glucan, wherein at least about 50% of the glycosidic linkages of the alpha-1,3-glucan are alpha-1,3 linkages,
wherein at least one glucosylated form of the steviol glycoside (glucosylated steviol glycoside) is produced in the composition, and typically wherein at least one alpha-glucan (e.g., the alpha-1,6-glucan, the alpha-1,3-glucan, and/or a graft copolymer herein) is produced in the composition.
2a. The method of embodiment 1a, wherein: the glucosyltransferase enzyme that synthesizes alpha-1,6-glucan comprises an amino acid sequence that is at least 90% identical to SEQ ID NO:1, 2, 11 or 12, and/or the glucosyltransferase enzyme that synthesizes alpha-1,3-glucan comprises an amino acid sequence that is at least 90% identical to residues 55-960 of SEQ ID NO:5, residues 54-957 of SEQ ID NO:6, residues 55-960 of SEQ ID NO:7, residues 55-960 of SEQ ID NO:8, residues 55-960 of SEQ ID NO:9, or SEQ ID NO:13.
3a. The method of embodiment 1a or 2a, wherein the alpha-glucan is produced in the composition.
4a. The method of embodiment 1a, 2a, or 3a, wherein the glucosyltransferase enzymes of (i) and (ii) are included in the composition, optionally wherein: (A) the ratio of the glucosyltransferase enzyme of (i) to the glucosyltransferase enzyme of (ii) in the composition is about 85:15 to about 95:5, and/or (B) the graft copolymer is produced in the composition.
5a. The method of embodiment 1a, 2a, 3a, or 4a, wherein the composition is a food product/precursor.
6a. The method of embodiment 5a, wherein the food product/precursor is a dairy food product/precursor, optionally wherein the food product/precursor is a yogurt product/precursor.
7a. The method of embodiment 5a or 6a, further comprising: fermenting the food product/precursor after the providing step (or fermenting the food product/precursor after contacting the steviol glycoside with the glucosyltransferase enzyme in the presence of at least water and sucrose).
8a. The method of embodiment 1a, 2a, 3a, 4a, 5a, 6a, or 7a, wherein the composition comprises the steviol glycoside by virtue of comprising a Stevia sweetener, Stevia extract, and/or other preparation from a Stevia rebaudiana plant (particularly, from leaves thereof), typically whereby the composition further comprises one or more steviol glycosides selected from dulcoside A, rebaudioside B, rebaudioside C, rebaudioside D, rebaudioside E, rebaudioside F, rebaudioside M, rubusoside, and/or steviolbioside.
9a. The method of embodiment 1a, 2a, 3a, 4a, 5a, 6a, 7a, or 8a, whereby the composition, after the providing step (or the contacting step), has one or more of the following features as compared to the composition before the providing step (or the contacting step): (I) reduced sugar content, (II) increased texture, said texture optionally comprising increased thickness and/or increased mouthfeel, (Ill) improved physical appearance, said appearance optionally being increased homogeneity and/or shininess, and/or (IV) increased stringiness or stretchability.
10a. A composition, or a glucosylated steviol glycoside, produced by the method of embodiment 1a, 2a, 3a, 4a, 5a, 6a, 7a, 8a, or 9a.
11a. A composition comprising a glucosylated steviol glycoside, wherein the glucosylated steviol glycoside is produced by contacting a steviol glycoside with a glucosyltransferase enzyme in the presence of at least water and sucrose, wherein the steviol glycoside comprises stevioside or rebaudioside A, and wherein the glucosyltransferase enzyme is selected from: (i) a glucosyltransferase enzyme that synthesizes alpha-1,6-glucan, wherein at least about 50% of the glycosidic linkages of the alpha-1,6-glucan are alpha-1,6 linkages, and/or (ii) a glucosyltransferase enzyme that synthesizes alpha-1,3-glucan, wherein at least about 50% of the glycosidic linkages of the alpha-1,3-glucan are alpha-1,3 linkages, optionally wherein the composition is a food product/precursor.
12a. A method of embodiment 1a, 2a, 3a, 4a, 5a, 6a, 7a, 8a, or 9a, or a composition of embodiment 10a or 11a, but wherein stevioside and/or rebaudioside A is/are replaced with, or supplemented with, one or more of the steviol glycosides of dulcoside A, rebaudioside B, rebaudioside C, rebaudioside E, rebaudioside F, rubusoside, or steviolbioside (typically, one or more of these can be glucosylated by the glucosyltransferase enzyme).

### EXAMPLES

The present disclosure is further exemplified in the following Examples. It should be understood that these Examples, while indicating certain aspects herein, are given by way of illustration only. From the above discussion and these Examples, one skilled in the art can ascertain the essential characteristics of the disclosed embodiments, and without departing from the spirit and scope thereof, can make various changes and modifications to adapt the disclosed embodiments to various uses and conditions.

### Materials/Methods

### HPLC Sugar Content Analysis

Sugar composition was measured by high performance liquid chromatography (HPLC) with a Waters® 2695 Serrations module or a ThermoScientific Dionex™ UltiMate 3000 HPLC, equipped with a Phenomonex Rezex™ RPM-Monosaccharide Pb²⁺ column (300 mm×7.8 mm), and an RI-detector. Water was used as the mobile phase at a flow rate of 0.400 mL/min. The column temperature was 70° C. Samples were prepared for HPLC injection by an appropriate dilution in water, optionally centrifugation (10 min at 15,000 rpm), and a sterile filtration. Signals from the HPLC were quantified against calibration standards of sugars eluting at the same time. Sugar reduction was calculated by subtracting the total sum of mono- and di-saccharides in the test sample from the total sum of mono- and di-saccharides in the reference sample without enzymes added.

### Yogurt Preparation

Pre-pasteurized (72° C. for 15 s) bulk-blended skimmed milk (0.1% fat) (Arta Foods, Denmark) stored at 4-6° C. was standardized to a desired protein (% w/w), fat (% w/w) and sucrose (% w/w) content by addition of skimmed milk powder (33% protein, 1.2% fat, 54% carbohydrate) from BBA Lactalis (Laval, Mayenne, France), cream (38% fat) from Arla Foods, and sucrose (Granulated Sugar 500, Nordic Sugar A/S, Denmark). The thus prepared standardized milk was then pasteurized and homogenized in a plate heat exchange pasteurizer. Homogenization was performed at 65° C. at 200 bar and pasteurization at 95° C. for 6 minutes, and then the milk was cooled to 43° C. The milk was inoculated with a thermophilic starter culture at an inoculation rate of 20 DCU/1001_, all cultures were from IFF. Fermentation was conducted until pH 4.60 after which the product was cooled to 24° C. The resulting yogurts were stored at 4-6° C. for viscosity measurements.

### Method for Measuring Apparent Viscosity

A rotational rheological test was employed to evaluate the viscosity of the produced samples. Flow curves were obtained with an Anton Paar MCR302 rheometer (Anton Paar GmbH, Ostfildern, Germany) using an ST22-4V-40 vane geometry for alu cups. Samples were filled into C-CC27 alu cups and stored at 5° C. for at least 5 hours before analysis. The shear rate intervals applied to the samples were 0.1-350 s⁻¹, which defines the up-curve, and the reverse operation explains the down-curve (350-0.1 s⁻¹). The value of the measuring point duration was selected to be at least as long as the value of the reciprocal shear rate, which is valid for the up-curve. The tests were performed under a constant temperature of 10° C., and each sample was analyzed in duplicate. A water bath was connected to the rheometer to ensure isothermal conditions.

The apparent viscosity was assessed from the flow curves, which is appropriate for fluids where the ratio of shear stress to shear rate varies with the shear rate. The apparent viscosity was extracted at either shear rate 11.7 Hz or 249 Hz. The apparent viscosity extracted at shear rate 11.7 Hz indicated the “thickness” of the sample. The apparent viscosity extracted at shear rate 249 s⁻¹ (249 Hz) was correlated to the sensory perception of “mouthfeel”.

### Measuring Physical Features of Ice Cream

Ice cream melting stability was analyzed according to Granger et al. (2005, Int. Dairy J. 15(3):255-262, incorporated herein by reference), with slight modifications. A block of pre-weight ice cream (60-70 g) was put on a net (mash size of 0.5 cm×0.5 cm) at 20° C. The net with the ice cream was placed above a scale with a beaker. During melting of the ice cream, liquid dripped though the net into the beaker placed on a scale. The liquid in the beaker was weighed every 10 seconds. Melt-down was defined as the percentile of the liquid in the beaker at a given timepoint, divided by the initial weight of the ice cream on the net at the start of the measurement. Also, the time between placing the ice cream sample in the net to the time the first drop of melted ice cream hit the beaker was recorded (first drop).

Ice cream texture was analyzed according to Parvar et al. (2013, Food Biosci. 3:10-18, incorporated herein by reference), with minor modification. Analysis was performed using a TA.XTPLUS Texture Analyzer equipped with a P110 cylindric measurement probe (Stable Micro System, Surrey, United Kingdom). An ice cream block (0.24 L; 10.4 cm×5.4 cm×4.4 cm) was penetrated with the texture analyzer at a speed of 2 mm s⁻¹ to a depth of 15 mm. The probe was retracted from the ice cream at a speed of 2 mm s⁻¹, The block was measured at three spots (center, 1.5 cm left of center, 1.5 cm right of center). The hardness and cohesiveness of ice cream were defined as, respectively, the maximal force needed to penetrate (maximal positive force) or extract (maximal negative force) the probe from the ice cream sample. The adhesiveness of ice cream was defined as the negative area when plotting the force over the analysis time. All analyses were performed at a constant ice cream temperature (˜15 C±2° C.). Since each analysis took less than a minute, no external sample cooling was needed.

### Ultra-High Performance Liquid Chromatography (UHPLC)

UHPLC was performed following methodology disclosed in Gardana et al, (2018, Journal of Chromatography A 1578:8-14), which is incorporated herein by reference. The analysis of different steviol glycosides was carried out on an Agilent 1290 UHPLC system (containing a 1290Bio multisampler, 1290 binary pump, 1290 thermostated column compartment and a 1260 diode array detector). A Waters Acquity UPLC BEH amide column (150×3.0 mm id, 1.7 μm) was used for the separation. The column was maintained at 35° C. and the samples at 15° C. The mobile phases were (A) 0.05% formic acid in water and (B) 0.05% formic acid in acetonitrile. The flow rate was 0.3 mL/min and the gradient was (% A/min): 10/0, 35/20, 50/20.5, 21/50, 10/21.5, or 10/25. Steviol glycosides were detected using a diode array detector at 200 nm.

Standards of stevioside, rebaudioside A, rebaudioside B, rebaudioside C, rebaudioside D, rebaudioside E, rebaudioside F, rebaudioside M, rebaudioside N, rubusoside and dulcoside A were dissolved in methanol (approx. 1 mg/mL) and diluted with water to be used as retention time standards.

Samples were centrifuged and the upper phase was transferred to an HPLC vial and analysed directly by the UHPLC method.

### Example 1

### In Situ-Produced Dextran-Alpha-1,3-Glucan Graft Copolymer in Yogurt

In this Example, a graft copolymer having a dextran backbone with alpha-1,3-glucan side chains was produced in yogurt in situ using a combination of a dextransucrase and an alpha-1,3-glucan sucrase. This glucan production coincided with sugar reduction and texturizing effects in the yogurt.

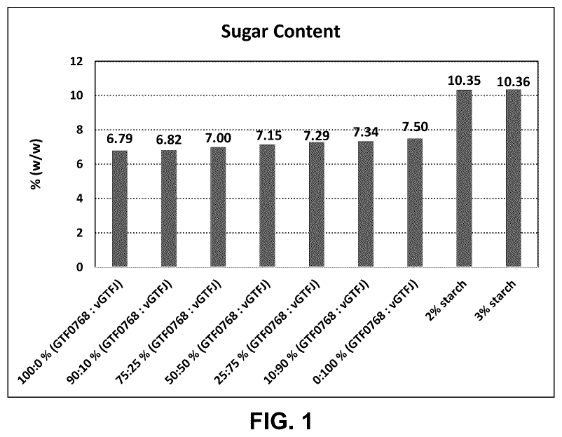

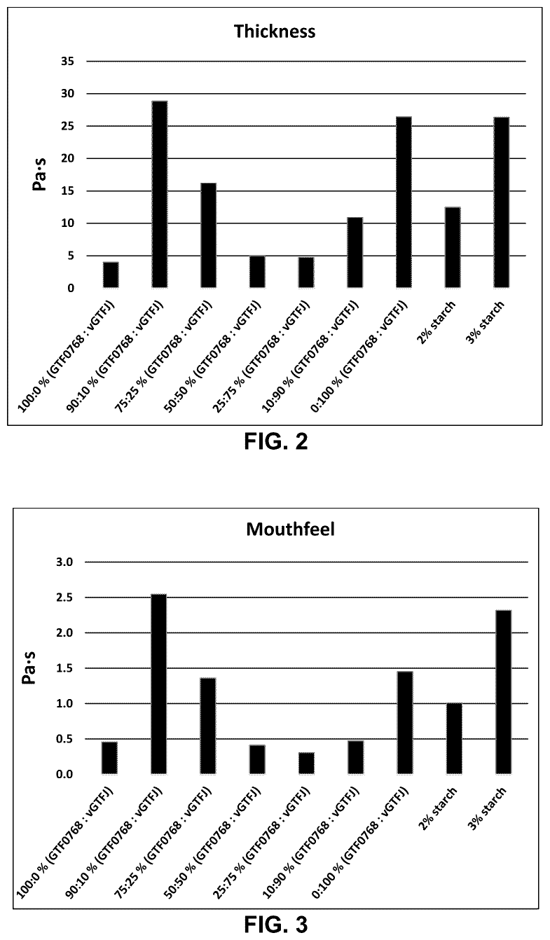

The sugar reduction and texturing effects of using glucosyltransferase (GTF) 0768 (SEQ ID NO:1, also represented by SEQ ID NOs:2, 11 and 12) and an amino acid-substituted GTF 6855 variant (SEQ ID NO:3, “vGTFJ” herein) alone, or in combination, was investigated in a yogurt production set-up (scale: 60 g). Both these GTF enzymes use sucrose as a substrate to produce fructose and glucan (i.e., they are glucansucrases). GTF 0768 (a dextransucrase) produces a soluble alpha-glucan with a high alpha-1,6 linkage content (i.e., a type of dextran; refer to U.S. Patent Appl. Publ. No. 2016/0122445, which is incorporated herein by reference), whereas vGTFJ stably produces at high yield an insoluble alpha-glucan having about 100% alpha-1,3 linkages (the variant GTF of SEQ ID NO:4 similarly can be used herein to stably produce at high yield insoluble alpha-glucan having about 100% alpha-1,3 linkages). Fresh milk standardized to 4.0% (w/w) protein, 1.0% (w/w) fat and 6.0% (w/w) sucrose was homogenized and pasteurized as described in the Materials/Methods. GTF enzymes were then added at the inoculation step as presented in Table 1. 100% GTF 0768 or 100% vGTFJ is the amount of each respective enzyme sample needed to fully convert the sucrose within the time of the fermentation. YO-MIX 410 was employed as starter culture (available from IFF). After 3 days of storage at 5° C., the sugar content (by HPLC) and texture in each yogurt sample was assessed according to the Materials/Methods. The resulting sugar content is presented in FIG. 1 and the resulting apparent viscosity at shear rates of 11.7 Hz and 249 Hz is presented if FIGS. 2 and 3, respectively.

TABLE 1

| | GTF 0768 | vGTFJ |
|---|---|---|
| | (SEQ ID NO: 1) | (SEQ ID NO: 3) |
| Sample ID | (μL) | (μL) |
| 100:0% (GTF 0768:vGTFJ) | 37.5 | 0 |
| 90:10% (GTF 0768:vGTFJ) | 33.75 | 24.7 |
| 75:25% (GTF 0768:vGTFJ) | 28.125 | 61.75 |
| 50:50% (GTF 0768:vGTFJ) | 18.75 | 123.5 |
| 25:75% (GTF 0768:vGTFJ) | 9.375 | 185.25 |
| 10:90% (GTF 0768:vGTFJ) | 3.75 | 222.3 |
| 0:100% (GTF 0768:vGTFJ) | 0 | 247 |
| 2% starch | 0 | 0 |
| 3% starch | 0 | 0 |

It was found that all samples with one or both glucosyltransferase enzymes added had achieved at least 25% sugar reduction as compared to the reference samples containing starch (FIG. 1). The highest sugar reduction was found using GTF 0768 only and least when using vGTFJ only. This was due to the fact that GTF 0768, as compared to vGTFJ, used lactose as an acceptor molecule to a higher extent and released less free glucose byproduct (data not shown).

It was furthermore clear that treatment with vGTFJ alone (0:100% (GTF 0768: vGTFJ)) resulted in an increased apparent viscosity at 11.7 Hz and 249 Hz as compared to what was previously described in WO2020/010176 for another alpha-1,3-glucan-producing GTF, and that treatment with GTF 0768 alone (100:0% (GTF 0768: vGTFJ)) resulted in low apparent viscosity (FIGS. 2 and 3). It was furthermore found (FIGS. 2 and 3) that some of the enzyme combinations only resulted in a slight increase in apparent viscosity (10:90% (GTF 0768: vGTFJ), 25:75% (GTF 0768: vGTFJ) and 50:50% (GTF 0768: vGTFJ)) as compared to dosing the GTF 0768 enzyme alone (100:0% (GTF 0768: vGTFJ)). Surprisingly, the apparent viscosity of the 90:10% (GTF 0768: vGTFJ) samples at both 11.7 Hz and 249 Hz exceeded the apparent viscosity obtained when using vGTFJ alone; this was especially the case for the perception of mouthfeel at shear rate 249 Hz (FIG. 3). The 90:10% (GTF 0768: vGTFJ) addition had a significant impact on texture and it was the only GTF-treated sample to exceed the apparent viscosity obtained when using 3% starch (FIGS. 2 and 3). Thus, the texture of the yogurt product could be tailored by merely adjusting the dose relation of GTF 0768 and vGTFJ.

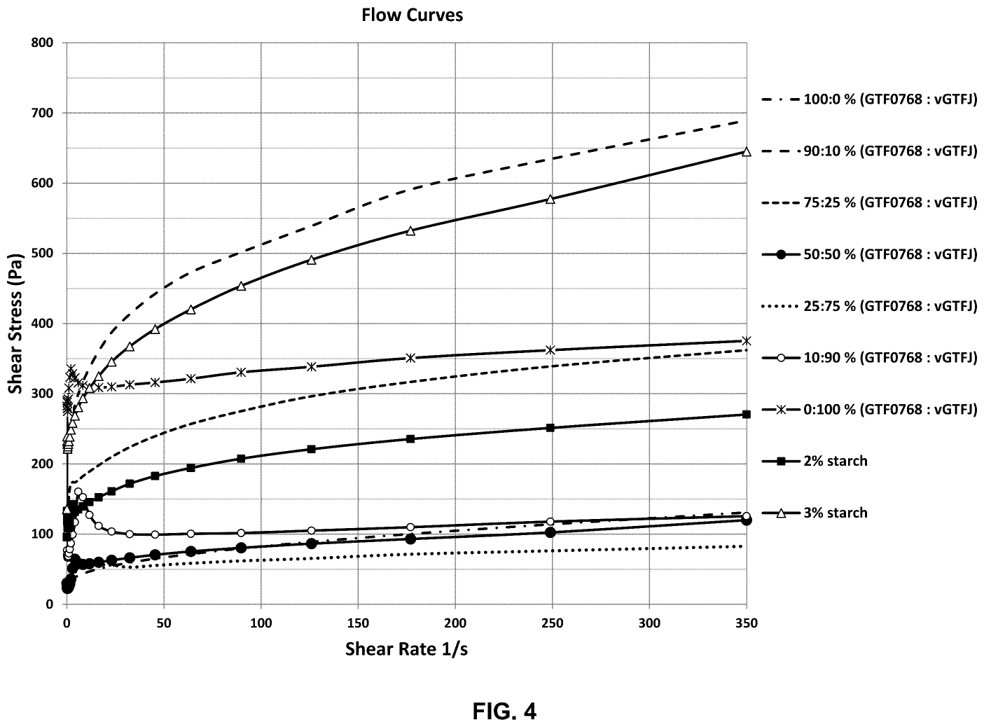

Since vGTFJ (SEQ ID NO:3) forms insoluble alpha-1,3-glucan and GTF 0768 (SEQ ID NO:1) forms a soluble dextran, it was hypothesized that a specific relation of these two types of enzymes results in formation of a soluble dextran-alpha-1,3-glucan graft copolymer that could provide a gel-like texture. This hypothesis was supported by the flow curves presented in FIG. 4. The samples with insoluble alpha-1,3-glucan had an initial shear thinning effect at a shear rate below 40 Hz and then increased slightly in shear stress with an increasing shear rate. In contrast, samples with the soluble dextran and samples with dextran-alpha-1,3-glucan graft copolymer had a shear thickening effect very similar to starch.

### Example 2

### In Situ-Produced Dextran-Alpha-1,3-Glucan Graft Copolymers in Yogurt Fermented with Various Cultures

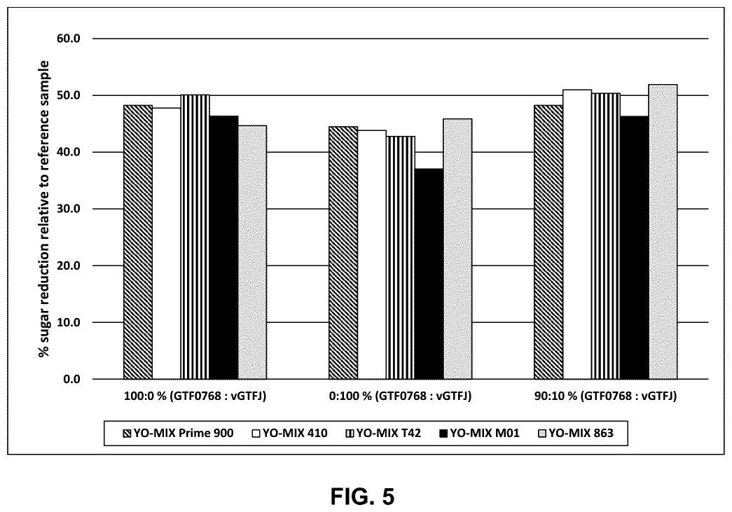

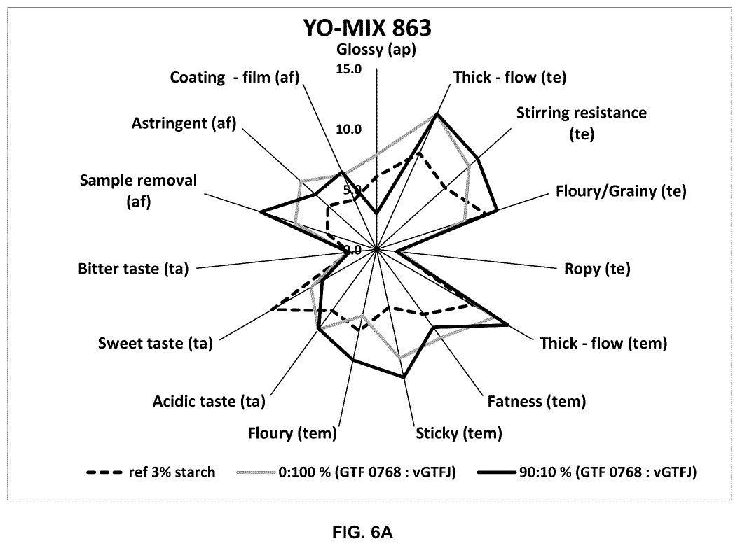

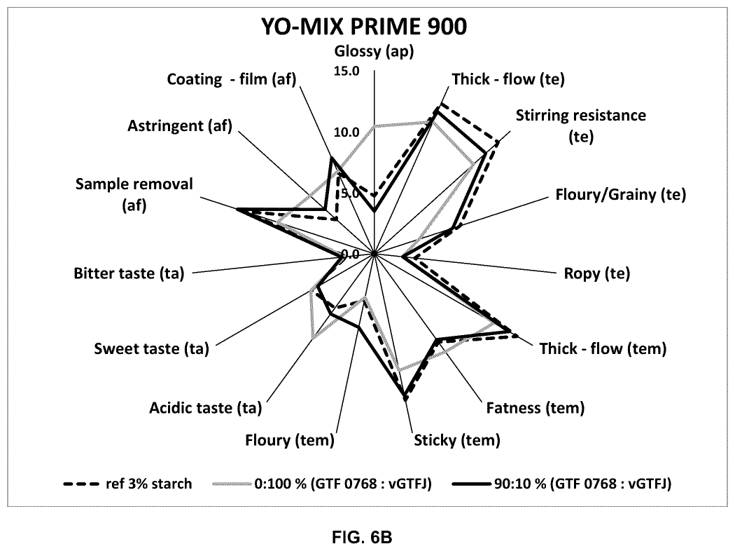

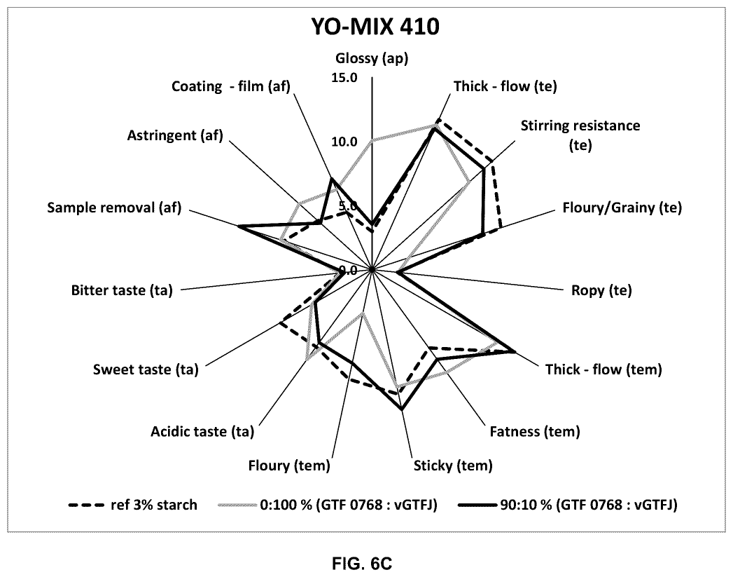

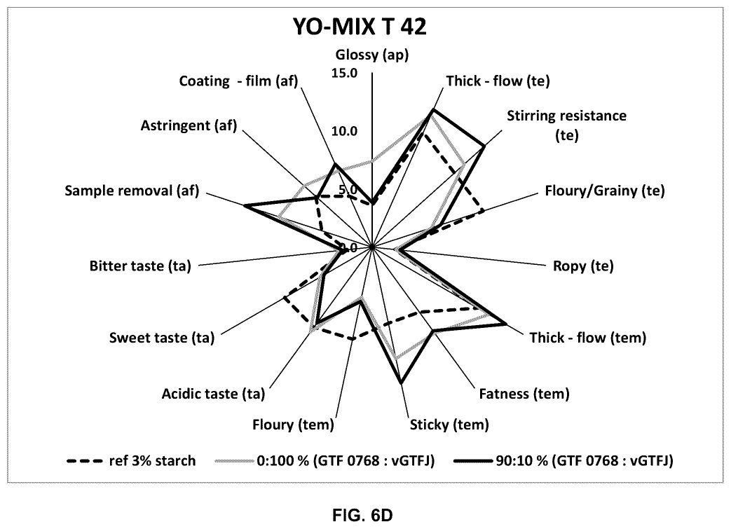

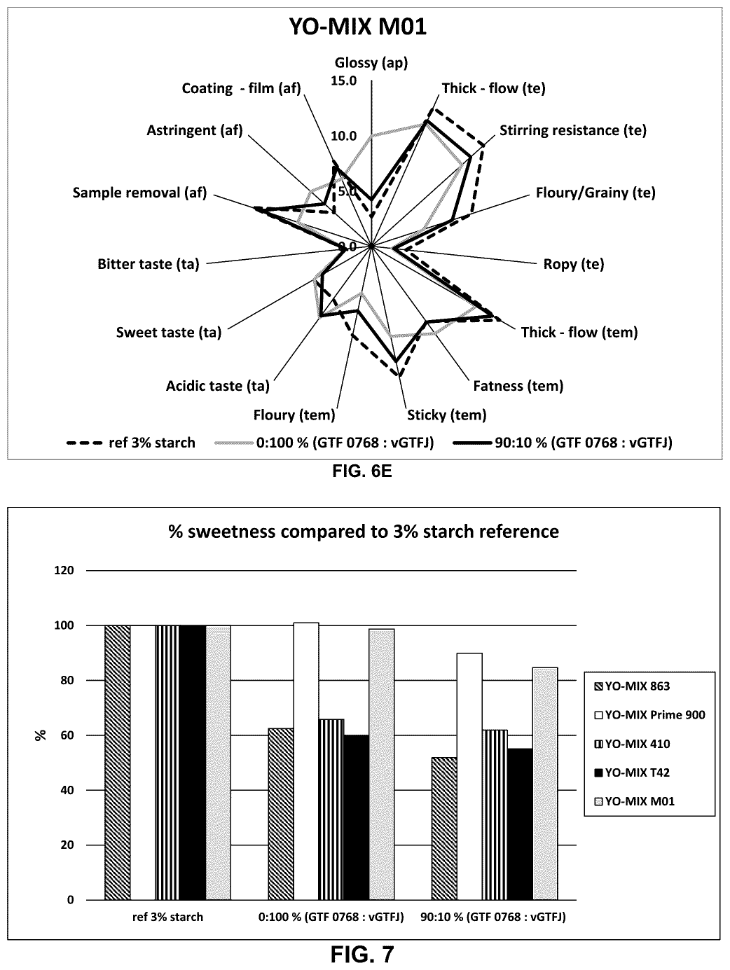

The sugar reduction and texturing effects of GTF 0768 (SEQ ID NO:1) and vGTFJ (SEQ ID NO:3), alone or in combination, were investigated in a yogurt production set-up (scale: 60 g). Fresh milk was standardized to 4.0% (w/w) protein, 1.0% (w/w) fat, 8.0% (w/w) sucrose and 3% starch for the reference samples, which were homogenized and pasteurized as described in the Materials/Methods. GTF enzymes were added at the dosage level shown in Table 2, where 100% GTF 0768 or vGTFJ was the amount of enzyme sample needed to fully convert the sucrose within the time of fermentation. YO-MIX 863, YO-MIX PRIME 900, YO-MIX M01 and YO-MIX T42 were used as starter cultures (available from IFF). After 14 days of storage at 5° C., the sugar content of each thus prepared yogurt sample was quantified according to HPLC (Materials/Methods), and sweetness and texturing effect were assessed by sensory evaluation. The sensory evaluation was performed with a trained panel of ten individuals. Three training sessions and three analysis sessions were done with sample servings of 30 mL at 10° C. For mouth rinsing between samples, rose hips tea and crackers were used. The relative sugar content in each sample is presented in FIG. 5. The sensory data are presented in spider plots in FIGS. 6A-E, and the sweetness relative to the starch references is shown in FIG. 7.

TABLE 2
Enzyme Dosages

| | GTF 0768 | vGTFJ |
|---|---|---|
| Sample ID | (SEQ ID NO: 1) | (SEQ ID NO: 3) |
| 100:0% (GTF 0768:vGTFJ) | 100% | 0 |
| 0:100% (GTF 0768:vGTFJ) | 0 | 100% |
| 90:10% (GTF 0768:vGTFJ) | 90% | 10% |
| 3% starch | 0 | 0 |

It was found that all samples with glucosyltransferase enzymes added had achieved at least 35% sugar reduction and up to 52% sugar reduction as compared to the reference samples containing starch.

As in Example 1, treatment of yogurt with GTF 0768 alone (100:0% [GTF 0768: vGTFJ]) had a low impact on texture and is therefore not shown in the spider plots of FIGS. 6A-E, whereas treatments with vGTFJ alone (0:100% [GTF 0768: vGTFJ]) or both vGTFJ and GTF 0768 (90:10% [GTF 0768: vGTFJ]) both had a significant increase in yogurt texture. The treatment with vGTFJ alone also provided yogurt that was more smooth/glossy and less grainy/floury as compared to reference yogurt with starch. It was surprisingly found that more than 80% of the sweetness (relative to starch reference) was maintained in yogurt having mild (pH 4.6) cultures (YO-MIX PRIME 900 and YO-MIX M01), which was in contrast to what was observed with the yogurts having more acidic (pH 4.3) cultures (YO-MIX 863, YO-MIX 410, YO-MIX T42) (FIG. 7), even though all the sucrose had been converted in each enzyme-treated yogurt.

### Example 3

### Combined Addition of Glucosyltransferase and Beta-Galactosidase in Yogurt Production

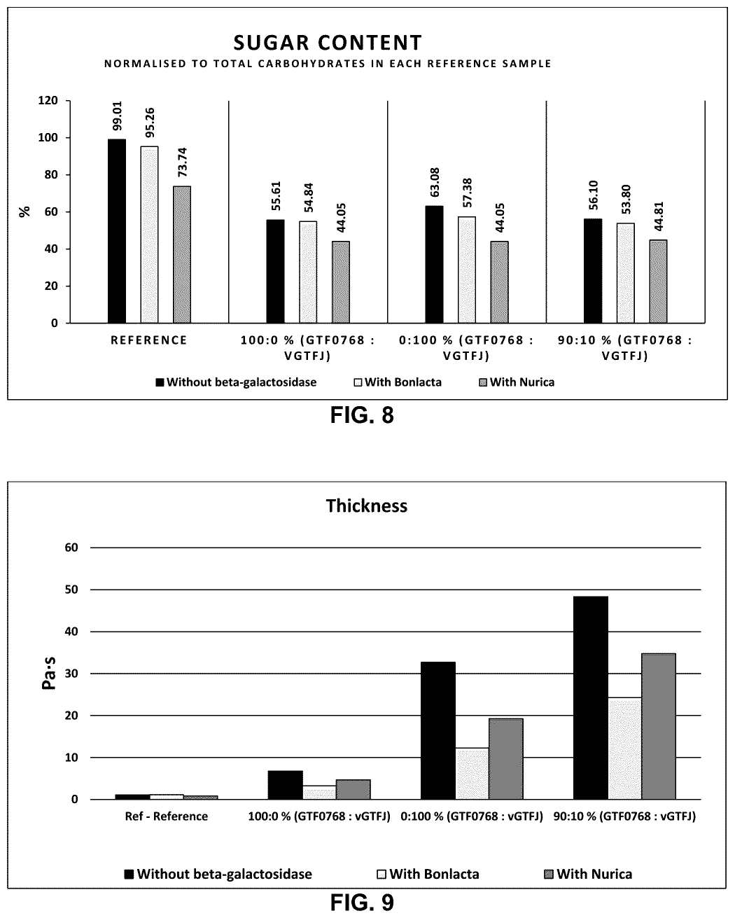

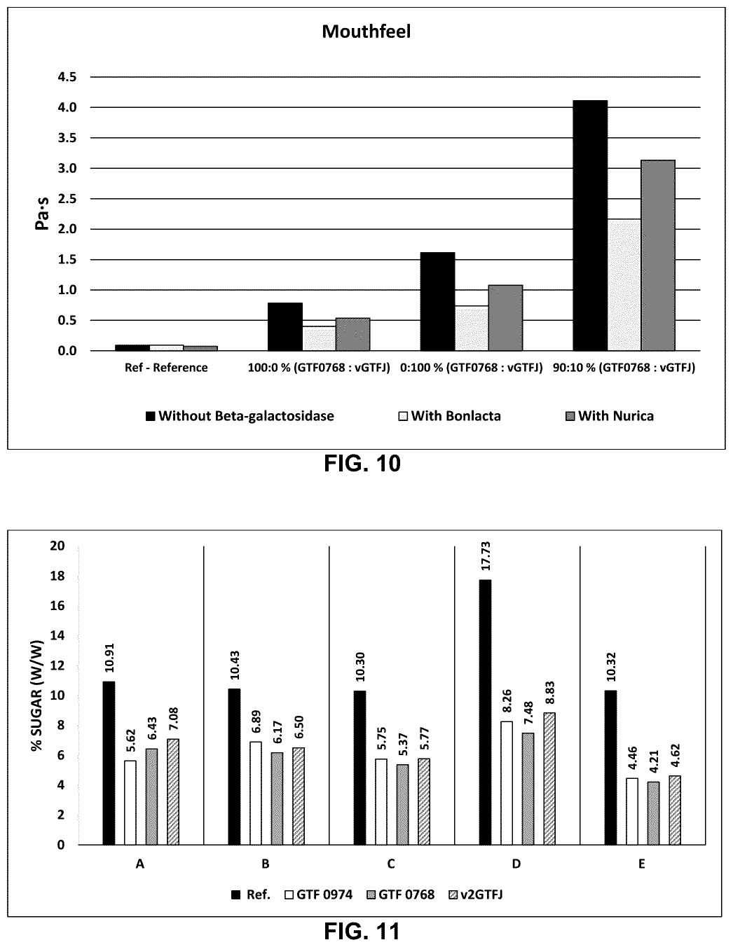

The sugar reduction and texturing effects of GTF 0768 (SEQ ID NO:1) and vGTFJ (SEQ ID NO:3), alone or in combination, were investigated in joint addition with beta-galactosidase having either lactase or both lactase and transgalactosylation activity in a yogurt production set-up (scale: 60 g). The individual dosages of the GTF 0768 and vGTFJ enzymes were normalized in which 100% dosage was the dosage necessary to provide full sucrose conversion when added at the inoculation step. Bonlacta™ (IFF) lactase (i.e., a type of beta-galactosidase) was dosed at 0.9% (v/w) and Nurica™ (IFF), a lactase with transgalactosylation activity (produces galacto-oligosaccharides [GOS]) was dosed at 0.21% (v/w). Fresh milk was standardized to 4.0% (w/w) protein, 1.0% (w/w) fat and 8.0% (w/w) sucrose, and homogenized and pasteurized as described in the Materials/Methods. All enzymes were added at the inoculation step per the dosages presented in Table 3. YO-MIX 410 was employed as the starter culture. After three days of storage at 5° C., the sugar content and texture of the yogurt samples were assessed according to the Materials/Methods. The resulting sugar content is presented in FIG. 8 as values relative to the total quantified carbohydrates of each reference sample. The resulting apparent viscosities at shear rates of 11.7 Hz and 249 Hz are presented in FIGS. 9 and 10, respectively.

TABLE 3
Enzyme dosages

| | | | GTF 0768 | vGTFJ |
|---|---|---|---|---|
| | BONLACTA | NURICA | (SEQ ID NO: 1) | (SEQ ID NO: 3) |
| Sample ID | (HL) | (PL) | (μL) | (μL) |
| Ref - Reference | 0 | 0 | 0 | 0 |
| Ref - 100:0% (GTF | 0 | 0 | 32 | 0 |
| 0768:vGTFJ) | | | | |
| Ref - 0:100% (GTF | 0 | 0 | 0 | 210 |
| 0768:vGTFJ) | | | | |
| Ref - 90:10% (GTF | 0 | 0 | 28 | 21 |
| 0768:vGTFJ) | | | | |
| Bon - Reference | 54 | 0 | 0 | 0 |
| Bon - 100:0% (GTF | 54 | 0 | 32 | 0 |
| 0768:vGTFJ) | | | | |
| Bon - 0:100% (GTF | 54 | 0 | 0 | 210 |
| 0768:vGTFJ) | | | | |
| Bon - 90:10% (GTF | 54 | 0 | 28 | 21 |
| 0768:vGTFJ) | | | | |
| Nur - Reference | 0 | 128 | 0 | 0 |
| Nur - 100:0% (GTF | 0 | 128 | 32 | 0 |
| 0768:vGTFJ) | | | | |
| Nur - 0:100% (GTF | 0 | 128 | 0 | 210 |
| 0768:vGTFJ) | | | | |
| Nur - 90:10% (GTF | 0 | 128 | 28 | 21 |
| 0768:vGTFJ) | | | | |

In the reference sample with BONLACTA only, the lactose was fully hydrolyzed (data not shown) and only a small amount of DP3 oligomers was formed, hence about 95% of the carbohydrates in the sample was still considered sugar (i.e., DP1 [glucose, galactose] and DP2 [sucrose and lactose) (FIG. 8). The reference sample with NURICA only had 73.7% of the carbohydrates still considered as sugar as the NURICA formed galacto-oligosaccharides and lactosucrose. Addition of GTF 0768 and/or vGTFJ resulted in 37-46% sugar reduction (resulting in 54-63% residual sugar) in the samples either treated without beta-galactosidase or with BONLACTA. For the samples treated with NURICA and GTF 0768 and/or vGTFJ, the observed sugar reduction reached 56% leaving only 44% of the carbohydrates as sugar in the samples (FIG. 8).

The use of a beta-galactosidase together with GTF 0768 and/or vGTFJ resulted in a lower apparent viscosity at both shear rates of 11.7 Hz and 249 Hz as compared to samples without beta-galactosidase (FIGS. 9 and 10). The apparent viscosity at both shear rates of 11.7 Hz and 249 Hz was, however, still significantly higher than the reference samples without added glucosyltransferase enzyme(s). Again, consistent with Example 1, samples with the 90:10% (GTF 0768: vGTFJ) addition had the highest perception in mouthfeel at an apparent viscosity of 249 Hz, regardless of whether they were used with a beta-galactosidase or not. Thus, it was possible to combine the usage of GTF 0768 and vGTFJ with a beta-galactosidase to obtain a significant increase in texture while either rendering the yogurt product lactose-free (with BONLACTA) or with further reduced sugar content (with NURICA).

### Example 4

### Texturizing Effect and Sugar Reduction in Various Neutral Substrates Using Glucosyltransferase Enzymes In Situ

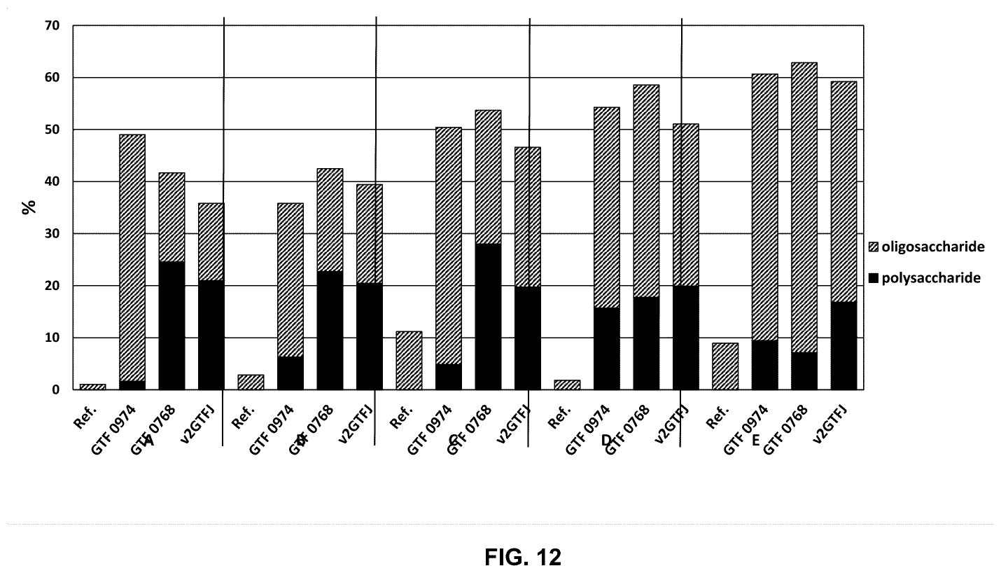

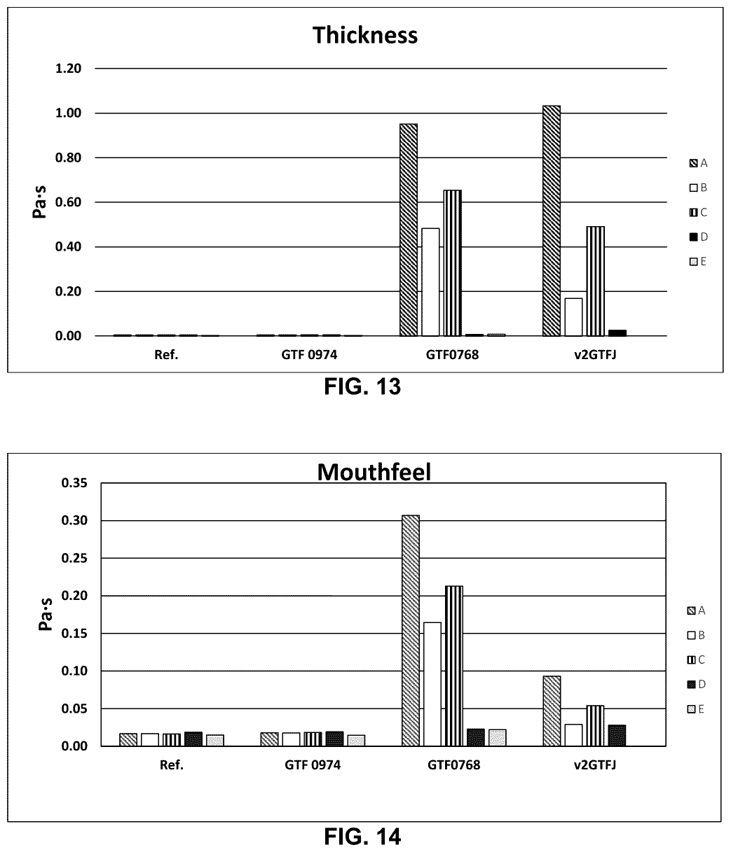

The texturizing effect of GTF 0768 (SEQ ID NO:1), GTF 0974 (SEQ ID NO:13), and a variant of S. salivarius GTF 6855 (“v2GTFJ”) in various milk preparations and buffered substrates was investigated. GTF 0974 (U.S. Pat. Appl. Publ. No. 2018/0291311, incorporated herein by reference) represents an S. salivarius glucosyltransferase-SI enzyme that produces alpha-1,3-glucan with about 93% alpha-1,3 linkages and about 7% alpha-1,6 linkages. v2GTFJ is an amino acid-modified S. salivarius glucosyltransferase enzyme that produces alpha-1,3-glucan with about 100% alpha-1,3 linkages at high yield (U.S. Pat. Appl. Publ. No. 2018/0072998, incorporated herein by reference). Substrates A-E were prepared as described in Table 4. GTF 0768, GTF 0974, or v2GTFJ were then added to each substrate and incubated for 24 hours at 5° C. to fully convert the sucrose in each preparation, after which the enzymes were heat-inactivated (10 min, 95° C.). After three days of storage at 5° C., the sugar content and texture of each preparation were assessed according to the Materials/Methods. The absolute sugar content in each preparation is presented in FIG. 11, and the percent total sugar reduction relative to each respective reference preparation (as initially prepared—no GTF added, and prior to incubations with other enzymes per Table 4) is presented in FIG. 12. FIG. 12 also shows the relative oligosaccharide (DP3+) and polysaccharide content of each preparation. The resulting apparent viscosities at shear rates of 11.7 Hz and 249 Hz are presented in FIGS. 13 and 14, respectively.

TABLE 4
Preparation of Substrates A-E

| Substrate | Preparation procedure |
|---|---|
| A | Sucrose was dissolved in MINI MILK (4.8% lactose) (Arla Foods, |
| | Denmark) to a final concentration of 7.5% (w/w). |
| B | 1.7 g/L BONLACTA was added to MINI MILK (4.8% lactose) and the |
| | milk was incubated at 5° C. for 24 hours. The enzyme was then |
| | inactivated by heating the milk to 95° C. for 10 min. The milk was cooled |
| | before adding sucrose to a final concentration of 7.5% (w/w). |
| C | 3.2 g/L NURICA was added to MINI MILK (4.8% lactose) and the milk |
| | was incubated at 5° C. for 24 hours. The enzyme was then inactivated |
| | by heating the milk to 95° C. for 10 min. The milk was cooled before |
| | adding sucrose to a final concentration of 7.5% (w/w). |
| D | Maltose was dissolved in MINI MILK (4.8% lactose) to a final |
| | concentration of 5% (w/w) before adding sucrose to a final |
| | concentration of 7.5% (w/w). |
| E | Maltose was dissolved in 0.05 M sodium phosphate buffer (pH 5.5) to a |
| | concentration of 5% (w/w). FoodPro ® TGO enzyme was then added to |
| | 0.125 g/L and incubated at 60° C. for 1 hour. The enzyme was then |
| | inactivated by heating to 95° C. for 10 min. The sample was cooled to |
| | 5° C. before adding sucrose to a final concentration of 7.5% (w/w). |

It was found that a >35% sugar reduction could be achieved in all substrates treated with a GTF enzyme (FIG. 11). The highest level of sugar reduction (>50%) observed in GTF-treated substrates was with substrates D and E, which initially contained maltose (substrate D) or isomalto-oligosaccharides (IMO) (substrate E, possibly also some residual maltose). It is noted that substrate E, prior to GTF treatment, contained IMO by virtue of having been treated with the FoodPro® TGO enzyme, which is a transglucosidase that converts maltose into IMO (mostly DP3-DP5) (mainly by transferring glucose from maltose to another maltose or already-extended maltose). Based on these data, it is clear that each GTF enzyme added to substrates D and E used maltose and/or IMO as acceptor molecules for oligo- and polysaccharide synthesis. In comparing substrates A-C with substrates D-E, GTF 0974 caused higher polysaccharide formation in substrates A-C, while GTF 0768 and v2GTFJ caused higher oligosaccharide formation in substrates D-E (FIG. 12).

Surprisingly, only the addition of GTF 0768 or v2GTFJ resulted in increases of apparent viscosity at both shear rates of 11.7 Hz and 249 Hz in substrate A-C (FIGS. 13-14). In contrast to the fresh fermented yogurt samples (see above), GTF 0768 had the highest impact on apparent viscosity at the 249 Hz shear rate, which was most probably due to the fact that it produces soluble polysaccharide. The change in characteristics of reaction products to mainly oligosaccharides in substrates D and E resulted in samples with low or no increase in texture. It was therefore clear that, by adding a GTF enzyme such as GTF 0768 or v2GTFJ, a high sugar reduction (>50%) could be obtained in food products having both sucrose and maltose (and/or IMO) without significantly changing the texture.

### Example 5

### In Situ-Produced Dextran-Alpha-1,3-Glucan Graft Copolymers in Neutral Beverages

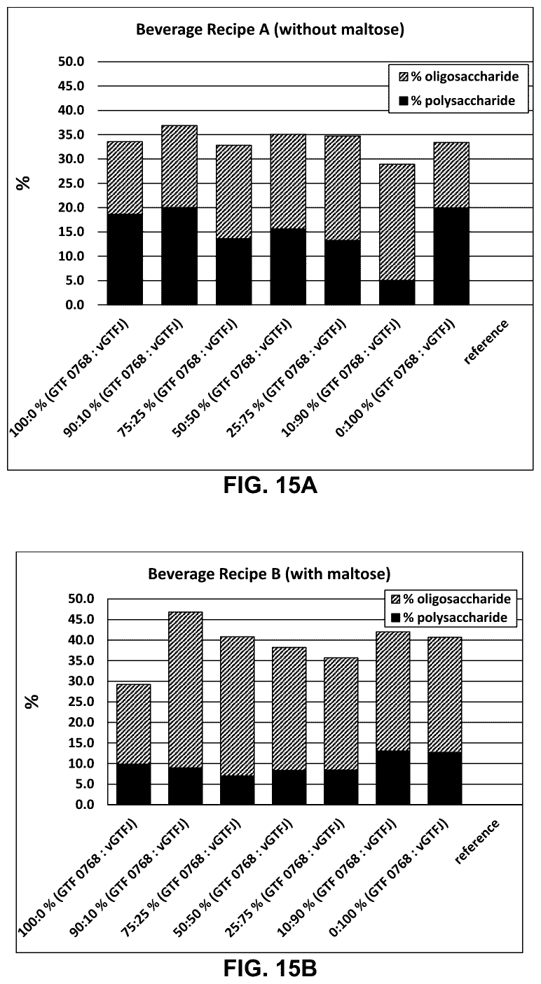

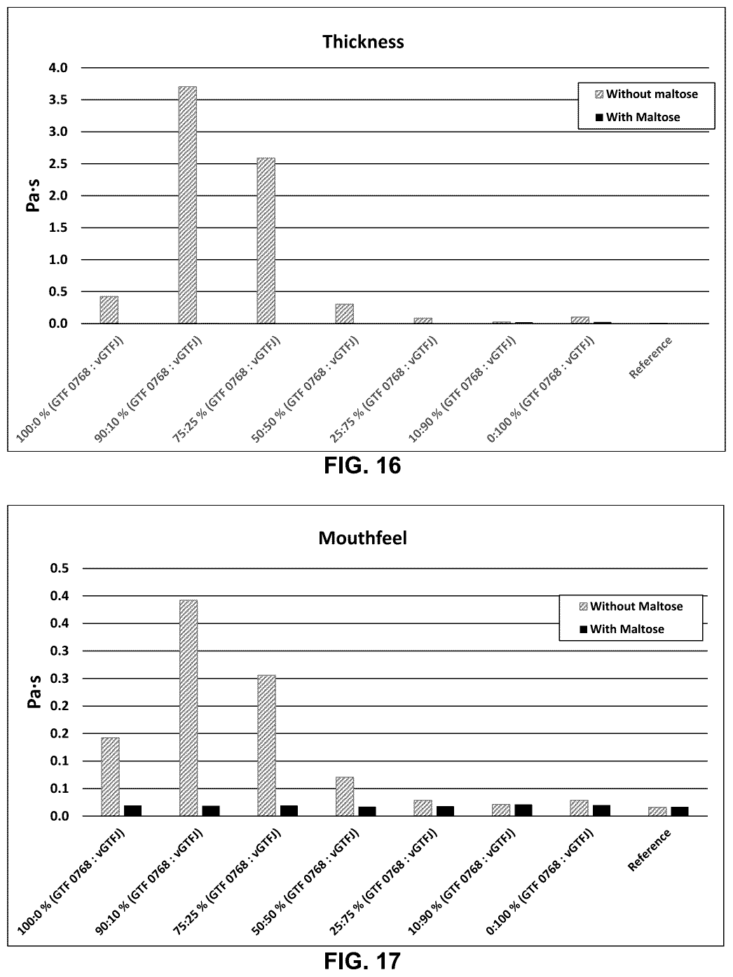

The texturizing effect of GTF 0768 (SEQ ID NO:1) and vGTFJ (SEQ ID NO:3) added alone or in combination was investigated in two neutral beverage recipes. The beverage base was 10% (w/w) skimmed milk powder and 5% (w/w) sucrose with (B) or without (A) 4% (w/w) maltose dissolved in tap water. The GTF enzymes were added to the cold beverage base as presented in Table 5 and incubated 24 hours at 5° C. before heat-inactivating the GTF enzymes at 95° C. for 10 minutes. The individual enzyme dosages of the GTF enzymes were normalized at which 100% dosage is the dosage necessary to provide full sucrose conversion during the 24 hours at 5° C. After three days of storage at 5° C., the sugar content and texture of each preparation were assessed according to the Materials/Methods. FIGS. 15A (without maltose) and 15B (with maltose) show the percent total sugar reduction relative to the reference preparation. FIGS. 15A-B also show the relative oligosaccharide (DP3+) and polysaccharide content of each preparation. The resulting apparent viscosities at shear rates of 11.7 Hz and 249 Hz are presented in FIGS. 16 and 17, respectively.

TABLE 5
Enzyme Dosages

| | GTF 0768 | vGTFJ |
|---|---|---|
| Sample ID | (SEQ ID NO: 1) | (SEQ ID NO: 3) |
| 100:0% (GTF 0768:vGTFJ) | 100% | 0% |
| 90:10% (GTF 0768:vGTFJ) | 90% | 10% |
| 75:25% (GTF 0768:vGTFJ) | 75% | 25% |
| 50:50% (GTF 0768:vGTFJ) | 50% | 50% |
| 25:75% (GTF 0768:vGTFJ) | 25% | 75% |
| 10:90% (GTF 0768:vGTFJ) | 10% | 90% |
| 0:100% (GTF 0768:vGTFJ) | 0% | 100% |
| reference | 0% | 0% |

Although vGTFJ had a high texturizing effect in the fresh fermented products (see above), it was found in this Example to have almost no impact on texture (FIGS. 16-17) (similar to v2GTFJ, see above) due to the production of insoluble polysaccharides. Surprisingly, it was found that the combined addition of GTF 0768 and vGTFJ, which resulted in formation of soluble dextran-alpha-1,3-glucan graft copolymer, had an impressive effect on both texture readouts of thickness and mouthfeel (FIGS. 16-17). Further, texture could be tailored by adjusting the blend ratio of GTF 0768 and vGTFJ in the neutral beverage without maltose. The use of GTF 0768 alone also increased texture; however, the polysaccharides formed were perceived to be very sticky/ropy, which typically would not be preferred for a smooth drinkable beverage. Consistent with the results of Example 4, it was generally possible, with GTF treatment, to obtain a higher percent sugar reduction in neutral beverages having maltose (as compared to neutral beverages not having maltose) without significantly altering the texture, which is due to a higher preference for oligosaccharides formation (compare FIGS. 15A and 15B).

### Example 6

### Thickening of Plant-Based Yogurt Using Glucosyltransferase Enzymes In Situ

vGTFJ (SEQ ID NO:3) or a combination of GTF 0768 (SEQ ID NO:1) and vGTFJ (90:10% [GTF 0768: vGTFJ]) were evaluated in coconut yogurt. Yogurt stabilized with starch was used as a reference, and was made with 6% sucrose, 30% coconut cream (iTi Tropicals, Lawrence Township, New Jersey, USA), 6% tapioca starch, and 58% water. The experimental yogurt base contained 6% sucrose, 30% coconut cream, and 64% tap water. The ingredients for each of the two bases were blended under high agitation before pasteurization, which was performed by high-temperature short-time pasteurization at 195° F. (90.56° C.) for 30 seconds. Homogenization was downstream from the heating step at a total pressure of 3000 psi in a two-stage homogenizer. After homogenization, each mixture was cooled to 110° F. (43.3° C.), after which fermentation was done with Danisco® VEGE 022 culture. Fermentation was considered complete when the yogurt pH reached 4.65 or lower. Two different yogurt samples were made by adding GTF enzyme(s) to the experimental base in the fermentation step: (i) vGTFJ alone (0:100% [GTF 0768: vGTFJ]) and (ii) a combination of GTF 0768 and vGTFJ (90:10% [GTF 0768: vGTFJ]). A third yogurt was prepared in which no GTF enzyme was added to the experimental base in the fermentation step. A yogurt was also made using the reference yogurt base (base with tapioca starch) without addition of a GTF enzyme.

It was clear that the addition of GTF enzyme(s) provided a color benefit to the yogurt products. The reference yogurt with starch had a yellowish color. However, the two experimental yogurts having one or both GTF enzymes did not exhibit a significant change in color (data not shown) as compared to the yogurt produced (white color) using the experimental base with no added GTF enzyme.

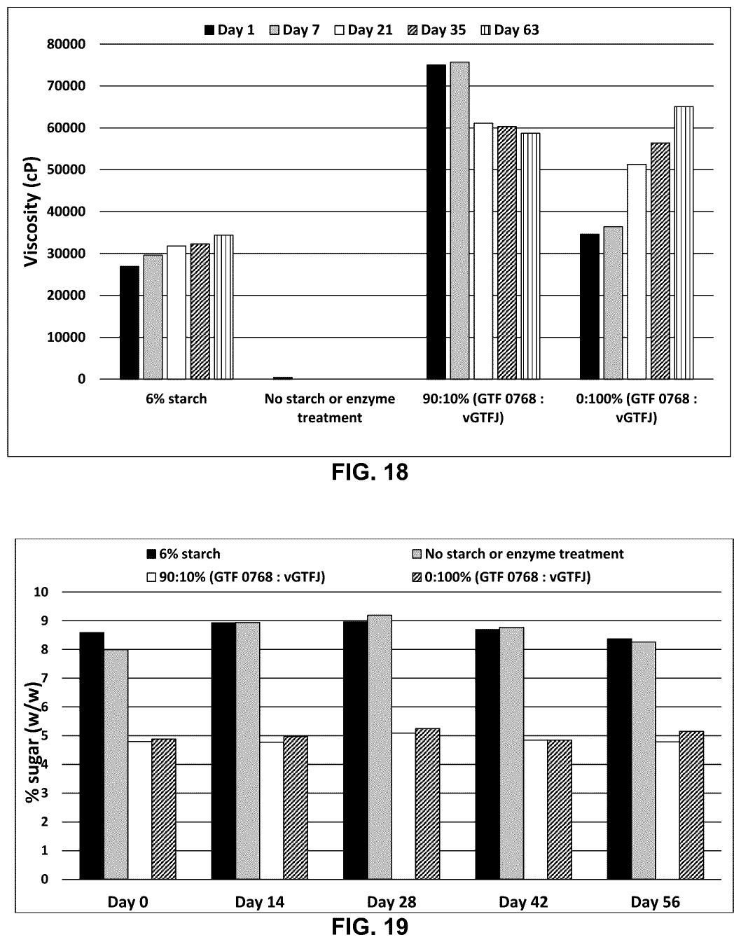

The viscosity of each yogurt was measured over a time period representing an example of product shelf life (63 days) (FIG. 18). Measurements were made 1, 7, 21, 35, or 63 days following yogurt production using a Brookfield DV3T™ Viscometer (Brookfield Engineering Laboratories, Middleboro, MA, USA) with RV spindle 6 after 30 s at a rotary speed of 20 rpm at 40° F. (4.4° C.). Yogurt pH values measured throughout this time period are presented in Table 6.

TABLE 6
Yogurt pH

| Sample ID | Day 1 | Day 21 | Day 35 | Day 65 |
|---|---|---|---|---|
| Reference (6% starch) | 4.48 | 4.50 | 4.49 | 4.46 |
| 90:10% (GTF 0768:vGTFJ) | 4.53 | 4.58 | 4.59 | 4.61 |
| 0:100% (GTF 0768:vGTFJ)) | 4.41 | 4.46 | 4.42 | 4.40 |

Yogurt sugar composition was also measured throughout this time period at days 0, 14, 28, 42 and 56 by HPLC (Materials/Methods). The % (w/w) total sugar of each sample is presented in FIG. 19.

The control yogurt without added GTF enzyme or starch had low viscosity throughout its shelf life (FIG. 18). In contrast, the reference yogurt with starch and both experimental yogurts with added GTF enzyme(s) had high viscosity. All the yogurts had a similar pH, from production to throughout the shelf life. In the samples containing added GTF enzyme(s), sugar was reduced by approximately 40%, which was consistent throughout the shelf life tested. It was furthermore found that almost no oligosaccharides formed (<0.6% w/w) in the GTF-treated yogurts, unlike what was observed in dairy-based yogurts above (e.g., Examples 4-5) that had initially contained endogenous or added disaccharides such as lactose or maltose. This lack of oligosaccharide production allowed for a higher proportion of polysaccharide formation, which in turn led to pronounced texture formation (thickening) (FIG. 18).

### Example 7

### Improved Physical Properties of Ice Cream Produced Using Glucosyltransferase Enzymes In Situ

Ice creams were prepared according to Table 7 with or without various combinations of glucose syrup, hydrocolloid, 0.32% NURICA, 0.17% BONLACTA, GTF 0768 (SEQ ID NO:1) and vGTFJ (SEQ ID NO:3). The ice cream base for samples 2102-3-(2-7) was prepared by mixing water, skimmed milk powder (9%), sucrose (12%), whey powder (2.5%) and emulsifier (0.35%, CREMODAN SUPER MB) at room temperature before adding refined coconut oil (4%, KRISTAL) and heating to 70° C. The ice cream bases were then homogenized at 78° C. and pasteurized at 84° C. for 30 seconds. After cooling to 5° C., the various enzymes were added per Table 7 during ice cream maturation, and the mixes were incubated 24 hours before freezing (light extrusion with 100% overrun) in a hardening operation at −30° C. The individual enzyme dosages of GTF 0768 and vGTFJ were normalized at which 100% dosage is the dosage necessary to provide full sucrose conversion during the 24 hours at 5° C. A reference ice cream (2021-3-1) was also prepared further containing glucose syrup (4%) and hydrocolloid (0.2%, CREMODAN DC 100).

The melting profile and texture attributes (hardness, cohesiveness, adhesiveness) of the ice cream samples were measured according to the Materials/Methods.

TABLE 7

| Sample ID | Sample description |
|---|---|
| 2102-3-1 | +hydrocolloid, +glucose syrup, no enzyme |
| 2102-3-2 | no enzyme |
| 2102-3-3 | 100:0% (GTF 0768:vGTFJ) |
| 2102-3-4 | 0:100% (GTF 0768:vGTFJ) |
| 2102-3-5 | 10:90% (GTF 0768:vGTFJ) |
| 2102-3-6 | 0:100% (GTF 0768:vGTFJ) and NURICA |
| 2102-3-7 | 0:100% (GTF 0768:vGTFJ) and BONLACTA |

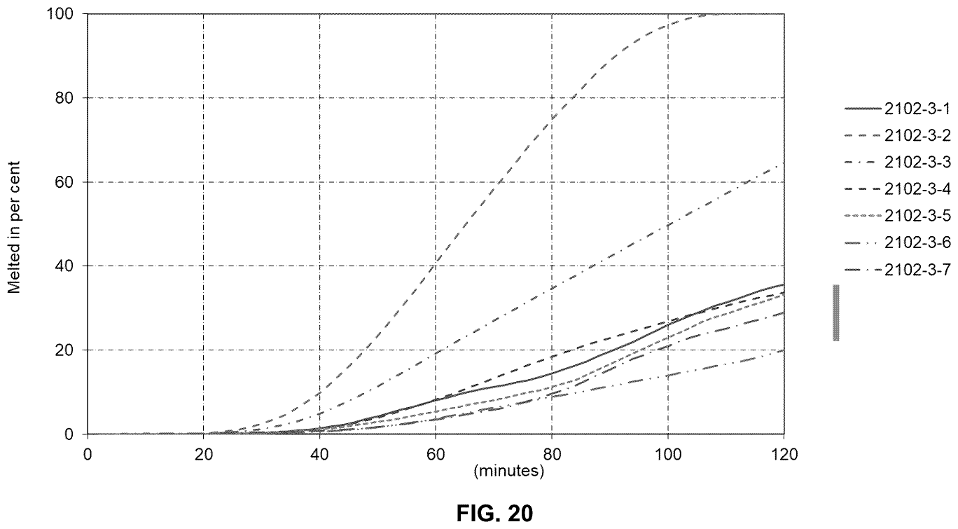

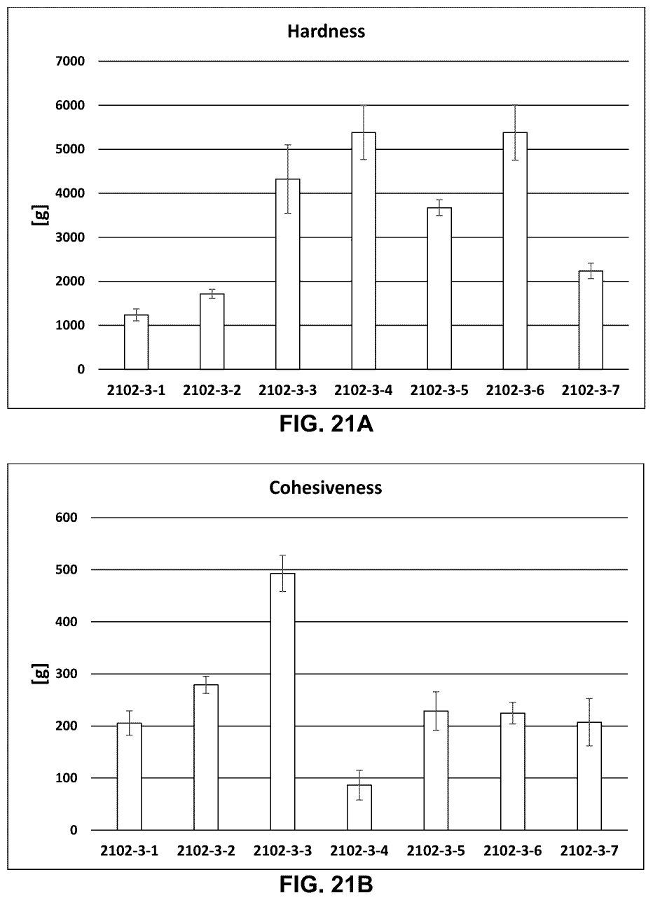

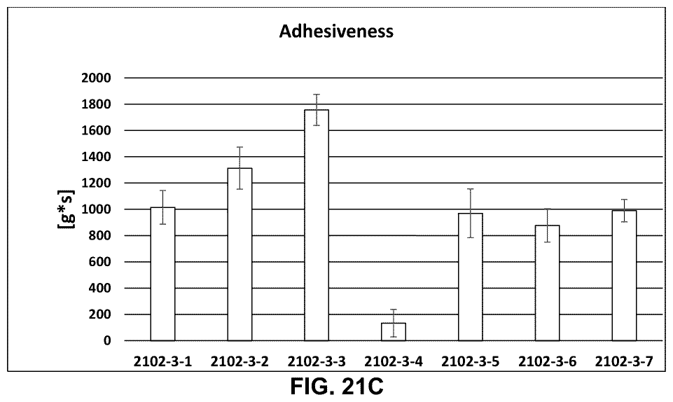

Samples 2102-3-(3-7) all had between 47-60% sugar reduction relative to sample 2102-3-2. Surprisingly, all samples with enzyme addition that included the vGTFJ enzyme had an improved melting profile (slower melting) relative to the plain ice cream base (sample 2102-3-2) (FIG. 20). This melting profile was at least on par, or better than, the melting profile of the reference sample containing hydrocolloid and glucose syrup (sample 2102-3-1). Furthermore, it was found that when the vGTFJ enzyme was combined with either GTF 0768 or a beta-galactosidase (NURICA or BONLACTA), the ice cream samples performed on par with the reference sample containing hydrocolloid and glucose syrup (sample 2102-3-1) on both cohesiveness and adhesiveness (FIGS. 21B and 21C). Only hardness increased in all cases using either GTF 0768 or vGTFJ, but it was clear that hardness could be improved (lowered) through combined addition of GTF 0768 and vGTFJ (2102-3-5 vs. 2102-3-3 and 2102-3-4) or by a combined addition of vGTFJ and BONLACTA (2102-3-7) (FIG. 21A).

### Example 8

### Improved Physical Properties of Sweetened Condensed Milk Produced Using Glucosyltransferase Enzymes In Situ

The in situ effect of glucosyltransferase enzymes was investigated in a condensed milk application.

The recipe of each condensed milk trial sample (trial nos. 1-8) is displayed in Table 8. For producing each sample, 800 g skim milk powder (Arta, Aarhus, Denmark), 1523 g sucrose (Nordic Sugar, Copenhagen, Denmark) and approximately 800 g water were blended at 50° C. until no lumps were visible. The mix was further stirred and cooled to 5° C. For trials 3-8, GTF 0768 (SEQ ID NO:1) and/or vGTFJ (SEQ ID NO:3) were added to individual mixes, which were then further stirred for 10 minutes and left for 24 hours at 5° C. until further processing. For GTF enzyme dosing, 100% GTF 0768 or vGTFJ was the amount of enzyme sample needed to fully convert the sucrose within the 24-hour, 5° C. incubation. Trial samples 1 and 2 acted as references/controls and contained no GTF enzyme, but were otherwise treated the same as trial samples 3-8. Next, anhydrase milk fat (AMF, 315 g per trial, Coreman, Goé, Belgium) and an emulsifier (7 g of RECODAN™ RS 100, not included in trials 2 and 3) were melted together at 70° C., after which 7 g lecithin (SOLEC SF-D, IFF, Copenhagen, Denmark) was added to the melts. The different melts and corresponding skim milk/sugar slurries were combined and mixed at 50° C. The blends were subsequently pasteurized at 90° C. for 3 minutes, homogenized (35 bar, 80° C.) and cooled to 30° C. while agitating vigorously. Except for trial 3, lactose seeds (1.75 g crystalline lactose, Variolac® 992, Arla, Viby, Denmark) were added during this mixing. All mixes were than cooled to 15° C. and stored for 18 hours in a tank to allow crystallization. Afterwards, all the condensed milk samples were filled in cups and cooled to 5° C.

TABLE 8
Sweetened Condensed Milk Recipes

| | Trial Sample Number | | | | | | | |
|---|---|---|---|---|---|---|---|---|
| | 1 | 2 | 3 | 4 | 5 | 6 | 7 | 8 |
| | Sample ID | | | | | | | |
| | | | (—)Stab | | | | | |
| | | | (—)Seed | | | | | |
| | | | 50:50% | 100:0% | 90:10% | 50:50% | 10:90% | 0:100% |
| | | Ref. | (GTF 0768: | (GTF 0768: | (GTF 0768: | (GTF 0768: | (GTF 0768: | (GTF 0768: |
| | Ref. | (—)Stab | vGTFJ) | vGTFJ) | vGTFJ) | vGTFJ) | vGTFJ) | vGTFJ) |
| Butter oil | 9%^{a} | 9% | 9% | 9% | 9% | 9% | 9% | 9% |
| (AMF) | | | | | | | | |
| SOLEC | 0.2% | 0.2% | 0.2% | 0.2% | 0.2% | 0.2% | 0.2% | 0.2% |
| SF-D | | | | | | | | |
| Skimmed | 22.85% | 22.85% | 22.85% | 22.85% | 22.85% | 22.85% | 22.85% | 22.85% |
| milk | | | | | | | | |
| powder | | | | | | | | |
| Sucrose | 43.5% | 43.5% | 43.5% | 43.5% | 43.5% | 43.5% | 43.5% | 43.5% |
| Lactose | 0.05% | 0.05% | — | 0.05% | 0.05% | 0.05% | 0.05% | 0.05% |
| (Variolac ® | | | | | | | | |
| 992 | | | | | | | | |
| BG100) | | | | | | | | |
| RECODAN | 0.2% | — | — | 0.2% | 0.2% | 0.2% | 0.2% | 0.2% |
| RS 100 | | | | | | | | |
| Water | 24.2% | 24.4% | 22.83% | 23.80% | 23.55% | 22.58% | 21.60% | 21.36% |

^{a}All ingredient percent values are in wt %.

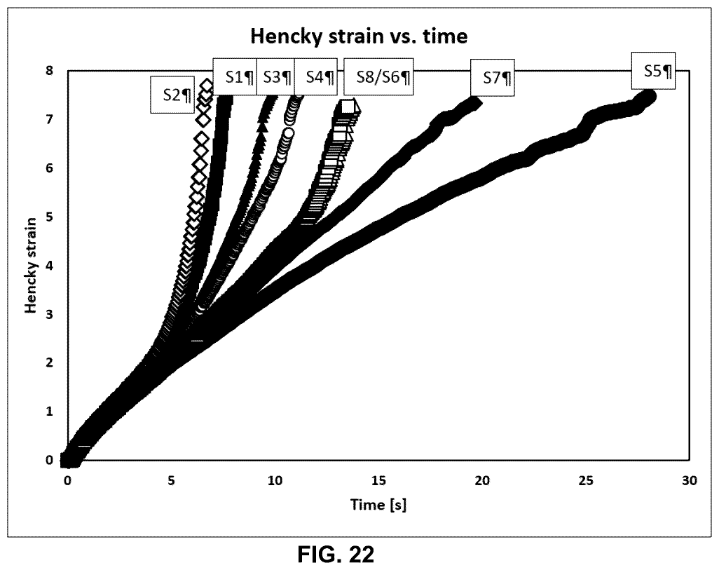

Extensional rheometry was performed on trial samples 1-8 (S1-S8, FIG. 22) using a VADER 1000 filament stretching device (Rheo Filament ApS, Copenhagen, Denmark). This device measures the dimensional change of, and the stresses acting on, a fluid bridge (filament) that forms between two plates during vertical separation of the plates. In this particular experiment, a constant vertical separation velocity of 1 mm/s was used. From the change of filament radius, a so-called Hencky strain is calculated. The degree of vertical separation that a filament can sustain (i.e., how “stringy” the sample is) in mm is numerically the same as the time the filament is stable, which follows from the use of a 1 mm/s separation velocity.

FIG. 22 shows Hencky strain vs. time for trial samples 1-8 (S1-S8), across all the samples, significant differences in strain values were observed at times greater than 5 seconds. From this point onward the samples varied significantly in their stretchability, or “stringiness”, as can be seen by the increasing time a filament can be sustained under deformation. Based on the data in FIG. 22, the samples can be ranked in terms of their stringiness/stretchability as follows: S5>S7>S8≈S6>S4>S3>S1>S2. It is notable that, while treatments with either GTF 0768 (S4) or vGTFJ (S8) alone, or with a 50%:50% combination of both GTF enzymes (S6), increased stringiness/stretchability as compared to the corresponding reference/control sample that did not receive any GTF treatment (S1), treatments with a 90% GTF 0768: 10% vGTFJ combination (S5) or a 10% GTF 0768: 90% vGTFJ combination (S7) resulted in greater increases in stringiness/stretchability (FIG. 22).

When the above analysis was performed at 50 mm/s, filament lengths of 40 mm to 65 mm at break point were observed (data not shown).

### Example 9

### Controlled Texture Formation in Plant-Based Compositions Using Glucosyltransferase Enzymes In Situ

Five 102.6-g sample solutions (Samples 1-5) were prepared, each comprised of tap water and sucrose (40 wt %). The sample solutions were brought to 35° C. and a high texturizing blend of GTF 0768 (SEQ ID NO:1) and vGTFJ (SEQ ID NO:3) was added to four of the solutions (Samples 2-5) at time zero. At timepoints specific for each sample, strawberry purée (30 g), strawberry pieces (70 g) and sucrose (6.4 g) were added to Samples 2 (15 minutes), 3 (20 minutes), 4 (25 minutes), and 5 (30 minutes). These same ingredients/amounts were also added to the reference sample without GTF enzymes (Sample 1) at the 30-minute timepoint. Following this addition, all of Samples 1-5 were incubated for a total time of 60 minutes (e.g., Sample 4 was incubated for 35 minutes after the addition) at 35° C. and then heat-treated for 5 minutes at 95° C. The sugar composition of each final sample product was measured by HPLC (Materials/Methods) and the samples were subjected to visual/manual evaluation.

The results of the visual/manual evaluation are presented in Table 9. Notably, based on these data, it was evident that texture/viscosity resulting from GTF enzyme activity could be modified by varying the time at which fruit ingredients were added. While early addition of fruit ingredients reduced the texturization effects of GTF activity in the final product (e.g., Sample 2, Table 9), later addition allowed GTF activity that was sufficient to provide texturization to the final product (e.g., Samples 4 and 5, Table 9). Likely consistent with these visual data, early addition of fruit ingredients resulted in a higher ratio of oligosaccharide-to-polysaccharide formation, whereas later addition of fruit ingredients resulted in lower ratio of oligosaccharide-to-polysaccharide formation (data not shown).

TABLE 9
Effect of Fruit Ingredient Addition on GTF-Based Texturization of a Food Product

| Sample | Time without | Total incubation | |
|---|---|---|---|
| No. | fruit ingredients | time at 35° C. | Visual evaluation of sample products |
| 1 | 30 min | 60 min | Thin, liquid texture. Fruit pieces |
| no GTFs | | | floating. |
| 2 | 15 min | 60 min | Thin, liquid texture, but more texture |
| | | | than Sample 1. Fruit pieces floating. |
| 3 | 20 min | 60 min | Still liquid texture, but more texture |
| | | | than Sample 2. Fruit pieces |
| | | | distributed. |
| 4 | 25 min | 60 min | Soft gel with good marmelade texture. |
| | | | Fruit pieces evenly distributed. |
| 5 | 30 min | 60 min | Strong gel. Fruit pieces evenly |
| | | | distributed. More force required to mix |
| | | | to yield product without gel pieces. |

### Example 10

### Glucosylation of Steviol Glycosides Rebaudioside a and Stevioside Using Glucosyltransferase Enzymes

Aqueous solutions were prepared having 20 mM sucrose, 100 mM sodium phosphate buffer (pH 6.5) and 1 mM of an individual Stevia component: rebaudioside A (Reb-A 99, PureCircle), rebaudioside D (PCS-3001 Reb. D, PureCircle), rebaudioside M (PCS-3018 Bio REB M 90, PureCircle), or stevioside (PCS-2005 Steviosid, PureCircle). Each solution was aliquoted in 10 mL portions and heated to 35° C. At time zero, GTF 6855 (SEQ ID NO:5 but starting with a valine instead of a methionine, 3.3 mg/L), vGTFJ (SEQ ID NO:3, 3 mg/L), GTF 0768 (SEQ ID NO:1, 1 mg/mL), or GTF 0974 (SEQ ID NO:13, 3.3 mg/mL) were individually added to the aliquots (for a total of 16 test samples) (final enzyme concentrations listed). Each sample was then incubated at 35° C. An aliquot from each sample was removed after 0, 15, 30, 60, 120 and 180 minutes and immediately inactivated by heating for 10 minutes at 95° C. (resulting in 48 samples in total). All the samples were stored frozen until analysed by ultra-high performance liquid chromatography (UHPLC, Materials/Methods).

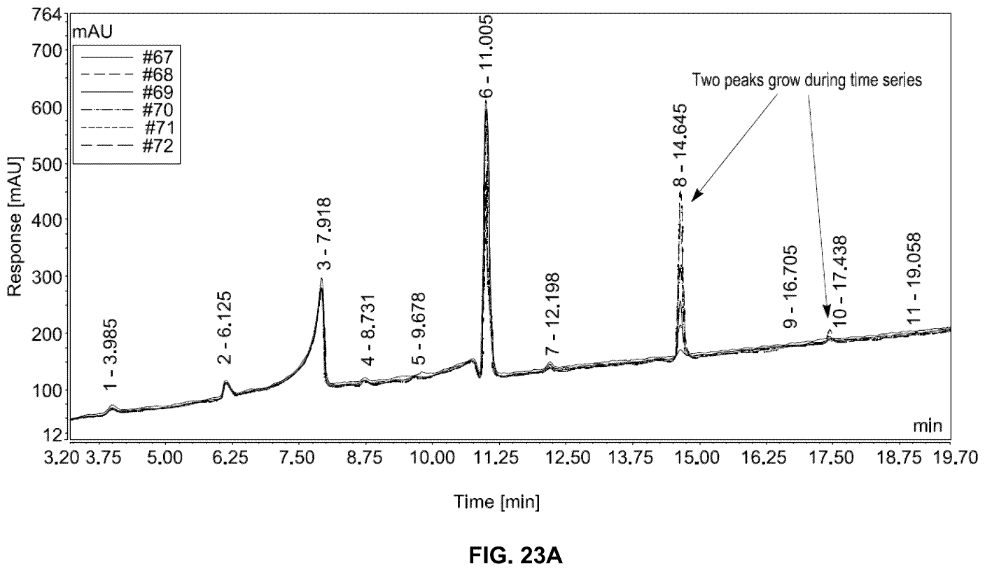

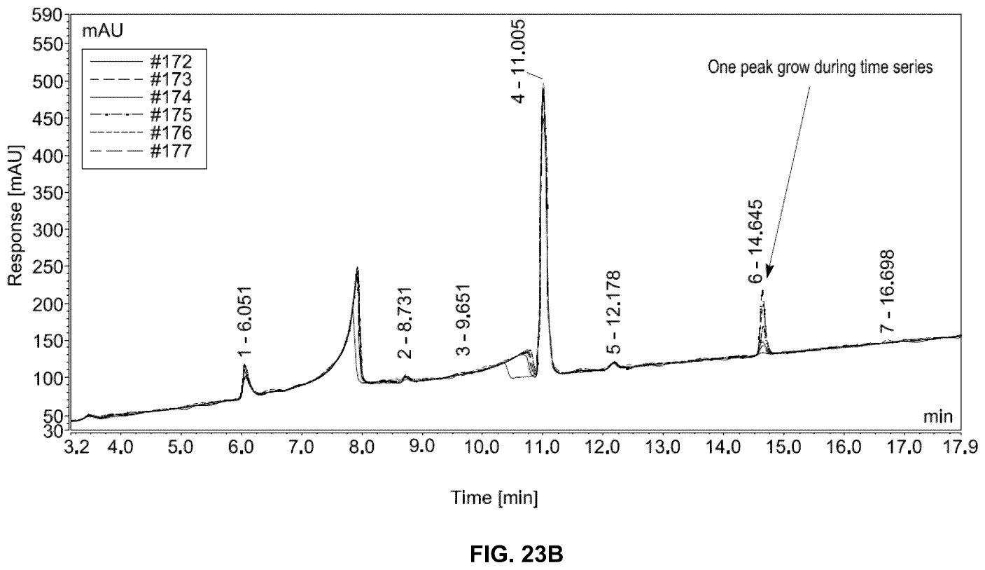

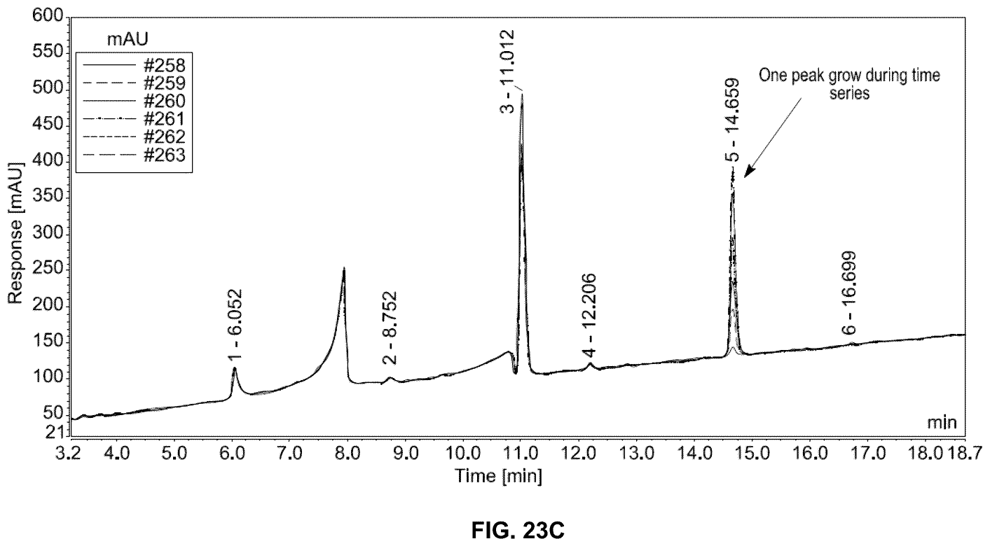

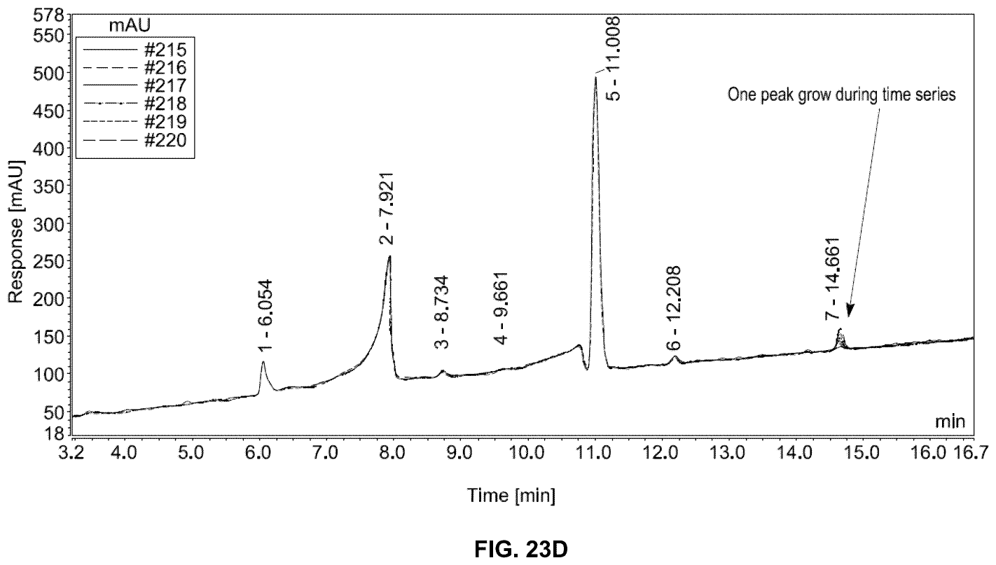

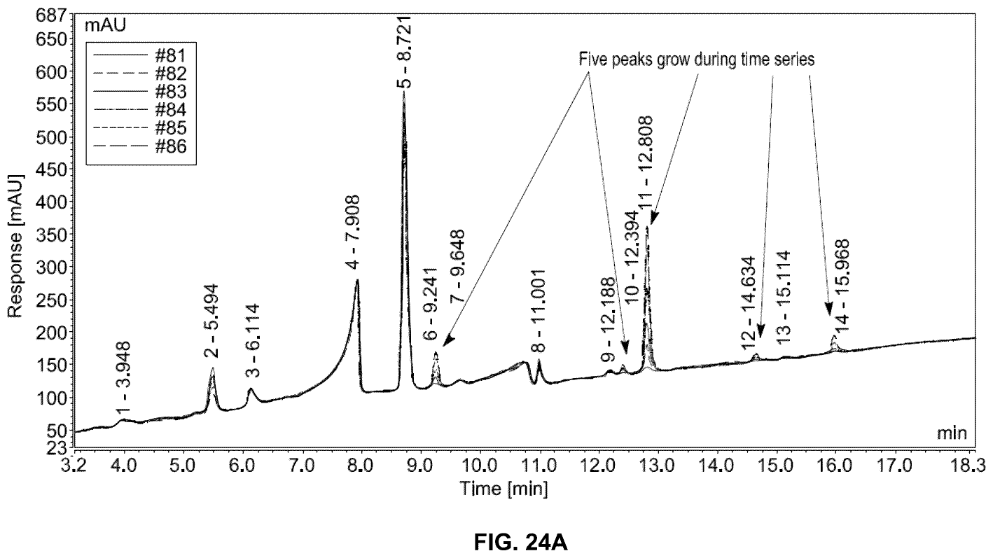

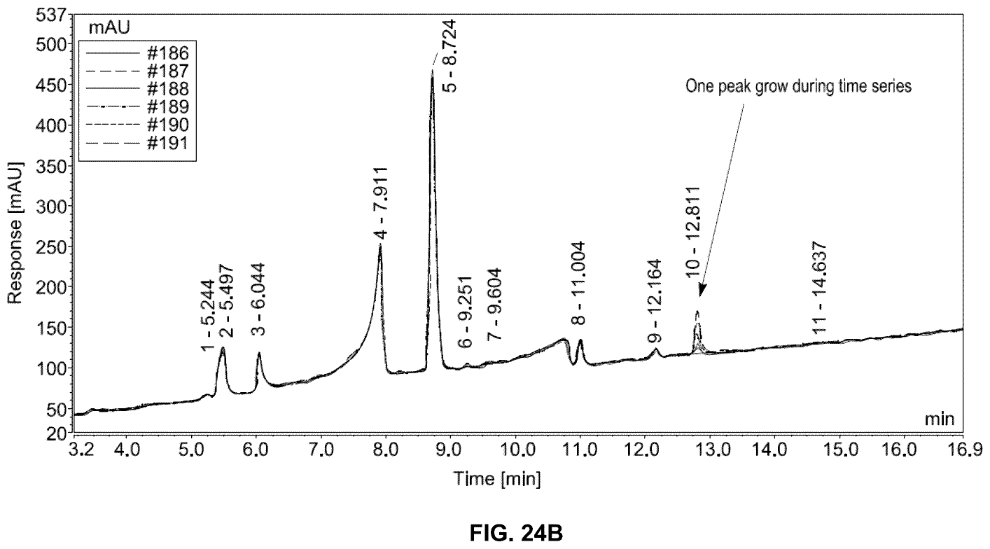

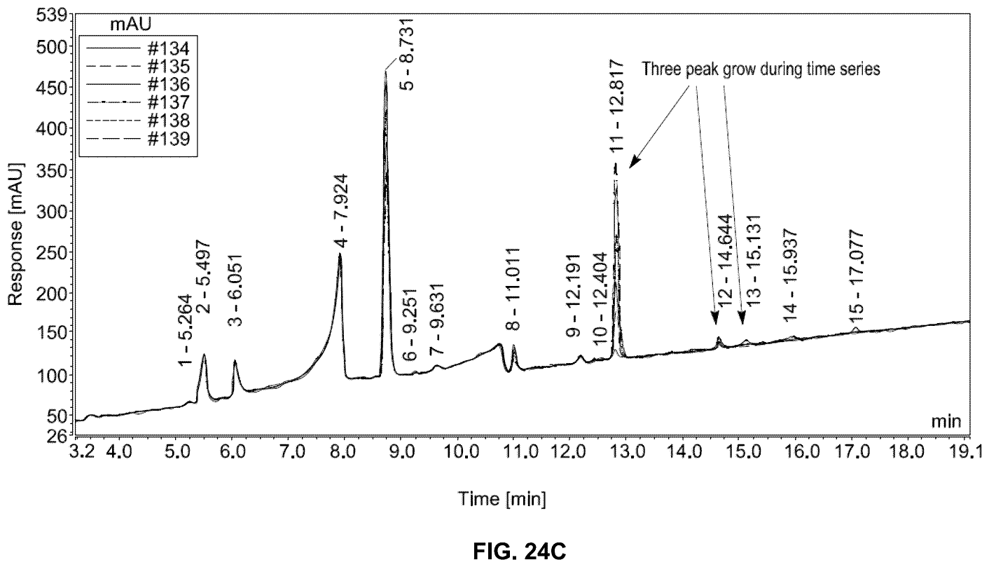

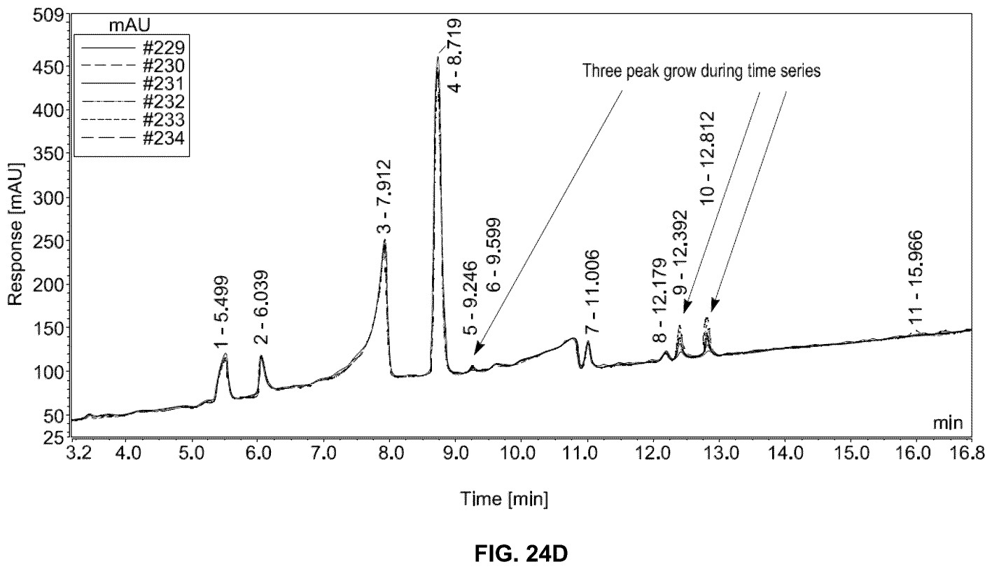

Results from the UHPLC analyses showed that all four GTF enzymes were capable of glucosylating rebaudioside A and stevioside, which was evident from the decrease of the peak area at retention times of approximately 11 minutes (rebaudioside A, FIGS. 23A-D) and 8.7 minutes (stevioside, FIGS. 24A-D). All the GTF enzymes produced a reaction product with rebaudioside A having a peak area growth over time at the retention time of 14.6 minutes (FIGS. 23A-D). GTFJ produced an additional reaction product from rebaudioside A glucosylation having a peak area growth over time at the retention time of 17.4 minutes (FIG. 23A). None of the glucosylated rebaudioside A products as analysed by UHPLC correlated with known standards for rubusoside, dulcoside A, rebaudioside B, rebaudioside C, rebaudioside F, rebaudioside A, rebaudioside E, rebaudioside M, rebaudioside D, rebaudioside N, or stevioside (data not shown).

With regard to reactions including stevioside, all the GTF enzymes mainly produced a glucosylated stevioside product having a peak area growth over time at the retention time of 12.8 minutes (FIGS. 24A-D). GTFJ and GTF 0768 furthermore produced a glucosylated stevioside product having a peak area growth over time at the retention time of 9.2 minutes and 12.4 minutes (FIGS. 24A and 24D). GTFJ and GTF 0974 had additional glucosylated stevioside products at retention times of 14.6 minutes and 15.1 minutes or 15.9 minutes (FIGS. 24A and 24C). None of the glucosylated stevioside products as analysed by UHPLC correlated with known standards for rubusoside, dulcoside A, rebaudioside B, rebaudioside C, rebaudioside F, rebaudioside A, rebaudioside E, rebaudioside M, rebaudioside D, rebaudioside N or stevioside (data not shown).

In contrast, no clear glucosylated products were observed when using the tested GTFs in reactions having rebaudioside M or rebaudioside D (data not shown).

### Example 11

### Glucosylation of Steviol Glycosides Rebaudioside a and Stevioside in Yogurt Using Glucosyltransferase Enzymes In Situ

The ability of GTF 0768 (SEQ ID NO:1) and vGTFJ (SEQ ID NO:3) to glucosylate rebaudioside A and stevioside were further investigated in a yogurt production set-up (scale: 60 g). Fresh milk was standardized to 4.0% (w/w) protein, 1.0% (w/w) fat, 8.0% (w/w) sucrose, which preparation was homogenized and pasteurized as described above (Materials/Methods), after which GTF 0768 or vGTFJ was added to four samples each (resulting in a total of eight samples) (the amount of each enzyme was that which corresponded to the enzyme volume needed to fully convert the sucrose under the fermentation conditions). Cultures were then added to each sample for fermentation to yogurt (Materials/Methods). Rebaudioside A (0.006 wt %) and stevioside (0.005 wt %) were individually added (final concentrations listed) to samples either at the beginning of yogurt fermentation or after fermentation was complete (Table 10).

TABLE 10

| | | Steviol glycoside | Steviol glycoside |
|---|---|---|---|
| Sample | GTF | included in | added after |
| No. | enzyme | fermentation | fermentation |
| 1 | vGTFJ | rebaudioside A | — |
| 2 | vGTFJ | — | rebaudioside A |
| 3 | GTF 0768 | rebaudioside A | — |
| 4 | GTF 0768 | — | rebaudioside A |
| 5 | vGTFJ | stevioside | — |
| 6 | vGTFJ | — | stevioside |
| 7 | GTF 0768 | stevioside | — |
| 8 | GTF 0768 | — | stevioside |

Following fermentation, the yoghurt samples were analysed by UHPLC (Materials/Methods). A glucosylated reaction product was identified in samples 1 and 3, which had included rebaudioside A during yogurt fermentation (Table 10). Both these samples in UHPLC analysis presented with a peak at the retention time of 14.6 minutes. Furthermore, a glucosylated reaction product was identified in sample 5, which had included stevioside during yogurt fermentation (Table 10). Sample 5 in UHPLC analysis presented with a peak at the retention time of 12.8 minutes. These results indicate that including the steviol glycosides stevioside or rebaudioside A during fermentation allow them to be accessible as acceptor molecules for GTF enzymes, whereas adding these steviol glycosides after fermentation renders them unavailable as GTF acceptor molecules.

What is claimed is:

1. A method of glucosylating a steviol glycoside, the method comprising:
providing a composition that comprises at least water, sucrose, a steviol glycoside, and a glucosyltransferase enzyme,
wherein the steviol glycoside comprises stevioside or rebaudioside A, and
wherein the glucosyltransferase enzyme is selected from:
(i) a glucosyltransferase enzyme that synthesizes alpha-1,6-glucan,
wherein at least about 50% of the glycosidic linkages of the alpha-1,6-glucan are alpha-1,6 linkages, and/or
(ii) a glucosyltransferase enzyme that synthesizes alpha-1,3-glucan,
wherein at least about 50% of the glycosidic linkages of the alpha-1,3-glucan are alpha-1,3 linkages,
wherein at least one glucosylated form of the steviol glycoside is produced in the composition, and typically wherein at least one alpha-glucan is produced in the composition.

2. The method of claim 1, wherein:
the glucosyltransferase enzyme that synthesizes alpha-1,6-glucan comprises an amino acid sequence that is at least 90% identical to SEQ ID NO:1, 2, 11, or 12, and/or
the glucosyltransferase enzyme that synthesizes alpha-1,3-glucan comprises an amino acid sequence that is at least 90% identical to residues 55-960 of SEQ ID NO:5, residues 54-957 of SEQ ID NO:6, residues 55-960 of SEQ ID NO:7, residues 55-960 of SEQ ID NO:8, residues 55-960 of SEQ ID NO:9, or SEQ ID NO:13.

3. The method of claim 1, wherein the alpha-glucan is produced in the composition.

4. The method of claim 1, wherein the composition is a food product/precursor.

5. The method of claim 4, wherein the food product/precursor is a dairy food product/precursor, optionally wherein the food product/precursor is a yogurt product/precursor.

6. The method of claim 4, further comprising: fermenting the food product/precursor after the providing step.

7. The method of claim 1, wherein the composition comprises the steviol glycoside by virtue of comprising a Stevia sweetener, Stevia extract, and/or other preparation from a Stevia rebaudiana plant, typically whereby the composition further comprises one or more steviol glycosides selected from dulcoside A, rebaudioside B, rebaudioside C, rebaudioside D, rebaudioside E, rebaudioside F, rebaudioside M, rubusoside, and/or steviolbioside.

8. A composition, or a glucosylated steviol glycoside, produced by the method of claim 1.

9. The composition of claim 8, wherein the composition is a food product/precursor.

10. A composition comprising a glucosylated steviol glycoside,
wherein the glucosylated steviol glycoside is produced by contacting a steviol glycoside with a glucosyltransferase enzyme in the presence of at least water and sucrose,
wherein the steviol glycoside comprises stevioside or rebaudioside A, and
wherein the glucosyltransferase enzyme is selected from:
(i) a glucosyltransferase enzyme that synthesizes alpha-1,6-glucan,
wherein at least about 50% of the glycosidic linkages of the alpha-1,6-glucan are alpha-1,6 linkages, and/or
(ii) a glucosyltransferase enzyme that synthesizes alpha-1,3-glucan,
wherein at least about 50% of the glycosidic linkages of the alpha-1,3-glucan are alpha-1,3 linkages.

11. The composition of claim 10, wherein the composition is a food product/precursor.
